(19) United States
(12) Patent Application Publication
(10) Pub. No.: US 2017/0037117 A1
(43) Pub. Date: Feb. 9, 2017

(54) COMPOSITIONS AND METHODS FOR TREATING METABOLIC DISORDERS

(71) Applicants: Salk Institute for Biological Studies, La Jolla, CA (US); The Regents of the University of California, Oakland, CA (US); Labrys Biologics, Inc., Redwood City, CA (US)

(72) Inventors: Andrew Dillin, Oakland, CA (US); Celine Riera, Berkeley, CA (US); Corey Goodman, Marshall, CA (US)

(73) Assignees: Salk Institute for Biological Studies, La Jolla, CA (US); The Regents of the University of California, Oakland, CA (US); Labrys Biologics, Inc., Redwood City, CA (US)

(21) Appl. No.: 15/290,869

(22) Filed: Oct. 11, 2016

Related U.S. Application Data
(63) Continuation of application No. PCT/US2015/031216, filed on May 15, 2015.
(60) Provisional application No. 61/994,739, filed on May 16, 2014.

(51) Int. Cl. C07K 16/18

(52) U.S. Cl. CPC C07K 16/18; C07K 2317/92; C07K 2317/34; C07K 2317/565; C07K 2317/76; C07K 2317/51; C07K 2317/33; C07K 2317/55; A61K 2039/505

(57) ABSTRACT

The disclosure features compositions and methods for treating a metabolic disorder, including diabetes, conditions associated with inhibition of insulin secretion, or for increasing longevity. In some embodiments, the methods comprise administering an anti-CGRP antagonist antibody.

### CROSS-REFERENCE TO RELATED APPLICATION

This application is a continuation of International Application No. PCT/US2015/031216 filed on May 15, 2015, which was published in English under PCT Article 21(2), which in turn claims the benefit of U.S. Provisional Application No. 61/994,739 filed on May 16, 2014, both herein incorporated by reference.

### SEQUENCE LISTING

The instant application contains a Sequence Listing which has been submitted electronically in ASCII format and is hereby incorporated by reference in its entirety. Said ASCII copy, created on Oct. 7, 2016, is named Sequence-Listing.txt and is 60,252 bytes in size.

### BACKGROUND

CGRP (calcitonin gene-related peptide) is a 37 amino acid neuropeptide, which belongs to a family of peptides that includes calcitonin, adrenomedullin and amylin. In humans, two forms of CGRP (α-CGRP and β-CGRP) exist and have similar activities. They vary by three amino acids and exhibit differential distribution. At least two CGRP receptor subtypes may also account for differential activities. CGRP is a neurotransmitter in the central nervous system, and has been shown to be a potent vasodilator in the periphery, where CGRP-containing neurons are closely associated with blood vessels. CGRP-mediated vasodilatation is also associated with neurogenic inflammation, as part of a cascade of events that results in extravasation of plasma and vasodilation of the microvasculature and is present in migraine.

Metabolic disorders (e.g., obesity, diabetes, etc.) and conditions associated with aging are two overlapping and mounting public health problems. Metabolic disorders have a number of indicia reflecting abnormalities in lipid and glucose metabolism. These abnormalities are considered to be a reason for atherosclerosis, cardiovascular diseases (CVD), diabetes mellitus, and the like. The frequency of metabolic disorders depends on the lifestyle and age. Metabolic disorder in an elderly population are a proven risk factor for mortality or cardiovascular (CV) morbidity, especially stroke and coronary heart disease (CHD). The high prevalence of metabolic disorders, heart attacks and diabetes in the elderly population makes the evidence of age an independent risk factor of the development of metabolic abnormalities. Therefore, there exists a pressing need for a treatment for metabolic disorders or to promote longevity.

### SUMMARY

There exists a pressing need for methods for treating a metabolic disorder in a subject. The present disclosure addresses these needs.

The disclosure provides methods for treating a metabolic disorder. The methods can include administering to a subject suffering a metabolic disorder a therapeutically effective amount of an anti-CGRP antagonist antibody. Exemplary metabolic disorders include, but are not limited to, weight gain, diabetes, cardiovascular diseases, or combinations thereof. In some embodiments, the metabolic disorder is diabetes. In some embodiments, the metabolic disorder is a disorder characterized by inhibition of insulin secretion.

In some embodiments, the subject can show increased insulin secretion, increased glucose tolerance and increased longevity after administering therapeutically effective amounts of one or more anti-CGRP antagonist antibodies.

In practicing any of the methods provided herein, the antibody can be a human or humanized antibody. In some embodiments, the antibody is a monoclonal antibody.

In some embodiments, the subject disclosed herein is a mammal, such as a human.

The methods disclosed herein can involve administering a therapeutically effective amount of an anti-CGRP antagonist antibody that binds CGRP. In some cases, the anti-CGRP antagonist antibody binds CGRP with a KD of 50 nM or less as measured by surface plasmon resonance at 37° C. In some cases, the anti-CGRP antagonist antibody has a half-life in vivo of at least 7 days.

In some embodiments, the anti-CGRP antagonist antibody can specifically bind to the C-terminal region of CGRP. In some cases, the anti-CGRP antagonist antibody specifically recognizes the epitope defined by the sequence GSKAF (SEQ ID NO: 48). In some cases, the anti-CGRP antagonist antibody includes a VH domain having the amino acid sequence shown in SEQ ID NO: 1 or 155. In some cases, the anti-CGRP antagonist antibody includes a VL domain having the amino acid sequence shown in SEQ ID NO: 2 or 156.

In some embodiments, the anti-CGRP antagonist antibody includes: (a) CDR H1 as set forth in SEQ ID NO: 3, 169, or 170; CDR H2 as set forth in SEQ ID NO: 4 or 171; CDR H3 as set forth in SEQ ID NO: 5; CDR L1 as set forth in SEQ ID NO: 6; CDR L2 as set forth in SEQ ID NO: 7; and CDR L3 as set forth in SEQ ID NO: 8; (b) CDR H1 as set forth in SEQ ID NO: 157, 172, or 173; CDR H2 as set forth in SEQ ID NO: 158 or 174; CDR H3 as set forth in SEQ ID NO: 159; CDR L1 as set forth in SEQ ID NO: 160; CDR L2 as set forth in SEQ ID NO: 161; and CDR L3 as set forth in SEQ ID NO: 162; or (c) a variant of an antibody according to (a) as shown in Table 6. In some cases, the anti-CGRP antagonist antibody can include a VH domain having the amino acid sequence shown in SEQ ID NO: 1 and a VL domain having the amino acid sequence shown in SEQ ID NO: 2.

In some embodiments, the anti-CGRP antagonist antibody is produced by the expression vectors with American Type Culture Collection (ATCC) Accession Nos. PTA-6867 and/or PTA-6866.

In some embodiments, the anti-CGRP antagonist antibody includes: the antibody G1 heavy chain full antibody amino acid sequence shown in SEQ ID NO: 11, with or without the C-terminal lysine; and the antibody G1 light chain full antibody amino acid sequence shown in SEQ ID NO: 12. In some cases, the anti-CGRP antagonist antibody includes: the antibody G2 heavy chain full antibody amino acid sequence shown in SEQ ID NO: 165; and the antibody G2 light chain full antibody amino acid sequence shown in SEQ ID NO: 166.

The anti-CGRP antagonist antibody can be administered using any routine method, such as peripherally. In some examples, the anti-CGRP antagonist antibody is administered orally, sublingually, via inhalation, transdermally, subcutaneously, intravenously, intra-arterially, intra-articularly, peri-articularly, locally and/or intramuscularly. In some examples, the anti-CGRP antagonist antibody is administered subcutaneously or intravenously.

In some embodiments, the anti-CGRP antagonist antibody can act peripherally upon administration.

In some embodiments, anti-CGRP antagonist antibody: (a) binds to CGRP; (b) blocks CGRP from binding to its receptor; (c) blocks or decreases CGRP receptor activation; (d) inhibits blocks, suppresses or reduces CGRP biological activity; (e) increases clearance of CGRP; and/or (g) inhibits CGRP synthesis, production or release.

The foregoing and other objects and features of the disclosure will become more apparent from the following detailed description, which proceeds with reference to the accompanying figures.

### BRIEF DESCRIPTION OF THE DRAWINGS

FIG. 1 is a table showing binding affinities of 12 murine antibodies for different alanine substituted human α-CGRP fragments. Binding affinities were measured at 25° C. using Biacore by flowing Fabs across CGRPs on the chip. The boxed values represent the loss in affinity of alanine mutants relative to parental fragment, 25-37 (italic), except K35A, which was derived from a 19-37 parent. “a” indicates affinities for 19-37 and 25-37 fragments are the mean average±standard deviation of two independent measurements on different sensor chips. “^{b}” indicates these interactions deviated from a simple bimolecular interaction model due to a biphasic offrate, so their affinities were determined using a conformational change model. Grey-scale key: white (1.0) indicates parental affinity; light grey (less than 0.5) indicates higher affinity than parent; dark grey (more than 2) indicates lower affinity than parent; and black indicates that no binding was detected.

FIGS. 2A and 2B show the effect of administering CGRP 8-37 (400 nmol/kg), antibody 4901 (25 mg/kg), and antibody 7D11 (25 mg/kg) on skin blood flow measured as blood cell flux after electrical pulse stimulation for 30 seconds. CGRP 8-37 was administered intravenously (iv) 3-5 min before electrical pulse stimulation. Antibodies were administered intraperitoneal (IP) 72 hours before electrical pulse stimulation. Each point in the graphs represents AUC of one rat treated under the conditions as indicated. Each line in the graphs represents average AUC of rats treated under the condition as indicated. AUC (area under the curve) equals to Δflux×Δtime. “Δflux” represents the change of flux units after the electrical pulse stimulation; and “Δtime” represents the time period taken for the blood cell flux level to return to the level before the electrical pulse stimulation.

FIG. 3 shows the effect of administering different dosage of antibody 4901 (25 mg/kg, 5 mg/kg, 2.5 mg/kg, or 1 mg/kg) on skin blood flow measured as blood cell flux after electrical pulse stimulation for 30 seconds. Antibodies were administered intravenously (IV) 24 hours before electrical pulse stimulation. Each point in the graph represents AUC of one rat treated under the conditions as indicated. The line in the graph represents average AUC of rats treated under the condition as indicated.

FIGS. 4A and 4B show the effect of administering antibody 4901 (1 mg/kg or 10 mg/kg, i.v.), antibody 7E9 (10 mg/kg, i.v.), and antibody 8B6 (10 mg/kg, i.v.) on skin blood flow measured as blood cell flux after electrical pulse stimulation for 30 seconds. Antibodies were administered intravenously (i.v.) followed by electrical pulse stimulation at 30 min, 60 min, 90 min, and 120 min after antibody administration. Y axis represents percent of AUC as compared to level of AUC when no antibody was administered (time 0). X axis represents time (minutes) period between the administration of antibodies and electrical pulse stimulation. “*” indicates P<0.05, and “**” indicates P<0.01, as compared to time 0. Data were analyzed using one-way ANOVA with a Dunnett's Multiple comparison test.

FIG. 5 shows the amino acid sequence of the heavy chain variable region (SEQ ID NO: 1) and light chain variable region (SEQ ID NO: 2) of antibody G1. The Kabat CDRs are in bold text, and the Chothia CDRs are underlined. The amino acid residues for the heavy chain and light chain variable region are numbered sequentially.

FIG. 6 shows epitope mapping of antibody G1 by peptide competition using Biacore. N-biotinylated human α-CGRP was captured on SA sensor chip. G1 Fab (50 nM) in the absence of a competing peptide or pre-incubated for 1 h with 10 uM of a competing peptide was flowed onto the chip. Binding of G1 Fab to the human α-CGRP on the chip was measured. Y axis represents percentage of binding blocked by the presence of the competing peptide compared with the binding in the absence of the competing peptide.

FIGS. 7A-7D show that TRPV1 disruption can extend mouse lifespan. FIGS. 7A-7B: Kaplan-Meyer survival curves for wild-type (WT) and TRPV1 knockout (KO) mice are shown. FIG. 7A: male data; FIG. 7B female data. Survival data are reported in Table 9. FIG. 7C: Necropsy analysis. Incidence of neoplastic and non-neoplastic diseases. Results reported as percentage of mice showing occurrence of either neoplastic or strictly non-neoplastic diseases, (p<0.1, ns, Fisher's exact test); FIG. 7D: Nature of neoplastic diseases. Results reported as percentage (total number) of mice with the disease, *p<0.05, Fisher's exact test. Ns: not significant, na: not applicable.

FIGS. 8A-E are diagrams and illustration demonstrating healthspan assessment in TRPV1 mutant mice. FIG. 8A: Barnes Maze procedure. 30 months male mice (n=10) were trained during four days, three trials per day, to find an escape hole on a brightly illuminated maze containing 20 equally spaced holes, with only one being the escape hole. After a two day interval, mice were retested on a retention test. Finally, a reversal memory test challenged the mice to relocate the escape hole placed 90° from the original hole. FIG. 8B: Barnes maze escape latency over the training, retention and reversal phases (two-way Anova, **p<0.005, * p<0.01). In the reversal phase, the primary path length to the hole was also measured. Even though TRPV1 KO mice tended to locate the hole faster than WT, they displayed decreased motivation to hide (i.e., enter the hole), which might be explained by reduced anxiety in the TRPV1 mutant mice. Therefore we measured the primary path length to the hole and found that TRPV1 mutant mice moved more directly to the escape box than their WT counter mates. FIG. 8C: Barnes maze errors (two-way Anova, ***p<0.0001, * p<0.01). FIG. 8D: Latency to fall from rotating rod at 4, 12 and 24 months (Student's t test, *p<0.01) FIG. 8E: Upper panel. Distribution of Nissl-positive neurons in the CA1, CA3, and DG areas of the hippocampus of TRPV1 KO and WT males at 24 months. Lower panel. Representative Hematoxylin and Eosin stainings of brain sections from TRPV1 KO and WT males at 24 months (Bregma—1.64 mm).

FIGS. 9A-9E are diagrams and images illustrating the evaluation of the requirement of the GH/IGF-1 axis in TRPV1 mutant mice. FIG. 9A: Representative pictures of adult TRPV1 KO (6 months old, left: WT, right: TRPV1 KO). FIG. 9B: Whole body growth of TRPV1 KO and WT mice from 3 to 24 months old, (left) male data (TRPV1 KO, n=38; WT, n=32), (right) female data (TRPV1 KO, n=48; WT, n=42). Means±S.E.M., n.s., two-way ANOVA. FIG. 9C: Body composition of 24 months old TRPV1 KO and WT males. Organ weight of lungs, liver, kidney, spleen, pancreas, brain, heart, iWAT (inguinal white adipose tissue), gWAT (gonadal white adipose tissue), pWAT (peritoneal white adipose tissue) are expressed as a percentage of total body weight. Means±S.E.M., n.s., Student's t test. FIG. 9D: Western blotting of growth hormone (GH) from pituitary glands (n=5, 6 months old). Loading control: β-actin. (E) ELISA analysis of plasma IGF-1 levels in fed male mice (n=15, 6 months old). Means±S.E.M., n.s., Student's t test. FIG. 9E is a bar graph showing plasma levels of IGF-1.

FIG. 10 shows the histological profiling of the TRPV1 mutant mice. Representative Hematoxylin and Eosin staining of tissue sections of TRPV1 KO and WT males at 24 months.

FIGS. 11A-11F illustrate metabolic profiling of the TRPV1 mutant mice. FIG. 11A: Mean rectum body temperature and food intake measurement of 6 months old males TRPV1 KO and WT (n.s., Student's t test). FIG. 11B: Fasting insulin, leptin, adiponectin, cholesterol and triglycerides of 6 months old males TRPV1 KO and WT (n.s., Student's t test). FIG. 11C: qPCR analysis of thermogenesis in BAT at 24 months. (n=5, means±SEM, n.s., Student's t test). FIG. 11D: Oxygen consumption (FIG. 11D) VO2 and (FIG. 11E) Total ambulatory activity at 3 months and 16 months. FIG. 11F: % KI67+/insulin+% and % CK20+/insulin+% in TRPV1 KO group and WT group. **p<0.001, Student's t test (n=8, means±SEM).

FIGS. 12A-12H illustrate the analysis of whole body energy expenditure and insulin resistance. FIG. 12A: Respiratory exchange ratio (RER) analysis of males TRPV1 KO and WT controls. n=8, means±S.E.M., ***p<0.0004, one-way ANOVA. FIG. 12B: Glucose tolerance tests at 3 months and 22 months, n=16, means±S.E.M., *** p<0.0001, **p<0.001, *p<0.01, Student's t test. FIG. 12C: Insulin tolerance test at 3 months and 22 months expressed as a percentage of the initial glucose levels, n=11-16, means±S.E.M., **p<0.001, *p<0.01, Student's t test. FIG. 12D: Pancreata were fixed and immuno-labeled against insulin (red) and glucagon (green), dapi nuclear dye (blue). FIG. 12E: β-cell mass of 24 months old TRPV1 KO and WT mice, n=4, means±S.E.M., *p<0.05, Student's t test. FIG. 12F: Quantitative PCR analysis of isolated pancreatic β-cells from TRPV1 KO and WT mice, n=8, means±S.E.M., *p<0.01, Student's t test. FIG. 12G: Glucose stimulated insulin secretion assay, n=5, means±S.E.M., *p<0.01, Student's t test. FIG. 12H: Western blot analysis of phospho-AKT (Ser473) and total AKT in TRPV1 KO and WT muscle (left) and liver (right) (n=5). α-tubulin and β-actin were respectively used as loading controls for muscle and liver tissues.

FIGS. 13A-13D are diagrams and images illustrating that trpv mutations in C. elegans require crtc1 to regulate lifespan. FIG. 13A: Survival curves of trpv double null mutant worms osm-9 (ky10); ocr-2 (ak47) compared to single mutants. FIG. 13B: Representatives images of CRTC1::RFP cytoplasmic to nuclear shuttling in intestinal cells upon tricaine treatment in trpv mutant (osm-9 (ky10); ocr-2 (ak47)) worms and WT control. FIG. 13C: Survival curves of transgenic worms expressing CRTC1::RFP on calcineurin (tax-6) RNAi (dashed lines) compared to control worms fed GFP RNAi (plain lines). FIG. 13D: Survival curves of the TRPV mutants in a constitutively nuclear CRTC1 strain mutated at the tax-6 dephosphorylation sites (576A, S179A) compared to WT controls.

FIGS. 14A-14E show that nuclear translocation of CRTC1 requires activation of TRPV1. FIG. 14A: Schematic representing the modulation of cAMP signaling and TRPV1 activity on CRTC1 shuttling in DRG neurons upon the pharmacological manipulations used in FIGS. 14B-D. FIGS. 14B-14D: Maximal projections of z-stack sections throughout the nucleus of the neuronal bodies. FIG. 14B: Wild-type mouse DRGs primary cultures were immunostained with antibodies against TRPV1 (red), CRTC1 (green), dapi (blue). FIG. 14C: left. DRG neuronal cultures were subjected to FSK (25 μM), agonist of L-type calcium channels inducing cAMP release, and Cap (1 μM), a potent TRPV1 agonist. Immunostaining was performed after fixation with antibodies against CGRP (red), CRTC1 (green), dapi (blue). right. Quantification of the mean nuclear to cytoplasmic ratio, relative to vehicle treatment FIG. 14D: left. WT DRGs cultures were pretreated with vehicle, the calcineurin inhibitor CsA (10 μM) and the TRPV1 antagonist SB-366791 (10 μM) prior to 1 hour stimulation with FSK and Cap. Neurons were fixed and immunostained with antibodies against TRPV1 (red), CRTC1 (green), dapi (blue). right. Quantification of the mean nuclear to cytoplasmic ratio, relative to vehicle treatment. (FIGS. 14C-14D) n=90-120 cells analyzed, means±S.E.M., ***p<0.0001, Student's t test. FIG. 14E: Quantitative PCR analysis was carried out to examine CREBdependent transcript induction in untreated DRG neurons from TRPV1 KO and WT. N=7, means±S.E.M. (*p<0.05, Student's t test).

FIGS. 15A-15F show that CGRP is a neuroendocrine regulator of the metabolism. FIGS. 15A-15C: Insulin secretion assay of mouse insulinoma cells (MIN6) upon 16.8 mM glucose stimulation. Cells were incubated with 0.1-0.5 μM synthetic peptides for FIG. 15A: rat CGRPα, FIG. 15B: rat CGRPβ and FIG. 15C: rat Substance P, n=4, means±S.E.M, **p<0.01, *p<0.05, Student's t test. FIG. 15D: Serum levels of CGRP measured by ELISA in blood samples from WT and TRPV1 mutants at 3 and 24 months, n=7, means±S.E.M. a, ns and b, p<0.05. FIG. 15E: Respiratory exchange ratio (RER) analysis and oxygen consumption FIG. 15F: of 23 months old male WT animals treated with CGRP8-37 or vehicle control, n=10, means±S.E.M.,**p<0.01, *p<0.05, Student's t test.

FIGS. 16A-16C illustrate insulin secretion and inflammation of the TRPV1 mutant mice. FIG. 16A: Insulin secretion assay of mouse insulinoma cells (MIN6) upon 16.8 mM glucose stimulation with 0.1-0.5 μM synthetic human CGRPα. FIG. 16B: Insulin secretion assay (MIN6) upon 16.8 mM glucose and exendin 4 stimulation. Cells were incubated with 0.1-0.5 μM synthetic peptides for h CGRPα, rat CGRPβ and rat Substance P, n=4, means±S.E.M, **p<0.01, *p<0.05, Student's t test. FIG. 16C: qPCR analysis of chemokines and pro-inflammatory genes at 24 months in brain, quadriceps muscle, gonadal fat and pancreas at 24 months (n=5, means±SEM, n.s., Student's t test).

FIG. 17 is a schematic diagram illustrating a model for the neuroendocrine regulation of metabolism by TRPV1 expressing neurons. Stimulation of TRPV1 by external stimuli promotes CGRP secretion from DRG neurons onto the pancreatic β-cells and inhibition of insulin release. TRPV1 activation results in calcium influx, activation of calcineurin allowing dephosphorylation of CRTC1 and release from 14-3-3 proteins, resulting in nuclear internalization of CRTC1 and transcription of its targets, such as CGRP. CGRP accumulation has detrimental effects on energy expenditure, glucose tolerance and aging. In contrast, loss of TRPV1 promotes lifespan extension through increased insulin secretion, metabolic health by inactivating of the CRTC1/CREB signaling cascade.

FIG. 18 is an alignment of human and rat CGRP amino acid sequences.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NO: 1 is a G1 heavy chain variable region amino acid sequence.

SEQ ID NO: 2 is a G1 light chain variable region amino acid sequence.

SEQ ID NO: 3 is a G1 CDR H1 (extended CDR) amino acid sequence.

SEQ ID NO: 4 is a G1 CDR H2 (extended CDR) amino acid sequence.

SEQ ID NO: 5 is a G1 CDR H3 amino acid sequence.

SEQ ID NO: 6 is a G1 CDR L1 amino acid sequence.

SEQ ID NO: 7 is a G1 CDR L2 amino acid sequence.

SEQ ID NO: 8 is a G1 CDR L3 (amino acid sequence.

SEQ ID NO: 9 is a G1 heavy chain variable region nucleotide sequence.

SEQ ID NO: 10 is a G1 light chain variable region nucleotide sequence.

SEQ ID NO: 11 is a G1 heavy chain full antibody amino acid sequence (including modified IgG2 as described herein).

SEQ ID NO: 12 is a G1 light chain full antibody amino acid sequence.)

SEQ ID NO: 13 is a G1 heavy chain full antibody nucleotide sequence (including modified IgG2 as described herein).

SEQ ID NO: 14 is a G1 light chain full antibody nucleotide sequence.

SEQ ID NO: 15 is a human α-CGRP amino acid sequence.

SEQ ID NO: 16 is a fragment of a human α-CGRP amino acid sequence (amino acids 8-37).

SEQ ID NO: 17 is a fragment of a human α-CGRP amino acid sequence (amino acids 19-37).

SEQ ID NO: 18 is a mutant fragment of a human α-CGRP amino acid sequence (P28A amino acids 19-37).

SEQ ID NO: 19 is a mutant fragment of a human α-CGRP amino acid sequence (K35K amino acids 19-37).

SEQ ID NO: 20 is a mutant fragment of a human α-CGRP amino acid sequence (K35E amino acids 19-37).

SEQ ID NO: 21 is a mutant fragment of a human α-CGRP amino acid sequence (K35M amino acids 19-37).

SEQ ID NO: 22 is a mutant fragment of a human α-CGRP amino acid sequence (K35Q amino acids 19-37).

SEQ ID NO: 23 is a mutant fragment of a human α-CGRP amino acid sequence (F37A amino acids 19-37).

SEQ ID NO: 24 is a mutant fragment of a human α-CGRP amino acid sequence (38A amino acids 25-38).

SEQ ID NO: 25 is a fragment of a human α-CGRP amino acid sequence (25-37).

SEQ ID NO: 26 is a mutant fragment of a human α-CGRP amino acid sequence (F27A amino acids 25-37).

SEQ ID NO: 27 is a mutant fragment of a human α-CGRP amino acid sequence (V28A amino acids 25-37).

SEQ ID NO: 28 is a mutant fragment of a human α-CGRP amino acid sequence (P29A amino acids 25-37).

SEQ ID NO: 29 is a mutant fragment of a human α-CGRP amino acid sequence (T30A amino acids 25-37).

SEQ ID NO: 30 is a mutant fragment of a human α-CGRP amino acid sequence (N31A amino acids 25-37).

SEQ ID NO: 31 is a mutant fragment of a human α-CGRP amino acid sequence (V32A amino acids 25-37).

SEQ ID NO: 32 is a mutant fragment of a human α-CGRP amino acid sequence (G33A amino acids 25-37).

SEQ ID NO: 33 is a mutant fragment of a human α-CGRP amino acid sequence (534A amino acids 25-37).

SEQ ID NO: 34 is a mutant fragment of a human α-CGRP amino acid sequence (F37A amino acids 25-37).

SEQ ID NO: 35 is a fragment of a human α-CGRP amino acid sequence (amino acids 26-37).

SEQ ID NO: 36 is an amidated fragment of human α-CGRP amino acid sequence (amino acids 19-37).

SEQ ID NO: 37 is an amidated fragment of a human α-CGRP amino acid sequence (amino acids 19-36).

SEQ ID NO: 38 is an amidated fragment of a human α-CGRP amino acid sequence (amino acids 1-36).

SEQ ID NO: 39 is an amidated fragment of a human α-CGRP amino acid sequence (amino acids 1-19).

SEQ ID NO: 40 is an amidated fragment of a human α-CGRP amino acid sequence (amino acids 1-13).

SEQ ID NO: 41 is a rat α-CGRP amino acid sequence.

SEQ ID NO: 42 is a fragment of a rat α-CGRP amino acid sequence (amino acids 19-37)

SEQ ID NO: 43 is a human β-CGRP amino acid sequence.

SEQ ID NO: 44 is a rat β-CGRP amino acid sequence.

SEQ ID NO: 45 is a human calcitonin amino acid sequence fragment (amino acids 1-32).

SEQ ID NO: 46 is a human amylin amino acid sequence fragment (amino acids 1-37).

SEQ ID NO: 47 is a human adrenomedullin amino acid sequence fragment (amino acids 1-52).

SEQ ID NO: 48 is a CGRP epitope sequence.

SEQ ID NOS: 49 to 55 are primer sequences.

SEQ ID NO: 56 is a 6-His tag sequence.

SEQ ID NO: 57 is a linker sequence.

SEQ ID NOS: 58 to 154 are primer sequences.

SEQ ID NO: 155 is an antibody G2 heavy chain variable region amino acid sequence.

SEQ ID NO: 156 is an antibody G2 light chain variable region amino acid sequence.

SEQ ID NO: 157 is an antibody G2 CDR H1 (Kabat CDR) amino acid sequence.

SEQ ID NO: 158 is an antibody G2 CDR H2 (extended CDR) amino acid sequence.

SEQ ID NO: 159 is an antibody G2 CDR H3 amino acid sequence.

SEQ ID NO: 160 is an antibody G2 CDR L1 amino acid sequence.

SEQ ID NO: 161 is an antibody G2 CDR L2 amino acid sequence.

SEQ ID NO: 162 is an antibody G2 CDR L3 amino acid sequence.

SEQ ID NO: 163 is an antibody G2 heavy chain variable region nucleotide sequence.

SEQ ID NO: 164 is antibody G2 light chain variable region nucleotide sequence.

SEQ ID NO: 165 is an antibody G2 heavy chain full antibody amino acid sequence (not including Fc domain).

SEQ ID NO: 166 is an antibody G2 light chain full antibody amino acid sequence.

SEQ ID NO: 167 is an antibody G2 heavy chain full antibody nucleotide sequence (not including Fc domain).

SEQ ID NO: 168 is an antibody G2 light chain full antibody nucleotide sequence.

SEQ ID NO: 169 is an antibody G1 CDR H1 (Chothia CDR) amino acid sequence.

SEQ ID NO: 170 is an antibody G1 CDR H1 (Kabat CDR) amino acid sequence.

SEQ ID NO: 171 is an antibody G1 CDR H2 (Chothia CDR) amino acid sequence.

SEQ ID NO: 172 is an antibody G2 CDR H1 (extended CDR) amino acid sequence.

SEQ ID NO: 173 is an antibody G2 CDR H1 (Chothia CDR) amino acid sequence

SEQ ID NO: 174 is an antibody G2 CDR H2 (Chothia CDR) amino acid sequence.

### DETAILED DESCRIPTION

The present disclosure provides methods for treating and/or preventing a metabolic disorder in a subject by administering to the subject a therapeutically effective amount of an anti-CGRP antagonist antibody. Also as described herein are methods for increasing longevity of a subject by administering to the subject a therapeutically effective amount of an anti-CGRP antagonist antibody. The anti-CGRP antagonist antibody can be an antibody that specifically binds CGRP. The disclosure also provides anti-CGRP antagonist antibodies and polypeptides derived from G1 or its variants shown in Table 6 of WO2007/054809, which is hereby incorporated by reference in its entirety.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. Furthermore, to the extent that the terms “including”, “includes”, “having”, “has”, “with”, or variants thereof are used in either the detailed description and/or the claims, such terms are intended to be inclusive in a manner similar to the term “comprising”. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, GenBank® Accession Nos., and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold, or within 2-fold, of a value. Where particular values are described in the application and claims, unless otherwise stated the term “about” meaning within an acceptable error range for the particular value should be assumed.

“Treatment”, “treating”, “palliating” and “ameliorating”, as used herein, are used interchangeably. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disorder, or to a patient reporting one or more of the physiological symptoms of a disorder, even though a diagnosis of this disorder may not have been made.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv, dAb), single chain antibodies (ScFv), mutants thereof, chimeric antibodies, diabodies, fusion proteins comprising an antibody portion, and any other modified configuration of the immunoglobulin molecule that comprises an antigen recognition site. An antibody can include an antibody of any class, such as IgG, IgA, or IgM (or sub-class thereof). Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins can be called alpha, delta, epsilon, gamma, and mu, respectively. The subunit structures and three-dimensional configurations of different classes of immunoglobulins are well known. “Fv” refers to an antibody fragment that contains a complete antigen-recognition and -binding site. In a two-chain Fv species, this region consists of a dimer of one heavy and one light chain variable domain in tight, non-covalent association. In a single-chain Fv species, one heavy and one light chain variable domain can be covalently linked by a flexible peptide linker such that the light and heavy chains can associate in a dimeric structure analogous to that in a two-chain Fv species. In this configuration, the three CDRs of each variable domain can interact to define an antigen-binding specificity on the surface of the VH-VL dimer. However, a single variable domain (or half of an Fv comprising only 3 CDRs specific for an antigen) can have the ability to recognize and bind antigen, although generally at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge regions. A F(ab)2 fragment is a bivalent fragment comprising two Fab fragments linked by a disulfide bridge at the hinge region.

An antibody can have one or more binding sites (for combining with antigen). In the cases when there is more than one binding site, the binding sites may be identical to one another or may be different. For instance, a naturally-occurring immunoglobulin can have two identical binding sites, a single chain antibody or Fab fragment can have one binding site, while a “bispecific” or “bifunctional” antibody (diabody) can have two different binding sites, in terms of sequence and/or antigen/epitope recognition.

An “isolated antibody” refers to an antibody that (1) is not associated with naturally-associated components, including other naturally-associated antibodies, that accompany it in its native state, (2) is free of other proteins from the same species, (3) is expressed by a cell from a different species, and/or (4) does not occur in nature.

A “monoclonal antibody” refers to a homogeneous antibody population wherein the monoclonal antibody is comprised of amino acids (naturally occurring and non-naturally occurring) that are involved in the selective binding of an antigen. A population of monoclonal antibodies is highly specific, being directed against a single antigenic site. The term “monoclonal antibody” encompasses not only intact monoclonal antibodies and full-length monoclonal antibodies, but also fragments thereof (such as Fab, Fab′, F(ab′)2, Fv), single chain (ScFv), mutants thereof, fusion proteins comprising an antibody portion, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized antibodies” refer to forms of non-human (e.g., murine) antibodies that are specific chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. For example, the sequences derived from non-human immunoglobulin can be less than 10%, less than 9%, less than 8%, less than 7%, less than 6%, less than 5%, less than 4%, less than 3%, less than 2%, less than 1%, less than 0.5%, less than 0.1% or less than 0.01%; or even substantially free of sequences derived from non-human immunoglobulin. Generally, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may include residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. In some examples, a humanized antibody will include at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

A “human antibody” is an antibody having an amino acid sequence corresponding to that of an antibody produced in a human and/or has been made using any of the techniques for making human antibodies known in the art or disclosed herein. A human antibody can include antibodies including at least one human heavy chain polypeptide or at least one human light chain polypeptide. One such example is an antibody that includes murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016, all hereby incorporated by reference in their entirety. In one example, a human antibody is prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

A “single chain antibody (scFc)” refers to an antibody in which VL and VH regions are paired to form a monovalent molecule via a synthetic linker that enables them to be made as a single protein chain (e.g., see Bird et al., Science, 242: 423-426 (1988) and Huston et al., Proc. Natl. Acad. Sci. USA, 85:5879-5883 (1988)).

“Diabodies” refer to bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

“Chimeric antibodies” refer to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. In these chimeric antibodies, the variable region of both light and heavy chains can mimic the variable regions of antibodies derived from one species of mammals, while the constant portions can be homologous to the sequences in antibodies derived from another. One advantage to such chimeric forms is that, for example without limitation, the variable regions can conveniently be derived from presently known sources using readily available hybridomas or B cells from non-human host organisms in combination with constant regions derived from, for example, human cell preparations. While the variable region can have the advantage of ease of preparation, and the specificity is not affected by its source, the constant region being human, can be less likely to elicit an immune response from a human subject when the antibodies are injected than would the constant region from a non-human source. The definition is not limited to this particular example.

A “functional Fc region” can possess at least one effector function of a native sequence Fc region. Exemplary “effector functions” include but are not limited to, Clq binding; complement dependent cytotoxicity (CDC); Fc receptor binding; antibody-dependent cell-mediated cytotoxicity (ADCC); phagocytosis; down-regulation of cell surface receptors (e.g., B cell receptor; BCR), and the like. Such effector functions generally require the Fc region to be combined with a binding domain (e.g., an antibody variable domain) and can be assessed using various assays known in the art for evaluating such antibody effector functions.

A “native sequence Fc region” includes an amino acid sequence identical to the amino acid sequence of an Fc region found in nature. A “variant Fc region” includes an amino acid sequence which differs from that of a native sequence Fc region by virtue of at least one amino acid modification, yet retains at least one effector function of the native sequence Fc region. In one example, the variant Fc region has at least one amino acid substitution compared to a native sequence Fc region or to the Fc region of a parent polypeptide, e.g., from about one to about ten amino acid substitutions, or from about one to about five amino acid substitutions in a native sequence Fc region or in the Fc region of the parent polypeptide (such as 1, 2, 3, 4, 5, 6, 7, 8, 9, or 10 amino acid substitutions). A variant Fc region in some examples possesses at least about 80% sequence identity with a native sequence Fc region and/or with an Fc region of a parent polypeptide, such as at least about 90% sequence identity therewith, at least about 95%, at least 96%, at least 97%, at least 98%, or at least 99% sequence identity therewith.

“Antibody-dependent cell-mediated cytotoxicity (ADCC) refers to a cell-mediated reaction in which nonspecific cytotoxic cells that express Fc receptors (FcRs) (e.g., natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Fc receptor (FcR) describes a receptor that binds to the Fc region of an antibody. The FcR can be a native sequence human FcR. Moreover, an exemplary FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

“Complement dependent cytotoxicity (CDC)” refers to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods, 202:163 (1996), may be performed.

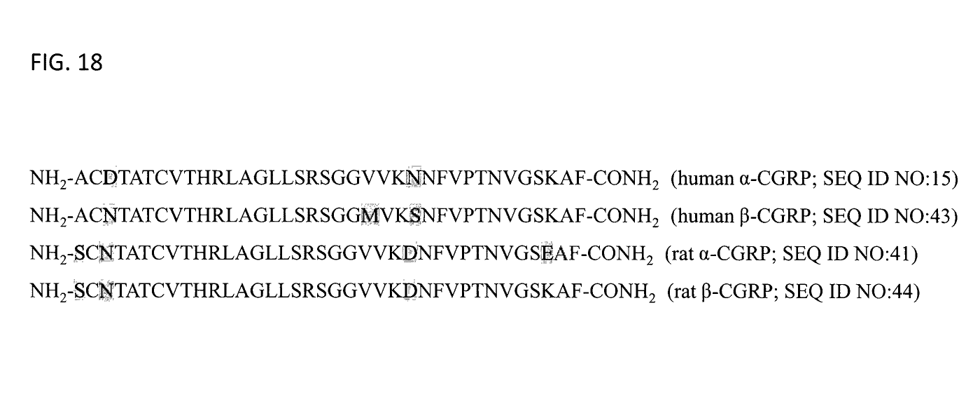

“Calcitonin gene-related peptide (CGRP)” refers to any form of calcitonin gene-related peptide and variants thereof that retain at least part of the activity of CGRP. For example, CGRP may be α-CGRP or β-CGRP. As used herein, CGRP includes all mammalian species of native sequence CGRP, e.g., human, canine, feline, equine, and bovine. Additional information on human α-CGRP can be found at OMIM 114130. Additional information on human β-CGRP can be found at OMIM 114160. CGRP sequences are publically available, for example from the GenBank® sequence database. One of ordinary skill in the art can identify additional CGRP nucleic acid and protein sequences. Other specific examples are provided herein (e.g., human α-CGRP (SEQ ID NO:15); human β-CGRP (SEQ ID NO:43); rat α-CGRP (SEQ ID NO:41); and rat β-CGRP (SEQ ID NO:44)). An alignment of exemplary CGRP sequences is shown in FIG. 18.

An “anti-CGRP antagonist antibody” refers to an antibody that is able to inhibit CGRP biological activity and/or downstream pathway(s) mediated by CGRP signaling. An anti-CGRP antagonist antibody encompasses antibodies that block, antagonize, suppress or reduce (including significantly) CGRP biological activity, including downstream pathways mediated by CGRP signaling, such as receptor binding and/or elicitation of a cellular response to CGRP. For purpose of the present disclosure, it will be understood that the term “anti-CGRP antagonist antibody” encompasses all the previously identified terms, titles, and functional states and characteristics whereby the CGRP itself, an CGRP biological activity (including but not limited to its ability to mediate any aspect of headache), or the consequences of the biological activity, are substantially nullified, decreased, or neutralized in any meaningful degree. In some embodiments, an anti-CGRP antagonist antibody binds CGRP and prevents CGRP binding to a CGRP receptor. In other embodiments, an anti-CGRP antagonist antibody binds CGRP and prevents activation of a CGRP receptor. Examples of anti-CGRP antagonist antibodies are provided herein. Antibodies that bind to CGRP are also referred to herein as “anti-CGRP antibodies.”

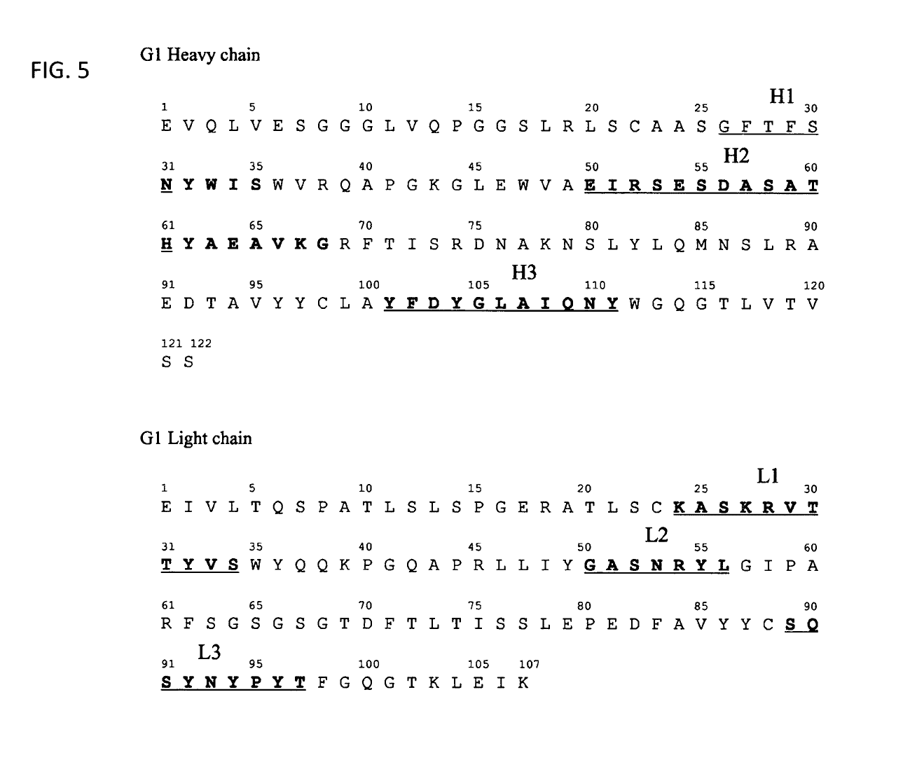

“G1” and “antibody G1” are used interchangeably to refer to an antibody produced by the expression vectors having deposit numbers ATCC-PTA-6867 and ATCC-PTA-6866. The amino acid sequence of the heavy chain and light chain variable regions are shown in SEQ ID Nos. 1 and 2. The CDR portions of antibody G1 (including Chothia and Kabat CDRs) are diagrammatically depicted in FIG. 5 of WO2007/054809, the content of which is herein incorporated by reference in its entirety. The polynucleotides encoding the heavy and light chain variable regions are shown in SEQ ID Nos. 9 and 10. The characterization of antibody G1 is described in the Examples of WO2007/054809, the entire content of which is herein incorporated by reference. G1 is a humanized monoclonal blocking antibody (IgG2) which blocks binding and activity of the neuropeptide CGRP (a and b) and its effect of neurogenic vasodilatation caused by CGRP release. G1 is an IgG2.DELTA.a monoclonal anti-CGRP antagonist antibody derived from the murine anti-CGRP antagonist antibody precursor, denoted muMAb7E9 as identified in a screen using spleen cells prepared from a mouse immunized with human and rat CGRP that were fused with murine plasmacytoma cells. G1 was created by grafting the muMAb 7E9 derived CDRs of light and heavy chain into the closest human germ line sequence followed by the introduction of at least 1 mutation into each CDR and 2 framework mutations in VH. Two mutations were introduced into the Fc domain of G1 to suppress human Fc-receptor activation. G1 and muMab7E9 have been shown to recognize the same epitope.

“G2” and “antibody G2” are used interchangeably to refer to an anti-rat CGRP mouse monoclonal antibody as described in Wong et al., Hybridoma 12:93-106 (1993). The amino acid sequence of the heavy chain and light chain variable regions are shown in SEQ ID Nos. 155 and 156. The polynucleotides encoding the heavy and light chain variable regions are shown in SEQ ID Nos. 163 and 164. The CDR portions of antibody G2 are provided in SEQ ID Nos. 157 to 162. G2 has been shown to recognize the same epitope as G1.

“Immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins).

An epitope that “specifically binds”, or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. It is also understood that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. It is understood that, because the polypeptides provided herein are based upon an antibody, the polypeptides can occur as single chains or associated chains.

“Polynucleotide,” or “nucleic acid molecule,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may include modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR.sub.2 (“amidate”), P(O)R, P(O)OR′, CO or CH.sub.2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (e.g., Kabat et al. Sequences of Proteins of Immunological Interest, (5th ed., 1991, National Institutes of Health, Bethesda Md.)); and (2) an approach based on crystallographic studies of antigen-antibody complexes (e.g., Chothia et al. (1989) Nature 342:877; Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

“Substantially pure” refers to material which is at least 50% pure (i.e., free from contaminants), such as at least 90% pure, at least 95% pure, at least 98% pure, or at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

The terms “effective amount,” “therapeutically effective amount,” or “therapeutic amount” which is further described herein, encompasses both this lesser effective amount and the usual effective amount, and indeed, any amount that is effective to elicit a particular condition, effect, and/or response. As such, a dose of any such subject of concurrent administration may be less than that which might be used were it administered alone. One or more effect (s) of any such subject (s) of administration may be additive or synergistic. Any such subject(s) of administration may be administered more than one time. The effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down-regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

A “sub-therapeutic amount” of an agent or therapy is an amount less than the effective amount for that agent or therapy, an amount below which would be considered therapeutic. However when such amount is combined with an effective or a sub-therapeutic amount of another agent or therapy, it can produce a result desired by the physician, due to, for example, synergy in the resulting efficacious effects, or reduction of side effects of one or more of the agents used in the combination. For example, FDA guidelines can suggest a specified level of dosing to treat a particular condition, and a sub-therapeutic amount would be any level that is below the FDA suggested dosing level.

A “synergistically effective therapeutic amount” of an agent or therapy is an amount which, when combined with an effective or sub-therapeutic amount of another agent or therapy, produces a greater effect than when either of the two agents are therapies are used alone. In some embodiments, a synergistically effective therapeutic amount of an agent or therapy produces a greater effect when used in combination than the additive effects of each of the two agents or therapies when used alone.

A “therapeutic effect,” as used herein, encompasses a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Thus, the antibodies disclosed herein (e.g., anti-CGRP antagonist antibodies) can be combined with one or more pharmaceutically acceptable salts, such as those suitable for administration to a subject.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the disclosure is contemplated. Supplementary active ingredients can also be incorporated into the compositions. As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000). Thus, the antibodies disclosed herein (e.g., anti-CGRP antagonist antibodies) can be present in one or more pharmaceutically acceptable carriers suitable for administration to a subject.

In one embodiment, “prepared for” herein means the medicament is in the form of a dosage unit or the like suitably packaged and/or marked for use in peripheral administration.

A “biological sample” encompasses a variety of sample types obtained from an individual and can be used in a diagnostic or monitoring assay. The term encompasses blood and other liquid samples of biological origin, solid tissue samples such as a biopsy specimen or tissue cultures or cells derived therefrom, and the progeny thereof. The term also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

An “individual” or “subject” is a vertebrate, such as a mammal, for example a human. Mammals include, but are not limited to, farm animals (such as cows, pigs, sheep), sport animals, pets (such as cats, dogs and horses), primates, rabbits, mice and rats. The methods described herein can be useful in both human therapeutics, pre-clinical, and veterinary applications. In some embodiments, the subject is a mammal, and in some embodiments, the subject is human.

The term “in vivo” refers to an event that takes place in a subject's body.

A “vector” is a construct, which is capable of delivering, and in some examples expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

An “expression control sequence” is a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “peripherally administered” refers to the route by which the a substance, medicament and/or anti-CGRP antagonist antibody is to be delivered, in particular it means not centrally, not spinally, not intrathecally, not delivered directly into the CNS. The term refers to administration routes other than those immediately forgoing and includes via a route which is oral, sublingual, buccal, topical, rectal, via inhalation, transdermal, subcutaneous, intravenous, intra-arterial, intramuscular, intracardiac, intraosseous, intradermal, intraperitoneal, transmucosal, vaginal, intravitreal, intra-articular, peri-articular, local or epicutaneous. Thus, the antibodies disclosed herein (e.g., anti-CGRP antagonist antibodies) can be administered to a subject using any of these methods.

The term “acts peripherally” refers to the site of action of a substance, compound, medicament and/or anti-CGRP antagonist antibody said site being within the peripheral nervous system as opposed to the central nervous system, said compound, medicament and/or anti-CGRP antagonist antibody said being limited by inability to cross the barrier to the CNS and brain when peripherally administered. The term “centrally penetrating” refers to the ability of a substance to cross the barrier to the brain or CNS.

The term “kₒₙ” is intended to refer to the rate constant for association of an antibody to an antigen.

The term “k_{off}” is intended to refer to the rate constant for dissociation of an antibody from the antibody/antigen complex.

The term “K_{D}” is intended to refer to the equilibrium dissociation constant of an antibody-antigen interaction.

“Agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present disclosure.

Generally, the term “concurrent administration”, “co-administration”, or “administration in conjunction with” in reference to two or more subjects of administration for administration to a subject body, such as components, agents, substances, materials, compositions, and/or the like, refers to administration performed using dose(s) and time interval(s) such that the subjects of administration are present together within the subject body, or at a site of action in the subject body, over a time interval in less than de minimus quantities. The time interval may be any suitable time interval, such as an appropriate interval of minutes, hours, days, or weeks, for example. The subjects of administration may be administered together, such as parts of a single composition, for example, or otherwise. The subjects of administration may be administered substantially simultaneously (such as within less than or equal to about 5 minutes, about 3 minutes, or about 1 minute, of one another, for example) or within a short time of one another (such as within less than or equal to about 1 hour, 30 minutes, or 10 minutes, or within more than about 5 minutes up to about 1 hour, of one another, for example). The subjects of administration so administered may be considered to have been administered at substantially the same time. One of ordinary skill in the art will be able to determine appropriate dose(s) and time interval(s) for administration of subjects of administration to a subject body so that same will be present at more than de minimus levels within the subject body and/or at effective concentrations within the subject body. When the subjects of administration are concurrently administered to a subject body, any such subject of administration may be in an effective amount that is less than an effective amount that might be used were it administered alone.

“Diabetes mellitus” refers to a group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 2 diabetes results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level ≧7.0 mmol/l (126 mg/dl);
b. Plasma glucose ≧11.1 mmol/l (200 mg/dL) two hours after a 75 g oral glucose load as in a glucose tolerance test;
c. Symptoms of hyperglycemia and casual plasma glucose ≧11.1 mmol/l (200 mg/dl);
d. Glycated hemoglobin (Hb A1C)≧6.5%

Metabolic disorder/disease: A disease or disorder that results from the disruption of the normal mammalian process of metabolism. Includes metabolic syndrome. Examples include but are not limited to: (1) glucose utilization disorders and the sequelae associated therewith, including diabetes mellitus (Type I and Type-2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic β-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thromboses, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinfiammatory processes and/or other peripheral neuropathies, including Alzheimer's disease, multiple sclerosis, Parkinson's disease, progressive multifocal leukoencephalopathy and Guillian-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Other examples are provided in WO 2014/085365 (herein incorporated by reference).

In specific examples, the metabolic disease includes one or more of (such as at least 2 or at least 3 of): diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension).

### Methods for Treating Metabolic Disorders

There exists a clinical need for compositions and methods that are useful for effectively treating, preventing or delaying metabolic disorders or aging. Disclosed herein are compositions and methods for treating, preventing, slowing the progression of, controlling or ameliorating a metabolic disorder and/or symptoms of one or more metabolic disorders. The method can include administering to a subject having or at risk for developing a metabolic disorder a therapeutically effective amount of one or more anti-CGRP antagonist antibodies. The anti-CGRP antagonist antibody can be an antibody that binds CGRP. In some cases, a sub-therapeutic amount of a second agent for treating a metabolic disorder or aging that is known in the art can be co-administered with the disclosed compositions. The disclosed compositions and methods can also be useful in reducing side effects of the second agent.

CGRP has been noted for its possible connection to vasomotor symptoms (Wyon et al., Scand. J. Urol. Nephrol. 35: 92-96 (2001); Wyon et al., Menopause 7(1):25-30 (2000)). Vasomotor symptoms (VMS), such as hot flushes and night sweats, are the most common symptoms associated with menopause, occurring in 60% to 80% of all women following natural or surgically-induced menopause. Hot flushes are likely to be an adaptive response of the central nervous system (CNS) to declining sex steroids (Freedman, Am. J. Human Biol. 13:453-464 (2001)). Men also experience hot flushes following steroid hormone (androgen) withdrawal. This is true in cases of age-associated androgen decline (Katovich et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54). As many as one-third of these patients will experience persistent and frequent symptoms severe enough to cause significant discomfort and inconvenience.

CGRP is a potent vasodilator that has been implicated in the pathology of other vasomotor symptoms, such as all forms of vascular headache, including migraines (with or without aura) and cluster headache. Durham, N. Engl. J. Med. 350:1073-1075, 2004. The serum levels of CGRP in the external jugular vein are elevated in patients during migraine headache. Goadsby et al., Ann. Neurol. 28:183-7, 1990. Intravenous administration of human α-CGRP induced headache and migraine in patients suffering from migraine without aura, suggesting that CGRP has a causative role in migraine. Lassen et al., Cephalalgia 22:54-61, 2002. CGRP's involvement in migraine has been the basis for the development and testing of a number of compounds that inhibit release of CGRP (e.g., sumatriptan), antagonize at the CGRP receptor (e.g., dipeptide derivative BIBN4096BS (Boerhringer Ingelheim); CGRP(8-37)), or interact with one or more of receptor-associated proteins, such as, receptor activity membrane protein (RAMP) or receptor component protein (RCP), both of which affect binding of CGRP to its receptors. Brain et al., Trends in Pharmacological Sciences 23:51-53, 2002. Alpha-2 adrenoceptor subtypes and adenosine A1 receptors also control (inhibit) CGRP release and trigeminal activation (Goadsby et al., Brain 125:1392-401, 2002). The adenosine A1 receptor agonist GR79236 (metrafadil), which has been shown to inhibit neurogenic vasodilation and trigeminal nociception in humans, may also have anti-migraine activity (Arulmani et al., Cephalalgia 25:1082-1090, 2005; Giffin et al., Cephalalgia 23:287-292, 2003.) Confounding this theory is the observation that treatment with compounds that exclusively inhibit neurogenic inflammation (e.g., tachykinin NK1 receptor antagonists) or trigeminal activation (e.g., 5HT1D receptor agonists) have been shown to be relatively ineffective as acute treatments for migraine, leading some investigators to question whether inhibiting release of CGRP is the primary mechanism of action of effective anti-migraine treatments. Arulmani et al., Eur. J. Pharmacol. 500:315-330, 2004. Anti-CGRP antagonist antibodies have been used for treating migraine or pain as described in U.S. Pat. No. 8,623,366 hereby incorporated by reference in its entirety.

Organisms have evolved receptors and their associated signaling pathways designated to sense, recognize and appropriately respond to potential harmful stimuli in a deleterious environment for survival. Neural receptors that detect noxious stimuli can trigger pain sensations in order to promote avoidance and protect the organism from further harm. The transient receptor potential cation channel subfamily V member 1 (TRPV1) is expressed in afferent sensory neurons that detect extremely high temperatures and painful stimuli in target tissues such as the dermal and epidermal layers of the skin, the oral and nasal mucosa and joints. The cell bodies of these neurons relay information regarding environmental stimuli to the central nervous system via projection to the dorsal horn of the spinal cord. In addition to their diminished pain sensitivity, TRPV1 null mutant mice can show a phenotype that is their protection against diet-induced obesity. TRPV1 may have a role in metabolism. Unmyelinated C-fibers expressing TRPV1 form a dense meshwork innervating the pancreas, and their stimulation causes the release of neuropeptides, substance P and calcitonin gene related peptide (CGRP), which promote either neurogenic inflammation or antagonize insulin release in in vivo assays, respectively. The relationship between the release of CGRP, aging and the overall health and metabolism of the organism, however, is not fully understood.

The experience of pain necessarily causes a stress to the organism, however what remains unknown is the extent to which the perception, rather than the experience of pain elicits long-term consequences for the organism. Clinical studies have observed a correlative decrease in lifespan and overall decrease in health in patients that experience chronic levels of pain. Furthermore, in invertebrates in which the aging process is strongly affected by environmental factors, alterations in canonical sensory perception can acutely influence normal aging. Although it has been shown that genetic manipulation of the CRTC1/CREB pathway modulates aging of C. elegans and that disruption of chemosensory perception extends lifespan in worms and flies, whether similar mechanisms regulate mammalian longevity were unknown. It is shown herein that genetic deletion of TRPV1, an ion channel critical for nociception, extends mouse and C. elegans lifespan by regulating the activity of CRTC1 in peripheral sensory neurons. TRPV1 mutant mice also have improved glucose tolerance and increased energy expenditure throughout aging, despite a mild insulin resistance. Except for the latter, these phenotypes could underlie the exceptional longevity of these mice. Improved energy expenditure throughout aging prevents systemic damage associated from fat storage and fat metabolism by ensuring a healthier transition between carbohydrate and fat metabolism. Consistent with improved energy expenditure, TRPV1 mutant mice present beneficial effects on glucose tolerance and resistance to obesity induced by high fat diet.

It is shown herein that TRPV channels function in sensory neurons as an evolutionary conserved system integrating multiple sensory inputs and transducing them into neuroendocrine signals that promote longevity by adjusting the metabolic activity through the CRTC1/CREB circuit. In particular, these data highlight the role of the neuropeptide CGRP as a critical neuroendocrine regulator of longevity in mammals and possible biomarker of predictive lifespan and healthspan. Interestingly, the extremely long-lived naked mole rat, which lives over 30 years, is naturally lacking CGRP in DRGs (Park et al., J. Comp. Neurol. 465, 104-120, 2003). It is proposed herein that the pharmacological manipulation of TRPV1 and/or CGRP can be not only useful for pain but also to improve glucose homeostasis and aging. Consistent with this idea, diets rich in capsaicin, which can over stimulate TRPV1 neurons and cause their death, have long been linked to lower incidents of diabetes and metabolic dysregulation in humans (Westerterp-Plantenga et al., Int. J. Obes. 2005 29, 682-688, 2005).

The methods described herein include administering to a subject suffering (or at risk for) a metabolic disorder a composition that includes a therapeutically effective amount of one or more an anti-CGRP antagonist antibodies (such as 1, 2, 3, 4, or 5 different anti-CGRP antagonist antibodies). Exemplary subjects include animals, such as a mammal, for example a human. The methods and compositions described herein can be useful in both human therapeutics, pre-clinical, and veterinary applications. In some embodiments, the subject is a mammal, and in some embodiments, the subject is human. The subject treated is not limited by the age of the subject. For example, a human subject can be a child (e.g., a neonate, an infant, a toddler, a preadolescent), an adolescent, a pubescent, or an adult (e.g., an early adult, a middle aged adult, a senior citizen). In some embodiments, the human subject is a senior citizen. The human subject can be between about 0 months and about 120 years old, or older. For example, the human subject can be between about 0 and about 12 months old; between about 0 and 12 years old; between about 13 years and 19 years old (for example, about 13, 14, 15, 16, 17, 18, or 19 years old); between about 20 and about 39 year old (for example, about 20, 21, 22, 23, 24, 25, 26, 27, 28, 29, 30, 31, 32, 33, 34, 35, 36, 37, 38, or 39 years old); between about 40 to about 59 years old (for example, about 40, 41, 42, 43, 44, 45, 46, 47, 48, 49, 50, 51, 52, 53, 54, 55, 56, 57, 58, or 59 years old); or even greater than 59 years old (for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old). In specific embodiments, the human subject is about 45 years old or older; or 55 years old or older. The human subject can be a male subject or a female subject.

The subject can be on different types of diet that can induce a metabolic disorder. “Diet” refers to the types of food that a subject habitually consumes for a period of time. The period of time may be more than 1, 2, 3, 4, 5, 6, 7, 8, 9, 10, 11 or 12 months; 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 years. In some embodiments, the subject on some types of diet may be at risk of a metabolic disorder. In some embodiments, the subject can be on a high-sugar diet, high-fat diet, high-protein diet, low-fiber diet or a diet that is high in refined-starch. For each type of diet, “high” refers to a content level that is generally higher than the content in a normal diet that is well known in the art, as described by Food and Drug Administration (“Dietary Guidelines for Americans”, 2010). The subject may be on a diet that can cause elevation of one or more metabolic disorder risk factor levels. The subject may consume alcohol excessively. The subject may be on a diet that is high in dessert. The subject may be on a diet that is high in processed food.

The subjects treated can have different body weights. The body weight may be indicative of or associated with a metabolic disorder. The subject may be overweight or obese. Being overweight or obese means the body weight of the subject is above an ideal weight range. In general, an ideal weight range is about 18-25 in body mass index (BMI). The BMI is calculated by the weight of the subject in kilograms divided by the square of the height of the subject in meters. The subject may have a BMI that is about 25-30. For example, the subject may have a BMI that is about 25, 25.5, 26, 26.5, 27, 27.5, 28, 28.5, 29, 29.5, 29.9 or 30. The subject may have a BMI that is higher than about 30. For example, the subject may have a BMI that is about 30.5, 31, 32, 33, 34, 35, 36, 37, 38, 39 or 40. The subject may have a BMI that is higher than about 40. The subject may weight more than about 50, 75, 100, 125, 150, 160, 170, 180, 190, 200, 210, 220, 230, 240,250, 260, 270, 280, 290, 300, 310, 320, 330, 340, 350, 360, 370, 380, 390, or 400 lbs.

The subject may have a habit or may lack a habit. Such habit or lack of habit may be associated with a metabolic disorder and may put the subject at risk of developing the metabolic disorder. “Habit” refers to an activity that is performed by a subject routinely or in a frequency that is higher than normal. In some embodiments, the habit can increase glucose level in the bloodstream or serum, or inhibit insulin secretion in the subject. The subject may have a habit of smoking or inhaling smoke. The subject may have a habit of smoking tobacco products, including but are not limited to cigarettes, cigars, or pipes. The subject may be in an environment that contains smoke or second-hand smoke. The subject may not have a habit of exercising or performing physical activities regularly. The subject may exercise or perform physical activities in a frequency that is lower than normal. The subject may exercise, on average, about once in a week, a month, 2, 3, 4, 5, 6, 7, 8, 9, 10, 11, 12 months, 2, 3, 4, 5, 6, 7, 8, 9, 10 years. The subject may exercise less than about once in a week; once a month; once every 2, 3, 4, 5, 6, 7, 8, 9, 10, 11, 12 months; or once every 2, 3, 4, 5, 6, 7, 8, 9, 10 years. The subject may be immobilized.

The compositions and methods that include a therapeutically effective amount of anti-CGRP antagonist antibody as described herein can be used to treat a subject that is suffering from or at risk at suffering from a condition. The condition that the subject can be suffering from or at risk of suffering from can be a metabolic disorder or a condition that is associated with metabolic disorder. In some embodiments, the condition is diabetes (e.g., type I or II), liver disorder, pancreatic disorder, pregnancy, cardiovascular diseases, hyperlipidemia, obesity, weight gain, aging, and/or drug associated changes in lipid metabolism such as alcohol consumption, estrogen administration and the like. In some embodiments, the cardiovascular disease is coronary artery disease, stroke, hypertension, peripheral vascular disease, heart attack or ischemic heart disease, and/or congenital heart disease. Other exemplary metabolic disorders that a subject can be suffering from or at risk for include, but are not limited to: Acid Lipase Disease, Barth Syndrome, Central Pontine Myelinolysis, Farber's Disease, Gangliosidoses, lysosomal storage diseases, Gaucher's disease, Hunter Syndrome, Hurler Syndrome, Trimethylaminuria, Lesch-Nyhan Syndrome, Lipid Storage Diseases, Metabolic Diseases of Muscle, Metabolic Myopathies, Mitochondrial Myopathies, Mucolipidoses, Mucopolysaccharidoses, Niemann-Pick disease, Pompe Disease, Type I Glycogen Storage Disease, Sandhoff s disease, Tay-Sachs disease, Pompe Disease, Urea Cycle Disease, Hyperoxaluria and Oxalosis, and the like. Other examples are provided herein.

In one aspect, the subject methods and compositions can be used to treat a subject that is suffering from diabetes. Diabetes mellitus (DM) or diabetes, is a group of metabolic diseases characterized by an excessive level of glucose in the bloodstream, also known as hyperglycemia. Diabetes is one of the major diseases that endangers the public health due to its prevalence, morbidity and mortality. In recent years, the onset of diabetes or associated conditions has appeared in younger patients. Clinical symptoms of diabetes can be polyphagia, polydipsia, polyuria, fatigue, weight loss, blurred vision, poor wound healing, dry mouth, dry or itchy skin, tingling in feet or heels, erectile dysfunction, recurrent infections, external ear infections, cardiac arrhythmia, stupor, coma or seizures. Diabetes can further lead to hypertension, hyperlipidemia, coronary heart disease, chronic renal failure and other complications.

Diabetes can be categorized into two types: type I diabetes and type II diabetes. Type I diabetes is caused by the functional decline of the pancreatic β-cells. Therefore, type I diabetes is a disease caused by the lack of insulin. Type I diabetes patients are prone to acute complications, such as ketoacidosis. Type II diabetes is a complex metabolic disorder with different level of pathological symptoms. It can be characterized by the decline of islet β-cell functions, insulin resistance and glycogen metabolism disorders. Thus, the metabolic disorder that the subject can be suffering from (or at risk for) and can be treated by the subject compositions and methods can be type I or type II diabetes, such as type II diabetes.

The condition that the subject can be suffering from (or at risk for) can be a disorder that is characterized by inhibition of insulin secretion or high blood sugar (i.e., hyperglycemia, a high level or excessive amount of glucose circulating in the bloodstream, plasma or serum). Hyperglycemia can be characterized by a fasting glucose level that is higher than 100 mg/dl, but symptoms may not start to become noticeable until even higher values such as 250-300 mg/dl. A subject with a consistent range between 100 and 126 mg/dl (American Diabetes Association guidelines) is considered hyperglycemic, while above 126 mg/dl or 7 mmol/l is generally held to have diabetes or hyperglycemia. A subject with a consistent range below 70 mg/dl or 4 mmol/l can be considered hypoglycemic. Chronic levels exceeding 7 mmol/l (125 mg/dl) can produce organ damage. Glucose levels can vary before and after meals, and at various times of day. In general, the normal range of a fasting glucose level for most people can be about 80 to 110 mg/dl. Fasting glucose level generally refers to a serum glucose level that is measured after 8 to 12 hours of fasting, or fasting for 5 hours, 8 hours, 10 hours, 12 hours, 15 hours, 24 hours, or even longer. In fasting adults, normal blood plasma glucose can be lower than 126 mg/dL. Sustained higher levels of blood sugar can cause damage to the blood vessels and to the organs they supply, leading to the complications of diabetes. Hyperglycemia can be measured using any methods known in the medical art including the HbA1c test or glucose tolerance test. Thus, the condition that the subject is suffering from or at risk of suffering from can be a condition that is associated with an excessive serum level of glucose in the subject (such as a subject having a glucose level above 126 mg/dl, such as above 200 mg/dl or even above 300 mg/dl). A composition including one or more anti-CGRP antagonist antibodies can be used to change the serum level of glucose in the subject. The antibody can be an antibody that binds CGRP.

In some embodiments, the glucose levels of a subject are lowered following administration of one or more anti-CGRP antagonist antibodies, such as a reduction of at least 5%, at least 10%, at least 20%, at least 30%, at least 40%, at least 50%, or even at least 60%, as compared to a time prior to administration of the therapeutic composition. The glucose levels that are lowered may be fasting glucose level or glucose level after a meal, or during a glucose tolerance test. Glucose tolerance test or glucose challenge test is a medical test in which glucose is given and blood samples taken afterward to determine how quickly it is cleared from the blood. The test can be used to test for diabetes, insulin resistance, and sometimes reactive hypoglycemia and acromegaly, or rarer disorders of carbohydrate metabolism. The glucose tolerance test can be performed as an oral glucose tolerance test (OGTT), which includes ingesting a standard dose of glucose by mouth and checking the blood levels two hours later. Many variations of the glucose tolerance test have been devised over the years for various purposes, with different standard doses of glucose, different routes of administration, different intervals and durations of sampling, and various substances measured in addition to blood glucose.

In some examples, insulin tolerance in a subject are increased following administration of one or more anti-CGRP antagonist antibodies, such as an increase of at least 5%, at least 10%, at least 20%, at least 30%, at least 40%, at least 50%, at least 75%, at least 100%, or even at least 200%, as compared to a control, such as a time prior to administration of the therapeutic composition. Methods of measuring insulin tolerance are known in the art, and examples are provided herein.

In some examples, the life span of a subject is increased following administration of one or more anti-CGRP antagonist antibodies, such as an increase of at least 1 month, at least 6 months, at least 1 year, at least 2 years, at least 3 years, at least 4 years, at least 5 years, or at least 10 years, as compared to a control, such as compared to the life span of a subject or group of subjects having the same metabolic disorder, but who did not receive the therapeutic composition.

In some examples, the respiratory exchange ratio (RER) in a subject is increased following administration of one or more anti-CGRP antagonist antibodies, such as an increase of at least 5%, at least 10%, at least 20%, at least 30%, at least 40%, at least 50%, at least 75%, at least 100%, or even at least 200%, as compared to a control, such as a time prior to administration of the therapeutic composition or as compared to the RER of a subject or group of subjects having the same metabolic disorder, but who did not receive the therapeutic composition. Methods of measuring RER are known in the art, and examples are provided herein.

In some examples, the oxygen consumption in a subject is increased following administration of one or more anti-CGRP antagonist antibodies, such as an increase of at least 5%, at least 10%, at least 20%, at least 30%, at least 40%, at least 50%, at least 75%, at least 100%, or even at least 200%, as compared to a control, such as a time prior to administration of the therapeutic composition or as compared to the oxygen consumption of a subject or group of subjects having the same metabolic disorder, but who did not receive the therapeutic composition. Methods of measuring oxygen consumption are known in the art, and examples are provided herein.

The subject that is suffering from (or at risk for) a metabolic disorder or a condition that is associated to a metabolic disorder can be diagnosed or selected using one or more tests that is known in the medical art. The methods or tests that can be used to identify, select or diagnose the subject that is suffering a metabolic disorder or at risk of suffering a metabolic disorder include, but are not limited to: levels of biomarkers selected from a metabolic panel (American Association for Clinical Chemistry), lactate test, methylmalonic acid test, mitochondrial disease symptoms, urine odor, glucose tolerance test, and the like.

In some embodiments, the methods and compositions including one or more anti-CGRP antagonist antibodies are useful for administering to a subject that is classified according to the guideline to be at a very high risk, high risk, borderline-high risk or near optimal on the serum glucose level for diabetes or a metabolic disorder. For example, the subject can have a fasting serum glucose level that is higher than 80, 85, 90, 95, 100, 105, 110, 115, 120, 125, 126, 130, 135, 140, 145, 150, 155, 160, 165, 170, 175, 180, 185, 190, 195, 200, 250, or 300 mg/dL. In some embodiments, anti-CGRP antagonist antibody can be administered to the subject that has fasting serum glucose level that is about 80-300, 80-250, 80-125, 80-100, 100-300, 100-250, 100-125, 125-300, or 250-300 mg/dL. In some embodiments, anti-CGRP antagonist antibody can be used on the subject that has a fasting serum glucose level that is higher than about 100, 125, or 250 mg/dL. Where desired, the selected physiological level of serum glucose after administration of the therapeutically effective amount of an anti-CGRP antagonist antibody may be measured under a fasting condition, e.g., without taking food for at least about 8 hours, 10 hours, 12 hours, 15 hours, 24 hours, or even longer or between 8-12 hours. In some cases, the serum glucose level after administration of the anti-CGRP antagonist antibody is measured 2 hours after ingestion of glucose, or after a meal.

Methods and compositions are described herein for administering a therapeutically effective amount of one or more anti-CGRP antagonist antibodies to a subject with a condition such that the condition of the subject is improved following the administration of the anti-CGRP antagonist antibody. The anti-CGRP antagonist antibody can be an antibody that binds CGRP. The administration of the therapeutically effective amount of anti-CGRP antagonist antibody may result in improvement of the condition by at least 5%, at least 10%, at least 15%, at least 20%, at least 25%, at least 30%, at least 35%, at least 40%, at least 5%, at least 50%, at least 55%, at least 60%, at least 65%, at least 70%, at least 75%, at least 80%, at least 85%, at least 90%, at least 95%, and at least 99%. In some embodiments, the condition is improved by greater than 50%. The improvement may be characterized with respect to a level of biomarkers or risk factors in the subject, such as a biomarker associated with the condition being treated. The improvement may also be characterized by the functional improvement of an organ in comparison to its normal function. The improvement may also be characterized by increased insulin secretion, increased glucose tolerance, increased longevity or overall metabolic health. Such improvement can be relative to the condition prior to such treatment, or relative to what is expected in a subject having or at risk for the same metabolic condition(s).

In some embodiments, the compositions and methods as described herein are useful for ameliorating or reducing one or more side-effects of an anti-metabolic disorder agent. Methods and compositions that include an anti-CGRP antagonist antibody described herein can also be administered to a subject that is also administered with one or more anti-metabolic disorder agents. In some embodiments, the one or more anti-metabolic disorder agents are administered in an amount effective to treat the metabolic disorder. In some embodiments, the one or more anti-metabolic disorder agents are administered in an amount effective to result in lowering glucose levels in the subject. In some embodiments, the one or more anti-metabolic disorder agents may have a side-effect. In another case, the one or more anti-hyperglycemic agents may be administered in a sub-therapeutic level in conjunction with the anti-CGRP antagonist antibody to minimize the side effects. The anti-CGRP antagonist antibody can be an antibody that binds CGRP.

In some embodiments, the subject can be further administered an effective amount of one or more therapeutic agents including anti-metabolic disorder agents or anti-diabetes agents. The methods described herein can thus include administrating therapeutic amounts of one or more anti-CGRP antagonist antibodies, and administering therapeutic amounts of one or more additional therapeutic agents (e.g., anti-metabolic disorder agents or anti-diabetes agents). In some embodiments, the anti-CGRP antagonist antibody is administered together with the one or more additional therapeutic agents at the same time in the same route. In some embodiments, the anti-CGRP antagonist antibody is administered separately from the one or more additional therapeutic agents. In some embodiments, the anti-CGRP antagonist antibody and the one or more additional therapeutic agents may be administered subsequently, with the additional therapeutic agent first or anti-CGRP antagonist antibody first. In some embodiments, the anti-CGRP antagonist antibody is administered to a subject in conjunction with one or more additional therapeutic agents. In some embodiments, the anti-CGRP antagonist antibody is administered by the same administration route with the one or more additional therapeutic agents. The anti-CGRP antagonist antibody may be administered by a different administration route with the one or more additional therapeutic agents. For example, the anti-CGRP antagonist antibody may be administered subcutaneously while the one or more additional therapeutic agents may be administered via intravenous injection. Each of the one or more additional therapeutic agents may be administered via the same or different administration routes.

One or more anti-CGRP antagonist antibodies (as well as the one or more additional therapeutic agents (e.g., anti-metabolic disorder agents or anti-diabetes agents)) can be administered in any suitable manner. Such administration may be subcutaneous injection. Other administration routes may be used, such as oral, transdermal, intravenous, aerosol, intramuscular, vaginal, rectal, subdermal, parenteral, ophthalmic, pulmonary, transmucosal, otic, nasal, and topical administration. Components of a composition described herein, such as an anti-CGRP antagonist antibody, at least one agent for increasing the bioavailability of the anti-CGRP antagonist antibody, or at least one additional therapeutic agent may be administered to a subject concurrently, such as in any manner of concurrent administration described herein and/or in U.S. Patent Application Publication No. US 2006/0089335 A1. In addition, by way of example only, parenteral delivery includes intramuscular, subcutaneous, intravenous, intramedullary injections, as well as intrathecal, direct intraventricular, intraperitoneal, intralymphatic, and intranasal injections.

In one embodiment, one or more anti-CGRP antagonist antibodies are administered via site-specific or targeted local delivery techniques. Examples of site-specific or targeted local delivery techniques include various implantable depot sources of the anti-CGRP antagonist antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Furthermore, in some cases, the methods described herein include administering the one or more anti-CGRP antagonist antibodies for the same or different duration of treatment as the one or more additional therapeutic agents. For example, the one or more anti-CGRP antagonist antibodies may be administered for 30 days while the one or more additional therapeutic agents are administered for 10 days. The subject may be on a treatment with one or more additional therapeutic agents for a period of time before administering the one or more anti-CGRP antagonist antibodies. The a one or more anti-CGRP antagonist antibodies and the one or more additional therapeutic agents may act upon the metabolic disorder synergistically.

Examples of the additional therapeutic agents that can be co-administered with the one or more anti-CGRP antagonist antibodies include anti-diabetes agents known in the art. The additional therapeutic agents may include, but are not limited to, one or more of (such as 1, 2, 3, 4, or 5 of) HMG-CoA inhibitors (or statins), biguanides, sufonylureas, thiazolidinediones, meglitinides glinides, α-glucosidase inhibitors, repaglinide, nateglinide, DPP-IV inhibitor, sitagliptin, vildagliptin, saxagliptin or insulin and insulin analogues. Non-limiting examples of insulin and insulin analogues for use to treat hyperglycemia or diabetes, in particular type I diabetes, can include rapid acting insulins, intermediate acting insulins and long acting insulins. The rapid acting insulin can include regular insulin (Humulin R, Novolin R), insulin lispro (Humalog), insulin aspart (Novolog), insulin glulisine (Apidra), prompt insulin zinc (Semilente) and the like. The intermediate acting insulins can include isophane insulin, neutral protamine hagedorn (Humulin N, Novolin N), insulin zinc (Lente) and the like. The long acting insulins can include extended insulin zinc insulin (Ultralente), insulin glargine (Lantus), insulin detemir (Levemir), and the like. The anti-hyperglycemic agents can be insulin sensitizers. Insulin sensitizers can be used to treat type II diabetes in particular. Exemplary insulin sensitizers include, but are not limited to: biguanides or thiazolidinediones. The biguanides can be Metformin, Phenformin or Buformin. Metformin is usually the first-line medication used for treatment of type II diabetes clinically. The thiazolidinediones can be rosiglitazone (Avandia), pioglitazone (Actos) or troglitazone (Rezulin). The anti-hyperglycemic agents can be agents that increase insulin output from pancreas, such as secretagogues. The secretagogues can be sulfonylureas, or nonsulfonylurea secretagogues. The sulfonylureas can include, but are not limited to: tolutamide (Orinase, Rastinon brand name), acetohexamide (Dymelor), tolazamide (Tolinase), chlorpropamide (Diabinase), glipizide (Glucotrol, Minidiab, Glibenese), glyburide or glibenclamide (Diabeta, Micronase, Glynase, Danil, Euglycon), glimepiride (Amaryl), gliclazide (Uni Diamicron), glycopyramide, gliquidone (Glurenorm). The nonsulfonylurea secretagogues can include meglitinides, repaglinide (Prandin, Novonorm), or nateglinide (Starlix). The anti-hyperglycemic agents or anti-diabetes agents can be alpha glucosidase inhibitors. Alpha glucosidase inhibitors can slow the digestion of starch in the small intestine, so that glucose from the starch of a meal can enter the bloodstream more slowly, and can be matched more effectively by an impaired insulin response or sensitivity. Non-limiting examples of alpha glucosidase inhibitors can include miglitol (Glyset), acarbose (Precose/Glucobay), or voglibose. The anti-hyperglycemic agents can be peptide analogs. The peptide analogs can include but are not limited to: injectable incretin mimetics, injectable glucagon-like peptide analogs and agonists, gastric inhibitory peptide analogs, dipeptidyl peptidase-4 inhibitors, or injectable amylin analogues. The injectable incretin mimetics can include glucagon-like peptide-1 (GLP-1) and gastric inhibitory peptide (glucose-dependent insulinotropic peptide, GIP). The injectable glucagon-like peptide analogs and agonists can include exenatide (also exendin-4 or Byetta), liraglutide, taspoglutide, or lixisenatide (Lyxumia) sanofi aventis. The dipeptidyl peptidase-4 inhibitors can increase blood concentration of the incretin GLP-1 by inhibiting its degradation by dipeptidyl peptidase-4. The dipeptidyl peptidase-4 inhibitors can include vildagliptin (Galvus), sitagliptin (Januvia), saxagliptin (Onglyza), linagliptin (Tradjenta), allogliptin or septagliptin. The injectable amylin analogues can be pramlintide. The anti-hyperglycemic or anti-diabetes agents can be glycosurics. The glycosurics can be sodium/glucose cotransporter 2 (SGLT2) and it functions to block the re-uptake of glucose in the renal tubules, promoting loss of glucose in the urine. The glycosurics can be canagliflozin (Invokana) or dapagliflozin (Forxiga). In some cases, the anti-hyperglycemic agents or anti-diabetes agents can be natural substances such as plants, elements or chinese medicine. Non-limiting examples of natural substances as anti-hyperglycemic or anti-diabetes agents include cinnamon, sandalwood, kino tree, Gentiana olivieri, chromium supplements, vanadyl sulfate or thiamine.

Other examples of additional therapeutic agents that can be co-administered with the one or more anti-CGRP antagonist antibodies include but are not limited to, an alpha-glucosidase inhibitor, an amylin agonist, a dipeptidyl-peptidase 4 (DPP-4) inhibitor, meglitinide, sulfonylurea, or a peroxisome proliferator-activated receptor (PPAR)-gamma agonist. Examples of PPAR-gamma agonists include a thiazolidinedione (TZD) (such as pioglitazone, rosiglitazone, rivoglitazone, or troglitazone), aleglitazar, farglitazar, muraglitazar, or tesaglitazar.

When the one or more therapeutic agents (e.g., anti-hyperglycemic agents or anti-diabetes agents) as described herein is administered with an anti-CGRP antagonist antibody, the one or more therapeutic agents can be used in an amount that is sub-therapeutic. The sub-therapeutic amount of a therapeutic agent or anti-diabetes agent as described herein can be about 1, 2, 3, 4, 5, 6, 7, 8, 9, 10, 15, 20, 25, 30, 35, 40, 45, 50, 55, 60, 65, 70, 75, 80, 85, 90, 95 or 99% less than the amount that is considered to be a therapeutic amount when used alone. The therapeutic amount can be a suggested dose by FDA guidelines, or an amount that is assessed for individual subjects or for a group of subjects all having one or more characteristics. The characteristics can be one or more of age, weight, race, gender, ethnicity, nationality, a particular diet, physical activity level or a level of certain biomarkers.

### Anti-CGRP Antagonist Antibodies

In some embodiments, the methods provided herein use one or more anti-CGRP antagonist antibodies.

An anti-CGRP antagonist antibody can exhibit any one or more of the following characteristics: (a) bind to CGRP; (b) block CGRP from binding to its receptor(s); (c) block or decrease CGRP receptor activation (including cAMP activation); (d) inhibit CGRP biological activity or downstream pathways mediated by CGRP signaling function; (e) prevent, ameliorate, or treat any aspect of headache (e.g., migraine); (f) increase clearance of CGRP; and (g) inhibit (reduce) CGRP synthesis, production or release. Some anti-CGRP antagonist antibodies are known in the art. See, e.g., Tan et al., Clin. Sci. (Lond). 89:565-73, 1995; Sigma (Missouri, US), product number C7113 (clone #4901); Plourde et al., Peptides 14:1225-1229, 1993.

In some embodiments, the anti-CGRP antagonist antibody reacts with CGRP in a manner that inhibits CGRP and/or downstream pathways mediated by the CGRP signaling function. In some embodiments, the anti-CGRP antagonist antibody recognizes human CGRP (such as SEQ ID NO: 15). In some embodiments, the anti-CGRP antagonist antibody binds to both human α-CGRP and β-CGRP (e.g., SEQ ID NOS: 15 and 43). In some embodiments, the anti-CGRP antagonist antibody binds human and rat CGRP (e.g., SEQ ID NOS: 15 and 41, 43 and 44, or all four of these sequences). In some embodiments, the anti-CGRP antagonist antibody binds the C-terminal fragment having amino acids 25-37 of CGRP (e.g., SEQ ID NO: 25). In some embodiments, the anti-CGRP antagonist antibody binds a C-terminal epitope within amino acids 25-37 of CGRP (e.g., within SEQ ID NO: 25).

The antibodies useful in the present disclosure include monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, camelid antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the anti-CGRP antagonist antibody is a monoclonal antibody. In some embodiments, the anti-CGRP antagonist antibody is humanized. In some embodiments, the antibody is human. In some embodiments, the anti-CGRP antagonist antibody is antibody G1 (as described herein). In some embodiments, the anti-CGRP antagonist antibody includes one or more CDR(s) (such as one, two, three, four, five, or, in some embodiments, all six CDRs) of antibody G1 or variants of G1 shown in Table 6. In still other embodiments, the anti-CGRP antagonist antibody includes the amino acid sequence of the heavy chain variable region shown in FIG. 5 (SEQ ID NO: 1) and the amino acid sequence of the light chain variable region shown in FIG. 5 (SEQ ID NO: 2).

In some embodiments, the anti-CGRP antagonist antibody comprises a modified constant region, such as a constant region that is immunologically inert, as described herein. In some embodiments, the constant region is modified as described in Eur. J. Immunol. (1999) 29:2613-2624; PCT Application No. PCT/GB99/01441; and/or UK Patent Application No. 9809951.8. In other embodiments, the antibody includes a human heavy chain IgG2 constant region comprising the following mutations: A330P331 to S330S331 (amino acid numbering with reference to the wildtype IgG2 sequence). Eur. J. Immunol. (1999) 29:2613-2624. In some embodiments, the antibody comprises a constant region of IgG4 comprising the following mutations: E233F234L235 to P233V234A235. In still other embodiments, the constant region is aglycosylated for N-linked glycosylation. In some embodiments, the constant region is aglycosylated for N-linked glycosylation by mutating the oligosaccharide attachment residue (such as Asn297) and/or flanking residues that are part of the N-glycosylation recognition sequence in the constant region. In some embodiments, the constant region is aglycosylated for N-linked glycosylation. The constant region may be aglycosylated for N-linked glycosylation enzymatically or by expression in a glycosylation deficient host cell.

The binding affinity (K_{D}) of an anti-CGRP antagonist antibody to CGRP (such as human α-CGRP) can be about 0.02 to about 200 nM. In some embodiments, the binding affinity is any of about 200 nM, about 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, or about 50 pM.

One way of determining or measuring binding affinity of antibodies to CGRP is by measuring binding affinity of monofunctional Fab fragments of the antibody. To obtain monofunctional Fab fragments, an antibody (for example, IgG) can be cleaved with papain or expressed recombinantly. The affinity of an anti-CGRP Fab fragment of an antibody can be determined by surface plasmon resonance (Biacore3000™ surface plasmon resonance (SPR) system, Biacore, INC, Piscataway N.J.) equipped with pre-immobilized streptavidin sensor chips (SA) using HBS-EP running buffer (0.01M HEPES, pH 7.4, 0.15 NaCl, 3 mM EDTA, 0.005% v/v Surfactant P20). Biotinylated human CGRP (or any other CGRP) can be diluted into HBS-EP buffer to a concentration of less than 0.5 ug/mL and injected across the individual chip channels using variable contact times, to achieve two ranges of antigen density, either 50-200 response units (RU) for detailed kinetic studies or 800-1,000 RU for screening assays. Regeneration studies have shown that 25 mM NaOH in 25% v/v ethanol effectively removes the bound Fab while keeping the activity of CGRP on the chip for over 200 injections. Typically, serial dilutions (spanning concentrations of 0.1-10× estimated K_{D}) of purified Fab samples are injected for 1 min at 100 μL/minute and dissociation times of up to 2 hours are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data globally to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values are calculated as k_{off}/kₒₙ. This protocol is suitable for use in determining binding affinity of an antibody to any CGRP, including human CGRP, CGRP of another mammalian (such as mouse CGRP, rat CGRP, primate CGRP), as well as different forms of CGRP (such as α and β form). Binding affinity of an antibody is generally measured at 25° C., but can also be measured at 37° C.

The anti-CGRP antagonist antibodies may be made by any method known in the art. The route and schedule of immunization of the host animal are generally in keeping with established and conventional techniques for antibody stimulation and production, as further described herein. General techniques for production of human and mouse antibodies are known in the art and are described herein.

It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck et al., In Vitro, 18:377-381 (1982). Available myeloma lines, including but not limited to X63-Ag8.653 and those from the Salk Institute for Biological Studies, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-CGRP monoclonal antibodies of the subject disclosure. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for CGRP, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a human CGRP, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaradehyde, succinic anhydride, SOCl2, or R1N═C═NR, where R and R1 are different alkyl groups, can yield a population of antibodies (e.g., monoclonal antibodies).

If desired, the anti-CGRP antagonist antibody (monoclonal or polyclonal) of interest may be sequenced and the corresponding polynucleotide sequence may then be cloned into a vector for expression or propagation. The sequence encoding the antibody of interest may be maintained in a vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to CGRP and greater efficacy in inhibiting CGRP. It will be apparent to one of skill in the art that one or more polynucleotide changes can be made to the anti-CGRP antagonist antibody and still maintain its binding ability to CGRP.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al., Proc. Nat. Acad. Sci. USA 86:4220-4224 (1989), Shaw et al., J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al., Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g. PCT Publication No. PCT/GB99/01441; UK Patent Application No. 9809951.8. Other methods of humanizing antibodies that may also be utilized are disclosed by Daugherty et al., Nucl. Acids Res. 19:2471-2476 (1991) and in U.S. Pat. Nos. 6,180,377; 6,054,297; 5,997,867; 5,866,692; 6,210,671; and 6,350,861; and in PCT Publication No. WO 01/27160.

In some embodiments, fully human antibodies may be obtained by using commercially available mice that have been engineered to express specific human immunoglobulin proteins. Transgenic animals that are designed to produce a desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In some embodiments, antibodies may be made recombinantly and expressed using any method known in the art. In some embodiments, antibodies may be made recombinantly by phage display technology. See, for example, U.S. Pat. Nos. 5,565,332; 5,580,717; 5,733,743; and 6,265,150; and Winter et al., Annu. Rev. Immunol. 12:433-455 (1994). The phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson and Chiswell, Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin.

Although the above discussion pertains to humanized antibodies, the general principles discussed are applicable to customizing antibodies for use, for example, in dogs, cats, primate, equines and bovines. It is further apparent that one or more aspects of humanizing an antibody described herein may be combined, e.g., CDR grafting, framework mutation and CDR mutation.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters et al. Vaccine 19:2756 (2001); Lonberg and Huszar Int. Rev. Immunol 13:65 (1995); and Pollock et al., J Immunol Methods 231:147(1999). Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for CGRP.

The disclosed antibodies can be bound to many different carriers. Carriers can be active and/or inert. Examples of well-known carriers that can be used include but are not limited to polypropylene, polystyrene, polyethylene, dextran, nylon, amylases, glass, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation. In some embodiments, the carrier comprises a moiety that targets the myocardium.

DNA encoding the monoclonal antibodies can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-CGRP monoclonal antibody herein.

Anti-CGRP antagonist antibodies and polypeptides derived from antibodies can be identified or characterized using methods known in the art, whereby reduction, amelioration, or neutralization of an CGRP biological activity is detected and/or measured. For example, anti-CGRP antagonist antibody can also be identified by incubating a candidate agent with CGRP and monitoring any one or more of the following characteristics: (a) bind to CGRP; (b) block CGRP from binding to its receptor(s); (c) block or decrease CGRP receptor activation (including cAMP activation); (d) inhibit CGRP biological activity or downstream pathways mediated by CGRP signaling function; (e) prevent, ameliorate, or treat any aspect of headache (e.g., migraine); (f) increase clearance of CGRP; and (g) inhibit (reduce) CGRP synthesis, production or release. In some embodiments, an anti-CGRP antagonist antibody or polypeptide is identified by incubating a candidate agent with CGRP and monitoring binding and/or attendant reduction or neutralization of a biological activity of CGRP. The binding assay may be performed with purified CGRP polypeptide(s), or with cells naturally expressing, or transfected to express, CGRP polypeptide(s). In one embodiment, the binding assay is a competitive binding assay, where the ability of a candidate antibody to compete with a known anti-CGRP antagonist for CGRP binding is evaluated. The assay may be performed in various formats, including the ELISA format. In other embodiments, an anti-CGRP antagonist antibody is identified by incubating a candidate agent with CGRP and monitoring binding and attendant inhibition of CGRP receptor activation expressed on the surface of a cell.

Following initial identification, the activity of a candidate anti-CGRP antagonist antibody can be further confirmed and refined by bioassays, known to test the targeted biological activities. Alternatively, bioassays can be used to screen candidates directly. For example, CGRP promotes a number of measurable changes in responsive cells. These include, but are not limited to, stimulation of cAMP in the cell (e.g., SK-N-MC cells). Antagonist activity may also be measured using animal models, such as measuring skin vasodilatation induced by stimulation of the rat saphenous nerve. Escott et al., Br. J. Pharmacol. 110: 772-776, 1993. Animal models of headaches (such as, migraine) may further be used for testing efficacy of antagonist antibodies or polypeptides. Reuter et al., Functional Neurology (15) Suppl. 3, 2000. Some of the methods for identifying and characterizing anti-CGRP antagonist antibody or polypeptide are described in detail in the Examples.

Anti-CGRP antagonist antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-CGRP antagonist antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-CGRP antagonist antibody. In another example, the epitope to which the anti-CGRP antagonist antibody binds can be determined in a systematic screening by using overlapping peptides derived from the CGRP sequence and determining binding by the anti-CGRP antagonist antibody. According to the gene fragment expression assays, the open reading frame encoding CGRP is fragmented either randomly or by specific genetic constructions and the reactivity of the expressed fragments of CGRP with the antibody to be tested is determined. The gene fragments may, for example, be produced by PCR and then transcribed and translated into protein in vitro, in the presence of radioactive amino acids. The binding of the antibody to the radioactively labeled CGRP fragments is then determined by immunoprecipitation and gel electrophoresis. Certain epitopes can also be identified by using large libraries of random peptide sequences displayed on the surface of phage particles (phage libraries). A defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant CGRP in which various fragments of the CGRP polypeptide have been replaced (swapped) with sequences from a closely related, but antigenically distinct protein (such as another member of the neurotrophin protein family). By assessing binding of the antibody to the mutant CGRP, the importance of the particular CGRP fragment to antibody binding can be assessed.

Yet another method which can be used to characterize an anti-CGRP antagonist antibody is to use competition assays with other antibodies known to bind to the same antigen, i.e., various fragments on CGRP, to determine if the anti-CGRP antagonist antibody binds to the same epitope as other antibodies. Competition assays are well known to those of skill in the art.

An expression vector can be used to direct expression of an anti-CGRP antagonist antibody. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventrical, or pericardium.

Targeted delivery of therapeutic compositions containing an expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

### Antibody G1 and Related Antibodies, Polypeptides, Polynucleotides, Vectors and Host Cells

Disclosed herein are compositions, including pharmaceutical compositions, comprising antibody G1 and its variants shown in Table 6 or polypeptide derived from antibody G1 and its variants shown in Table 6; and polynucleotides comprising sequences encoding G1 and its variants or the polypeptide. In some embodiments, the compositions include one or more antibodies or polypeptides (which may or may not be an antibody) that bind to CGRP, and/or one or more polynucleotides comprising sequences encoding one or more antibodies or polypeptides that bind to CGRP. These compositions may further comprise suitable excipients, such as pharmaceutically acceptable excipients including buffers, which are well known in the art.

The anti-CGRP antagonist antibodies and polypeptides of the disclosure are characterized by any (one or more) of the following characteristics: (a) bind to CGRP; (b) block CGRP from binding to its receptor(s); (c) block or decrease CGRP receptor activation (including cAMP activation); (d) inhibit CGRP biological activity or downstream pathways mediated by CGRP signaling function; (e) prevent, ameliorate, or treat any aspect of headache (e.g., migraine); (f) increase clearance of CGRP; and (g) inhibit (reduce) CGRP synthesis, production or release.

Accordingly, the disclosure provides any of the following, or compositions (including pharmaceutical compositions) including any of the following: (a) antibody G1 or its variants shown in Table 6; (b) a fragment or a region of antibody G1 or its variants shown in Table 6; (c) a light chain of antibody G1 or its variants shown in Table 6; (d) a heavy chain of antibody G1 or its variants shown in Table 6; (e) one or more variable region(s) from a light chain and/or a heavy chain of antibody G1 or its variants shown in Table 6; (f) one or more CDR(s) (one, two, three, four, five or six CDRs) of antibody G1 or its variants shown in Table 6; (g) CDR H3 from the heavy chain of antibody G1; (h) CDR L3 from the light chain of antibody G1 or its variants shown in Table 6; (i) three CDRs from the light chain of antibody G1 or its variants shown in Table 6; (j) three CDRs from the heavy chain of antibody G1 or its variants shown in Table 6; (k) three CDRs from the light chain and three CDRs from the heavy chain, of antibody G1 or its variants shown in Table 6; and (1) an antibody comprising any one of (b) through (k). The disclosure also provides polypeptides including any one or more of the above.

The CDR portions of antibody G1 (including Chothia and Kabat CDRs) are diagrammatically depicted in FIG. 5. Determination of CDR regions is well within the skill of the art. It is understood that in some embodiments, CDRs can be a combination of the Kabat and Chothia CDR (also termed “combined CDRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

In some embodiments, the disclosure provides a polypeptide (which may or may not be an antibody) which comprises at least one CDR, at least two, at least three, or at least four, at least five, or all six CDRs that are substantially identical to at least one CDR, at least two, at least three, at least four, at least five or all six CDRs of G1 or its variants shown in Table 6. Other embodiments include antibodies which have at least two, three, four, five, or six CDR(s) that are substantially identical to at least two, three, four, five or six CDRs of G1 or derived from G1. In some embodiments, the at least one, two, three, four, five, or six CDR(s) are at least about 85%, 86%, 87%, 88%, 89%, 90%, 95%, 96%, 97%, 98%, or 99% identical to at least one, two, three, four, five or six CDRs of G1 or its variants shown in Table 6. It is understood that, for purposes of this disclosure, binding specificity and/or overall activity is generally retained, although the extent of activity may vary compared to G1 or its variants shown in Table 6 (may be greater or lesser).

The disclosure also provides a polypeptide (which may or may not be an antibody) which includes an amino acid sequence of G1 or its variants shown in Table 6 that has any of the following: at least 5 contiguous amino acids, at least 8 contiguous amino acids, at least about 10 contiguous amino acids, at least about 15 contiguous amino acids, at least about 20 contiguous amino acids, at least about 25 contiguous amino acids, at least about 30 contiguous amino acids of a sequence of G1 or its variants shown in Table 6, wherein at least 3 of the amino acids are from a variable region of G1 (FIG. 5) or its variants shown in Table 6. In one embodiment, the variable region is from a light chain of G1. In another embodiment, the variable region is from a heavy chain of G1. An exemplary polypeptide has contiguous amino acid (lengths described above) from both the heavy and light chain variable regions of G1. In another embodiment, the 5 (or more) contiguous amino acids are from a complementarity determining region (CDR) of G1 shown in FIG. 5. In some embodiments, the contiguous amino acids are from a variable region of G1.

The binding affinity (K_{D}) of an anti-CGRP antagonist antibody and polypeptide to CGRP (such as human α-CGRP) in some examples can be about 0.06 to about 200 nM. In some embodiments, the binding affinity is any of about 200 nM, 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, or about 50 pM.

The disclosure also provides methods of making any of these antibodies or polypeptides. The antibodies can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In some embodiments, the antibodies can be made recombinantly using procedures that are well known in the art. In one embodiment, a polynucleotide comprises a sequence encoding the heavy chain and/or the light chain variable regions of antibody G1 shown in SEQ ID NO:9 and SEQ ID NO:10. In another embodiment, the polynucleotide comprising the nucleotide sequence shown in SEQ ID NO:9 and SEQ ID NO:10 are cloned into one or more vectors for expression or propagation. The sequence encoding the antibody of interest may be maintained in a vector in a host cell and the host cell can then be expanded and frozen for future use. Vectors (including expression vectors) and host cells are further described herein.

The disclosure also encompasses single chain variable region fragments (“scFv”) of antibodies of this disclosure, such as G1. Single chain variable region fragments are typically made by linking light and/or heavy chain variable regions by using a short linking peptide. Bird et al. (1988) Science 242:423-426. An example of a linking peptide is (GGGGS)3 (SEQ ID NO: 57) which bridges approximately 3.5 nm between the carboxy terminus of one variable region and the amino terminus of the other variable region. Linkers of other sequences have been designed and used. Bird et al. (1988). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad Sci. USA 90:6444-6448; Poljak et al. (1994) Structure 2:1121-1123).

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. In one example, the first heavy chain constant region (CH1), containing the site necessary for light chain binding, is present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the disclosure. Such antibodies have been used to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Humanized antibody comprising one or more CDRs of antibody G1 or its variants shown in Table 6, or one or more CDRs derived from antibody G1 or its variants shown in Table 6 can be made using any methods known in the art. For example, four general steps may be used to humanize a monoclonal antibody.

The disclosure encompasses modifications to antibody G1 or its variants shown in Table 6, including functionally equivalent antibodies which do not significantly affect their properties and variants which have enhanced or decreased activity and/or affinity. For example, the amino acid sequence of antibody G1 or its variants shown in Table 6 may be mutated to obtain an antibody with the desired binding affinity to CGRP. Modification of polypeptides is routine practice in the art and need not be described in detail herein. Modification of polypeptides is exemplified in the Examples. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 1 under the heading of “conservative substitutions”. If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) Non-polar: Norleucine, Met, Ala, Val, Leu, Ile;

(2) Polar without charge: Cys, Ser, Thr, Asn, Gln;

(3) Acidic (negatively charged): Asp, Glu;

(4) Basic (positively charged): Lys, Arg;

(5) Residues that influence chain orientation: Gly, Pro; and

(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDR H3 and/or CDR L3.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies, such as anti-CGRP antagonist antibodies, is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified G1 polypeptides are made using established procedures in the art and can be screened using standard assays known in the art, some of which are described below and in the Examples.

In some embodiments, the anti-CGRP antagonist antibody includes a modified constant region, such as a constant region that is immunologically inert or partially inert, e.g., does not trigger complement mediated lysis, does not stimulate antibody-dependent cell mediated cytotoxicity (ADCC), or does not activate microglia; or have reduced activities (compared to the unmodified antibody) in any one or more of the following: triggering complement mediated lysis, stimulating antibody-dependent cell mediated cytotoxicity (ADCC), or activating microglia. Different modifications of the constant region may be used to achieve optimal level and/or combination of effector functions. See, for example, Morgan et al., Immunology 86:319-324 (1995); Lund et al., J. Immunology 157:4963-9 157:4963-4969 (1996); Idusogie et al., J. Immunology 164:4178-4184 (2000); Tao et al., J. Immunology 143: 2595-2601 (1989); and Jefferis et al., Immunological Reviews 163:59-76 (1998). In some embodiments, the constant region is modified as described in Eur. J. Immunol. (1999) 29:2613-2624; PCT Application No. PCT/GB99/01441; and/or UK Patent Application No. 9809951.8. In other embodiments, the anti-CGRP antagonist antibody includes a human heavy chain IgG2 constant region comprising the following mutations: A330P331 to S330S331 (amino acid numbering with reference to the wildtype IgG2 sequence). Eur. J. Immunol. (1999) 29:2613-2624. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601 (1989); and Jefferis et al., Immunological Reviews 163:59-76 (1998). In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO 99/58572, published Nov. 18, 1999. These antibodies can include, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain C_{H}2 domains. Antibodies, such as anti-CGRP antagonist antibodies, modified in this manner are particularly suitable for use in chronic antibody therapy, to avoid inflammatory and other adverse reactions to conventional antibody therapy.

The disclosure includes affinity matured embodiments. For example, affinity matured anti-CGRP antagonist antibody can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896; and WO2004/058184).

The following methods may be used for adjusting the affinity of an antibody, anti-CGRP antagonist antibody, and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased or no binding are identified. Methods for determining binding affinity are well-known in the art. Binding affinity may be determined using Biacore surface plasmon resonance analysis, which detects differences in binding affinity of about 2-fold or greater. Biacore is particularly useful when the starting antibody already binds with a relatively high affinity, for example a K_{D} of about 10 nM or lower. Screening using Biacore surface plasmon resonance is described in the Examples, herein.

Binding affinity may be determined using Kinexa Biocensor, scintillation proximity assays, ELISA, ORIGEN immunoassay (IGEN), fluorescence quenching, fluorescence transfer, and/or yeast display. Binding affinity may also be screened using a suitable bioassay.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened includes substitutions in two or more positions, which may be in the same CDR or in two or more CDRs. Thus, the library may comprise substitutions in two or more positions in one CDR. The library may include substitution in two or more positions in two or more CDRs. The library may comprise substitutions in 3, 4, 5, or more positions, said positions found in two, three, four, five or six CDRs. The substitution may be prepared using low redundancy codons. See, e.g., Table 2 of Balint et al. (1993) Gene 137(1):109-18).

The CDR may be CDRH3 and/or CDRL3. The CDR may be one or more of CDRL1, CDRL2, CDRL3, CDRH1, CDRH2, and/or CDRH3. The CDR may be a Kabat CDR, a Chothia CDR, or an extended CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also include substitution(s) at positions that did not show improved affinity in the first round of screening.

The second library is screened or selected for library members with improved and/or altered binding affinity using any method known in the art, including screening using Biacore surface plasmon resonance analysis, and selection using any method known in the art for selection, including phage display, yeast display, and ribosome display.

The disclosure also encompasses fusion proteins including one or more fragments or regions from the antibodies (such as G1) or polypeptides of this disclosure. In one embodiment, a fusion polypeptide is provided that includes at least 10 contiguous amino acids of the variable light chain region shown in SEQ ID NO:2 (FIG. 5) and/or at least 10 amino acids of the variable heavy chain region shown in SEQ ID NO:1 (FIG. 5). In other embodiments, a fusion polypeptide is provided that includes at least about 10, at least about 15, at least about 20, at least about 25, or at least about 30 contiguous amino acids of the variable light chain region shown in SEQ ID NO:2 (FIG. 5) and/or at least about 10, at least about 15, at least about 20, at least about 25, or at least about 30 contiguous amino acids of the variable heavy chain region shown in SEQ ID NO:1 (FIG. 5). In another embodiment, the fusion polypeptide includes a light chain variable region and/or a heavy chain variable region of G1, as shown in SEQ ID NO:2 and SEQ ID NO:1 of FIG. 5. In another embodiment, the fusion polypeptide includes one or more CDR(s) of G1. In still other embodiments, the fusion polypeptide comprises CDR H3 and/or CDR L3 of antibody G1. For purposes of this disclosure, a G1 fusion protein contains one or more G1 antibodies and another amino acid sequence to which it is not attached in the native molecule, for example, a heterologous sequence or a homologous sequence from another region. Exemplary heterologous sequences include, but are not limited to a “tag” such as a FLAG tag or a 6His tag (SEQ ID NO: 56). Tags are well known in the art.

A G1 fusion polypeptide can be created by methods known in the art, for example, synthetically or recombinantly. Typically, the G1 fusion proteins of this disclosure are made by preparing an expressing a polynucleotide encoding them using recombinant methods described herein, although they may also be prepared by other means known in the art, including, for example, chemical synthesis.

This disclosure also provides compositions comprising antibodies or polypeptides derived from G1 conjugated (for example, linked) to an agent that facilitates coupling to a solid support (such as biotin or avidin). For simplicity, reference will be made generally to G1 or antibodies with the understanding that these methods apply to any of the CGRP binding embodiments described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

An antibody or polypeptide of this disclosure may be linked to a labeling agent (alternatively termed “label”) such as a fluorescent molecule, a radioactive molecule, an enzyme, a hapten, a chemiluminescent molecule, or any other label known in the art. Labels are known in the art which generally provide (either directly or indirectly) a signal.

The disclosure also provides compositions (including pharmaceutical compositions) and kits that include antibody G1, and, any one or more of (or all of) the antibodies (e.g., anti-CGRP antagonist antibodies) and/or polypeptides described herein.

The disclosure also provides isolated polynucleotides encoding the antibodies and polypeptides (including an antibody comprising the polypeptide sequences of the light chain and heavy chain variable regions shown in FIG. 5), and vectors and host cells comprising the polynucleotide.

Accordingly, the disclosure provides polynucleotides (or compositions, including pharmaceutical compositions), including polynucleotides encoding any of the following: (a) antibody G1 or its variants shown in Table 6; (b) a fragment or a region of antibody G1 or its variants shown in Table 6; (c) a light chain of antibody G1 or its variants shown in Table 6; (d) a heavy chain of antibody G1 or its variants shown in Table 6; (e) one or more variable region(s) from a light chain and/or a heavy chain of antibody G1 or its variants shown in Table 6; (f) one or more CDR(s) (one, two, three, four, five or six CDRs) of antibody G1 or its variants shown in Table 6; (g) CDR H3 from the heavy chain of antibody G1; (h) CDR L3 from the light chain of antibody G1 or its variants shown in Table 6; (i) three CDRs from the light chain of antibody G1 or its variants shown in Table 6; (j) three CDRs from the heavy chain of antibody G1 or its variants shown in Table 6; (k) three CDRs from the light chain and three CDRs from the heavy chain, of antibody G1 or its variants shown in Table 6; and (1) an antibody comprising any one of (b) through (k). In some embodiments, the polynucleotide includes either or both of the polynucleotide(s) shown in SEQ ID NO: 9 and SEQ ID NO: 10.

In one aspect, the disclosure provides polynucleotides encoding any of the antibodies (including antibody fragments) and polypeptides described herein, such as antibodies and polypeptides having impaired effector function. Polynucleotides can be made by procedures known in the art.

In one aspect, the disclosure provides compositions (such as a pharmaceutical compositions) comprising any of the polynucleotides of the disclosure. In some embodiments, the composition includes an expression vector comprising a polynucleotide encoding the G1 antibody as described herein. In some embodiment, the composition includes an expression vector comprising a polynucleotide encoding any of the antibodies or polypeptides described herein. In still other embodiments, the composition includes either or both of the polynucleotides shown in SEQ ID NO:9 and SEQ ID NO:10. Expression vectors, and administration of polynucleotide compositions are further described herein.

In one aspect, the disclosure provides a method of making any of the polynucleotides described herein.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials.

Polynucleotides may include a native sequence (i.e., an endogenous sequence that encodes an antibody or a portion thereof) or may include a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants can exhibit at least about 70% identity, at least about 80% identity, at least about 90% identity, at least 95% identity or at least 99% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

In one example, the “percentage of sequence identity” is determined by comparing two optimally aligned sequences over a window of comparison of at least 20 positions, wherein the portion of the polynucleotide or polypeptide sequence in the comparison window may comprise additions or deletions (i.e, gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. Thus, in some examples, any of the antibodies or antibody fragments provided herein that can be used with the disclosed methods can have at least 90%, at least 95%, at least 96%, at least 97%, at least 98%, at least 99% sequence identity to the antibodies or antibody fragments provided herein (e.g., have such sequence identity to any of SEQ ID NOS: 1-14 or 155-174)

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells can be transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston (1994).

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., (1989), for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The disclosure also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). In some examples, the host cells express the cDNAs at a level of about at least 5 fold higher, at least 10 fold higher, or at least 20 fold higher than that of the corresponding endogenous antibody or protein of interest, if present, in the host cells. Screening the host cells for a specific binding to CGRP effected by an immunoassay or FACS. A cell overexpressing the antibody or protein of interest can be identified.

### Pharmaceutical Compositions

A composition including one or more anti-CGRP antagonist antibodies can be formulated to provide an effective amount of one or more anti-CGRP antagonist antibodies as the active ingredients, or a pharmaceutically acceptable salt, ester, prodrug, solvate, hydrate or derivative thereof. The composition can be a pharmaceutical composition. The composition can further include one or more additional therapeutic agents that are selected for their particular usefulness against a condition that is being treated. For example, the therapeutic agent may be an anti-diabetes agent. The anti-diabetes agent can be in an amount effective in treating a symptom associated with diabetes. Where desired, the compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

In some embodiments, the compositions used in the methods of the disclosure include an effective amount of one ormore anti-CGRP antagonist antibodies or an anti-CGRP antagonist antibody derived polypeptide described herein. In one embodiment, the composition further includes one or more additional CGRP antagonists. In some embodiments, the anti-CGRP antagonist antibody binds human CGRP. In some embodiments, the anti-CGRP antagonist antibody is humanized. In some embodiment, the anti-CGRP antagonist antibody comprises a constant region that does not trigger an unwanted or undesirable immune response, such as antibody-mediated lysis or ADCC. In some embodiments, the anti-CGRP antagonist antibody includes one or more CDR(s) of antibody G1 (such as one, two, three, four, five, or, in some embodiments, all six CDRs from G1). In some embodiments, the anti-CGRP antagonist antibody is a human antibody.

The compositions can include more than one anti-CGRP antagonist antibody (e.g., a mixture of anti-CGRP antagonist antibodies that recognize different epitopes of CGRP). Other exemplary compositions include more than one anti-CGRP antagonist antibodies that recognize the same epitope(s), or different species of anti-CGRP antagonist antibodies that bind to different epitopes of CGRP. A composition can include polyclonal anti-CGRP antagonist antibodies. A composition can include a mixture of two or more monoclonal anti-CGRP antagonist antibodies (e.g., 2, 3, 4, 5, or more monoclonal antibodies).

The composition used in the disclosed methods can further include pharmaceutically acceptable carriers, excipients, or stabilizers (Remington: The Science and practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and can comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENTm, PLURONICS™ or polyethylene glycol (PEG). Pharmaceutically acceptable excipients are further described herein.

Such a composition can be prophylactically and/or therapeutically suitable or beneficial. The composition can be suitable for relatively rapid anti-CGRP antagonist antibody intake, provision, and/or supplementation, as can be suitable or beneficial for any of a variety of applications, such as a nutritional or prophylactic application, and/or a therapeutic application. The composition can be a suitable or beneficial vehicle for anti-CGRP antagonist antibody intake, provision, and/or supplementation application(s), such as any that may be accomplished via a dietary vehicle or a consumable vehicle, such as a foodstuff and/or a beverage, for example.

In some embodiments, the composition including the one or more anti-CGRP antagonist antibodies can be in a single dosage form. In some cases, the composition can be in multiple dosage forms. The compositions can be administered in a single dose or multiple doses. Dosing may be about once, twice, three times, four times, five times, six times, or more than six times per day. Dosing may be about once a month, once every two weeks, once a week, or once every other day. In some embodiments, dosing may be at least once daily. In another embodiment, the one or more anti-CGRP antagonist antibodies and/or one or more additional therapeutic agents (e.g., anti-metabolic disorder agents or anti-diabetes agents) can be administered together about once per day to about 6 times per day.

In another embodiment, the administration of anti-CGRP antagonist antibody and/or one or more additional therapeutic agents may continue for less than about 7 days. In yet another embodiment the administration continues for equal or more than about 6, 10, 14, 20, 28, 30 days, 2, 3, 4, 5, 6, 7, 8, 9, 10, 11, 12 months, 2, 3, 4, 5, 6, 7, 8, 9, 10, 11, 12, 13, 14, 15, 16, 17, 18, 19, 20, 21, 22, 23, 24, 25, 26, 27, 28, 29, 30 years. In some cases, continuous dosing is achieved and maintained as long as necessary. In some embodiments, the composition including one or more anti-CGRP antagonist antibodies is administered for at least about a month. In some embodiments, the composition is administered for at least about 10 days. Administration of the composition comprising anti-CGRP antagonist antibody may continue as long as necessary. In some embodiments, a compound of the disclosure is administered for less than 28, 14, 7, 6, 5, 4, 3, 2, or 1 day. In some embodiments, a compound of the disclosure is administered chronically on an ongoing basis, e.g., for the treatment of chronic effects.

The compositions of the disclosure may be administered in dosages. It is known in the art that due to inter-subject variability in compound pharmacokinetics, individualization of dosing regimen may be desired for optimal therapy. Dosing for a compound of the disclosure may be found by routine experimentation in light of the instant disclosure.

Determining an appropriate dosage for administration of a composition comprising one or more anti-CGRP antagonist antibodies to a subject may take into account any of a variety of factors, such as those just mentioned, for example, any potential or actual side-effect(s), and/or a purpose of the administration of the composition comprising anti-CGRP antagonist antibody, such as a condition treatment purpose, a reducing side-effect purpose, and/or other purpose(s) for which the composition comprising anti-CGRP antagonist antibody may be administered to a subject. Determining an appropriate dosage may take into account any of these factors, any other suitable factor(s), any side-effect(s), animal study modeling, human study modeling, clinical study modeling, drug study modeling, and any balancing therebetween.

The amount of anti-CGRP antagonist antibody that can be absorbed by a subject, or the rate of absorption of anti-CGRP antagonist antibody by a subject may vary from subject to subject, based on any of a variety of factors. Examples of such factors include metabolic rate, kidney function, overall health, and/or other factor(s) concerning a subject, and a property or nature of the composition comprising the anti-CGRP antagonist antibody itself, such as the counter ion, any enhancing agent, its administration vehicle or method, and/or other factor(s) concerning the composition including one or more anti-CGRP antagonist antibodies and/or its administration to a subject.

In some embodiments, a formulation comprising one or more anti-CGRP antagonist antibodies can be prepared for any suitable route of administration. In some examples, the one or more anti-CGRP antagonist antibodies are present in an amount ranging from 0.1 mg to 3000 mg, 1 mg to 1000 mg, 100 to 1000 mg, 100 to 500 mg, 10 mg to 100 mg, or 10 mg to 25 mg. A formulation comprising one or more anti-CGRP antagonist antibodies can be prepared for any suitable route of administration with an antibody amount of, at most, or at least 0.1 mg, 1 mg, 100 mg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or 3000 mg.

In some embodiments, a liquid formulation comprising one or more anti-CGRP antagonist antibodies can be prepared for any suitable route of administration with an antibody concentration ranging from 0.1 to 500 mg/mL, 0.1 to 375 mg/mL, 0.1 to 250 mg/mL, 0.1 to 175 mg/mL, or 0.1 to 100 mg/mL. In some embodiments, a liquid formulation comprising one or more anti-CGRP antagonist antibodies may be prepared for any suitable route of administration with an antibody concentration of, of at most, of at least, or less than 0.1, 0.5, 1, 5, 10, 15 20, 25, 30, 35, 40, 45, 50, 55, 60, 65, 70, 75, 80, 85, 90, 95, 100, 105 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/mL.

In some embodiments, an initial dose (e.g., a loading dose) of an anti-CGRP antagonist antibody may be administered to a subject, followed by administration of one or more additional doses at desired intervals. In some embodiments, the initial dose and one or more of the additional doses are the same dose. In some embodiments, the one or more additional doses are a different dose than the initial dose. In some embodiments, the frequency at which the one or more additional doses are administered is constant (e.g., every month). In some embodiments, the frequency at which the one or more additional doses are administered is variable (e.g., one additional dose administered at one month following the initial dose, followed by another additional dose at three months following the initial dose). Any desirable and/or therapeutic regimen of initial loading dose, additional doses, and frequency (e.g., including those described herein) of additional doses may be used. An exemplary regimen includes an initial loading dose of 675 mg anti-CGRP antagonist antibody administered subcutaneously, followed by subsequent maintenance doses of 225 mg of the antibody administered subcutaneously at one month intervals.

In some embodiments, an initial dose of one or more anti-CGRP antagonist antibodies of 0.1 μg, 1 μg, 100 μg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or 3000 mg may be administered to a subject followed by one or more additional doses of one or more anti-CGRP antagonist antibodies of 0.1 μg, 1 μg, 100 μg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or 3000 mg.

In some embodiments, a dose of anti-CGRP antagonist antibody(s) may be divided into sub-doses and administered as multiple sub-doses, depending, for example, on the route of administration and/or particular formulation administered. For example, in cases where a dose is administered subcutaneously, the subcutaneous dose may be divided into multiple sub-doses and each sub-dose administered at a different site in order to avoid, for example, a larger, single subcutaneous injection at a single site. For example, a subcutaneous dose of 900 mg may be divided into four sub-doses of 225 mg each and each 225 mg dose administered at a different site, which can help minimize the volume injected at each site. The division of sub-doses may be equal (e.g., 4 equal sub-doses) or may be unequal (e.g., 4 sub-doses, two of the sub-doses twice as large as the other sub-doses).

In some embodiments, the number of doses of anti-CGRP antagonist antibody(s) administered to a subject over the course of treatment may vary depending upon, for example, achieving reduced incidence of a metabolic disorder symptom in the subject. For example, the number of doses administered over the course of treatment may be, may be at least, or may be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some cases, treatment may be given indefinitely. In some cases, treatment may be acute such that at most 1, 2, 3, 4, 5, or 6 doses are administered to a subject for treatment.

In some embodiments, a dose (or sub-dose) of one or more anti-CGRP antagonist antibodies can be formulated in a liquid formulation and administered (e.g., via subcutaneous injection) to a subject as a liquid. In such cases, the volume of liquid formulation including one or more anti-CGRP antagonist antibodies administered to the subject may vary depending upon, for example, the concentration of anti-CGRP antagonist antibody in the liquid formulation, the desired dose of one or more anti-CGRP antagonist antibodies, and/or the route of administration used. For example the volume of liquid formulation including one or more anti-CGRP antagonist antibodies and administered (e.g., via an injection, such as, for example, a subcutaneous injection) to a subject may be from 0.001 mL to 10.0 mL, 0.01 mL to 5.0 mL, 0.1 mL to 5 mL, 0.1 mL to 3 mL, 0.5 mL to 2.5 mL, or 1 mL to 2.5 mL. For example, the volume of liquid formulation including one or more anti-CGRP antagonist antibodies and administered (e.g., via an injection, such as, for example, a subcutaneous injection) to a subject may be, may be at least, may be less than, or may be at most 0.001, 0.005, 0.01, 0.02, 0.03, 0.04, 0.05, 0.06, 0.07, 0.08, 0.09, 0.10, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, or 10.0 mL.

An anti-CGRP antagonist antibody can be administered using any suitable method, including by injection (e.g., intraperitoneally, intravenously, subcutaneously, intramuscularly, etc.). Anti-CGRP antagonist antibodies can also be administered via inhalation, as described herein. Generally, for administration of anti-CGRP antagonist antibodies, an initial candidate dosage can be about 2 mg/kg. For the purpose of the present disclosure, a typical daily dosage might range from about any of 3 μg/kg to 30 μg/kg to 300 μg/kg to 3 mg/kg, to 30 mg/kg to 100 mg/kg or more, depending on the factors mentioned above. For example, dosage of about 1 mg/kg, about 2.5 mg/kg, about 5 mg/kg, about 10 mg/kg, and about 25 mg/kg may be used. For repeated administrations over several days or longer, depending on the condition, the treatment is sustained until a desired suppression of symptoms occurs or until sufficient therapeutic levels are achieved, for example, to reduce pain. An exemplary dosing regimen includes administering an initial dose of about 2 mg/kg, followed by a weekly maintenance dose of about 1 mg/kg of the anti-CGRP antagonist antibody, or followed by a maintenance dose of about 1 mg/kg every other week. Another exemplary dosing regimen includes administering a dose of 100 mg, 125 mg, 150 mg, 200 mg, 225 mg, 250 mg, 275 mg, 300 mg, 350 mg, 400 mg, 450 mg, 500 mg, 550 mg, 600 mg, 675 mg, or 900 mg to a subject once per month subcutaneously. Another exemplary dosing regimen includes administering an initial dose of 675 mg subcutaneously, followed by a monthly dose of 225 mg of the antibody subcutaneously. However, other dosage regimens may be useful, depending on the pattern of pharmacokinetic decay that the practitioner wishes to achieve. For example, in some embodiments, dosing from one-four times a week is contemplated. The progress of this therapy is easily monitored by conventional techniques and assays. The dosing regimen (including the CGRP antagonist(s) used) can vary over time.

In some embodiments, the dose of an anti-CGRP antagonist antibody may range from 0.1 μg to 3000 mg, 1 mg to 1000 mg, 100 to 1000 mg, or 100 to 500 mg. In some embodiments, the dose of an anti-CGRP antagonist antibody may be, may be at most, may be less than, or may be at least 0.1 μg, 1 μg, 100 μg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or 3000 mg.

In some embodiments, the dose of an anti-CGRP antagonist antibody may range from 0.1 to 500, 0.1 to 100, 0.1 to 50, 0.1 to 20, or 0.1 to 10 mg/kg of body weight. In some embodiments, the dose of an anti-CGRP antagonist antibody may be, may be at most, may be less than, or may be at least 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or 500 mg/kg of body weight.

Empirical considerations, such as the half-life, can contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of headache (e.g., metabolic disorder). Alternatively, sustained continuous release formulations of anti-CGRP antagonist antibodies may be appropriate. Various formulations and devices for achieving sustained release are known in the art.

In one embodiment, dosages for an anti-CGRP antagonist antibody may be determined empirically in individuals who have been given one or more administration(s) of an anti-CGRP antagonist antibody. Individuals are given incremental dosages of an anti-CGRP antagonist antibody. To assess efficacy of an anti-CGRP antagonist antibody, an indicator of the disease can be followed.

Administration of one or more anti-CGRP antagonist antibodies in accordance with the method in the present disclosure can be continuous or intermittent, depending, for example, upon the recipient's physiological condition, whether the purpose of the administration is therapeutic or prophylactic, and other factors known to skilled practitioners. The administration of an anti-CGRP antagonist antibody may be essentially continuous over a preselected period of time or may be in a series of spaced dose, e.g., either before, during, or after developing a metabolic disorder (e.g., diabetes); before; during; before and after; during and after; before and during; or before, during, and after developing a metabolic disorder. Administration can be before, during and/or after any event likely to give rise to a metabolic disorder.

In some embodiments, more than one anti-CGRP antagonist antibody may be present. At least one, at least two, at least three, at least four, at least five different, or more anti-CGRP antagonist antibodies can be present. Generally, those anti-CGRP antagonist antibodies may have complementary activities that do not adversely affect each other. Non-limiting examples of the anti-CGRP antagonist antibodies can include ALD403 (an anti-CGRP antibody from Alder BioPharmaceuticals), LY2951742 (an anti-CGRP antibody from Arteaus Therapeutics), or those described in PCT Patent Application No. WO2012162243, U.S. Pat. No. 8,450,327, PCT Patent Application No. WO2010075238, and European Patent Application No. EP0348490, all hereby incorporated by reference in their entirety. An anti-CGRP antagonist antibody can also be used in conjunction with other CGRP antagonists. For example, one or more of the following CGRP antagonists may be used: an anti-sense molecule directed to a CGRP (including an anti-sense molecule directed to a nucleic acid encoding CGRP), a CGRP inhibitory compound, a CGRP structural analog, and a dominant-negative mutation of a CGRP receptor that binds a CGRP. An anti-CGRP antagonist antibody can also be used in conjunction with other agents that serve to enhance and/or complement the effectiveness of the agents.

As mentioned above, such a dosage may be determined, modified and/or refined based on any suitable factor(s), such as results of clinical trials concerning subjects, for example human subjects. In some embodiments, a suitable dosage may be determined, modified and/or refined based on a determination of a suitable dosage for a suitable animal model, based on experimental studies or tests, for example, and conversion of such a suitable animal dosage to a suitable human dosage, based on suitable conversion factor(s), such as any suitable established conversion factor(s), for example. Further by way of example, it is contemplated that any such suitable human dosage may be further determined, modified and/or refined based on clinical trials involving human subjects, for example.

A composition of the disclosure can include an active ingredient (e.g., anti-CGRP antagonist antibody) of the present disclosure or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizer and adjuvants. The composition can further include one or more supplements or other active ingredients. The composition can further include one or more coloring pigments.

Described below are non-limiting exemplary compositions and methods for preparing the same. In some embodiments, the disclosure provides a pharmaceutical composition including one or more anti-CGRP antagonist antibodies, and a pharmaceutical excipient. A composition including one or more anti-CGRP antagonist antibodies appropriate for administration to a subject may be provided in any suitable form, such as a liquid form, a gel form, a semi-liquid (for example, a liquid, such as a viscous liquid, containing some solid) form, a semi-solid (a solid containing some liquid) form, and/or a solid form, for example. Merely by way of example, a tablet form, a capsule form, a food form a chewable form, a non-chewable form, a slow- or sustained-release form, a non-slow- or non-sustained-release from, and/or the like, may be employed. Gradual-release tablets are known in the art. Examples of such tablets are set forth in U.S. Pat. No. 3,456,049. Such a composition may include an additional agent or agents, whether active or passive. Examples of such an agent includes but is not limited a sweetening agent, a flavoring agent, a coloring agent, a filling agent, a binding agent, a lubricating agent, an excipient, a preservative, a manufacturing agent, and/or the like, merely by way of example, in any suitable form. A slow- or sustained-release form may delay disintegration and/or absorption of the composition and/or one or more component(s) thereof over a period, such as a relatively long period, for example. A food form may take the form of a food bar, a cereal product, a bakery product, a dairy product, and/or the like, for example. A bakery product form may take the form of a bread-type product, such as a bagel or bread itself, for example, a donut, a muffin, and/or the like, merely by way of example. A component of a composition including one or more anti-CGRP antagonist antibodies can be provided in a form that is other than that of another component of the composition. For example, anti-CGRP antagonist antibody may be provided in a solid form, such as solid food or cereal that is taken with an anti-metabolic disorder agent in a liquid form. Such administration of compositions including one or more anti-CGRP antagonist antibodies in multiple forms, may occur simultaneously, or at different times.

In some embodiments, the composition including one or more anti-CGRP antagonist antibodies is a solid or liquid pharmaceutical composition suitable for injection. Pharmaceutical compositions of the disclosure suitable for injection can be presented as a discrete or unit dosage form each containing a predetermined amount of an active ingredient. The predetermined amount of the active ingredients may be a powder or granules, beads, a solution, or a suspension in an aqueous or non-aqueous liquid, an oil-in-water emulsion, or a water-in-oil liquid emulsion. The subject dosage forms can be prepared by any of the methods of pharmacy. In some cases, the methods may include the step of bringing the active ingredient into association with the carrier, which constitutes one or more desired ingredients. The compositions may be prepared by uniformly and intimately admixing the active ingredient with liquid carriers or finely divided solid carriers or both, and then, if desired, shaping the product into the desired presentation.

This disclosure further encompasses anhydrous pharmaceutical compositions and dosage forms comprising an active ingredient (such as one or more anti-CGRP antagonist antibodies), since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient (such as one or more anti-CGRP antagonist antibodies) can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, including but not limited to, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, granules and tablets, with the solid oral preparations. If desired, tablets can be coated by standard aqueous or nonaqueous techniques.

Binders suitable for use in the subject pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, talcum, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, povidone, povidone k30, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When the compositions including one or more anti-CGRP antagonist antibodies are desired for oral administration, the composition therein may be combined with various sweetening or flavoring agents (e.g., aspartame, sugar), coloring matter or dyes (e.g., yellow pigment) and, if so desired, emulsifying and/or suspending agents, together with such diluents as water, ethanol, propylene glycol, glycerin and various combinations thereof.

Sweeteners, sweetening agents or flavoring agents are substances that sweeten food, beverages, or pharmaceutical compositions. Sweeteners can be sugar, saccharine or other low-calorie synthetic products (From Random House Unabridged Dictionary, 2d ed). Non-limiting examples of the sweetening or flavoring agent include N—(N-(3-(3-hydroxy-4-methoxyphenyl) propyl)-alpha-aspartyl)-L-phenylalanine 1-methyl ester (Aspartame), 11-oxo-mogroside V, aspartyl-alanine fenchyl ester, abrusoside A methyl ester, neotame, osladin, SC 45647, cellobiofructose, gentiobiofructose, 4,4′,6,6′-tetrachloro-4,4′,6,6′-tetradeoxygalactotrehalose, brazzein protein, Pentadiplandra brazzeana, hydrangenol-4′-O-glucoside, hydrangenol-8-O-galactoside, 3,3′-dideoxytrehalose, selligueain A, periandrin V, NC 174, mabinlin protein, Capparis masaikai, alitame, cyclocarioside A, mabilin II protein, Capparis masaikai, N-(4-cyanophenyl)-N′-(2-carboxyethyl)urea, steviolbio side, Adentol, periandradulcin C, periandradulcin B, periandradulcin A, Sweetrex, monellin, Asn(22)-Gln(25)-Asn(26)-A-chain-Asn(49)-Glu(50)-B-chain, curculin, leucrose, polypodoside A, rubusoside, glycyrrhetyl 3-monoglucuronide, oxime V, hernandulcin, fungitetraose, neosugar, mogroside IV, mogroside V, mogroside VI, coupling sugar, 4-chlorokynurenine, glucosylsucrose, 6-chlorotryptophan, rebaudioside A, perillartine, hydrangenol, Palatinit, Lycasin, cycloheptylsulfamate, CH 401-Na, neohesperidin dihydrochalcone, P 4000, maltitol, phyllodulcin, miraculin protein, Synsepalum dulcificum, acetosulfam or dulcin.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition can include a solubilizer to ensure good solubilization and/or dissolution of the compound of the present disclosure and to minimize precipitation of the compound of the present disclosure. This can be especially beneficial for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, citrate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Other exemplary solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids that can be used include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Aqueous solutions in saline are also conventionally used for injection. Examples include ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. The compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Liposomes containing one or more anti-CGRP antagonist antibodies can be prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

### Kits

The disclosure also provides kits for use in the instant methods. Kits of the disclosure include one or more containers comprising one or more anti-CGRP antagonist antibodies (such as a humanized antibody) or polypeptide described herein and instructions for use in accordance with any of the methods of the disclosure described herein. Generally, these instructions include a description of administration of the one or more anti-CGRP antagonist antibodies to treat, ameliorate or prevent a metabolic disorder or a condition associated with a metabolic disorder (such as diabetes) according to any of the methods described herein. The kit may further include a description of selecting an individual suitable for treatment based on identifying whether that individual has a metabolic disorder or whether the individual is at risk of having a metabolic disorder. In still other embodiments, the instructions include a description of administering an anti-CGRP antagonist antibody to an individual at risk of having a metabolic disorder (such as diabetes).

In some embodiments, the antibody is a humanized antibody. In some embodiments, the antibody is human. In some embodiments, the antibody is a monoclonal antibody. In some embodiments. In some embodiments, the antibody includes one or more CDR(s) of antibody G1 (such as one, two, three, four, five, or, in some embodiments, all six CDRs from G1).

The instructions relating to the use of an anti-CGRP antagonist antibody typically include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

In some embodiments, the label or package insert indicates that the composition is used for treating, ameliorating and/or preventing a metabolic disorder (such as diabetes). Instructions may be provided for practicing any of the methods described herein.

In some examples, the kits include one or more additional therapeutic agents, such as one or more anti-metabolic disorder agents or anti-diabetes agents known in the art or provided herein. Thus, the kit can include one or more of such as 1, 2, 3, 4, or 5 of) HMG-CoA inhibitors (or statins), biguanides, sufonylureas, thiazolidinediones, meglitinides glinides, α-glucosidase inhibitors, repaglinide, nateglinide, DPP-IV inhibitor, sitagliptin, vildagliptin, saxagliptin or insulin and insulin analogues. Other exemplary additional anti-metabolic disorder agents or anti-diabetes agents are provided above. Such additional therapeutic agents can be in the same, or separate containers as the anti-CGRP antagonist antibody.

The kits of this disclosure are typically in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). In some embodiments, at least one active agent in the composition is an anti-CGRP antagonist antibody. The container may further include a second pharmaceutically active agent or a therapeutic agent.

Kits may optionally provide additional components such as buffers and interpretive information. Normally, the kit comprises a container and a label or package insert(s) on or associated with the container.

### Example 1

### Generation and Characterization of Monoclonal Antibodies Directed Against CGRP

Generation of anti-CGRP antibodies. To generate anti-CGRP antibodies that have cross-species reactivity for rat and human CGRP, mice were immunized with 25-100 μg of human α-CGRP or β-CGRP conjugated to KLH in adjuvant (50 μl per footpad, 100 μl total per mouse) at various intervals. Immunization was generally performed as described in Geerligs et al., 1989, J. Immunol. Methods 124:95-102; Kenney et al., 1989, J. Immunol. Methods 121:157-166; and Wicher et al., 1989, Int. Arch. Allergy Appl. Immunol. 89:128-135. Mice were first immunized with 50 μg of human α-CGRP or β-CGRP conjugated to KLH in CFA (complete Freund's adjuvant). After 21 days, mice were secondly immunized with 25 μg of human β-CGRP (for mice first immunized with human α-CGRP) or α-CGRP (for mice first immunized with human β-CGRP) conjugated to KLH in IFA (incomplete Freund's adjuvant). Twenty three days later after the second immunization, third immunization was performed with 25 μg of rat α-CGRP conjugated to KLH in IFA. Ten days later, antibody titers were tested using ELISA. Forth immunization was performed with 25 μg of the peptide (rat α-CGRP-KLH) in IFA 34 days after the third immunization. Final booster was performed with 100 μg soluble peptide (rat α-CGRP) 32 days after the forth immunization.

Splenocytes were obtained from the immunized mouse and fused with NSO myeloma cells at a ratio of 10:1, with polyethylene glycol 1500. The hybrids were plated out into 96-well plates in DMEM containing 20% horse serum and 2-oxaloacetate/pyruvate/insulin (Sigma), and hypoxanthine/aminopterin/thymidine selection was begun. On day 8, 100 μl of DMEM containing 20% horse serum was added to all the wells. Supernatants of the hybrids were screened by using antibody capture immunoassay. Determination of antibody class was done with class-specific second antibodies.

A panel of monoclonal antibody-producing cell lines was selected based on their binding to human and rat CGRP for further characterization. These antibodies and characteristics are shown below in Tables 2 and 3.

Purification and Fab fragment preparation. Monoclonal antibodies selected for further characterization were purified from supernatants of hybridoma cultures using protein A affinity chromatography. The supernatants were equilibrated to pH 8. The supernatants were then loaded to the protein A column MabSelect (Amersham Biosciences #17-5199-02) equilibrated with PBS to pH 8. The column was washed with 5 column volumes of PBS, pH 8. The antibodies were eluted with 50 mM citrate-phosphate buffer, pH 3. The eluted antibodies were neutralized with 1M Phosphate Buffer, pH 8. The purified antibodies were dialyzed with PBS, pH 7.4. The antibody concentrations were determined by SDS-PAGE, using a murine monoclonal antibody standard curve.

Fabs were prepared by papain proteolysis of the full antibodies using Immunopure Fab kit (Pierce #44885) and purified by flow through protein A chromatography following manufacturer instructions. Concentrations were determined by ELISA and/or SDS-PAGE electrophoresis using a standard Fab of known concentration (determined by amino acid analysis), and by A280 using 10D=0.6 mg/ml (or theoretical equivalent based on the amino acid sequence).

Affinity determination of the Fabs. Affinities of the anti-CGRP monoclonal antibodies were determined at either 25° C. or 37° C. using the Biacore3000™ surface plasmon resonance (SPR) system (Biacore, INC, Piscataway N.J.) with the manufacture's own running buffer, HBS-EP (10 mM HEPES pH 7.4, 150 mM NaCl, 3 mM EDTA, 0.005% v/v polysorbate P20). Affinity was determined by capturing N-terminally biotinylated CGRP peptides (custom ordered from GenScript Corporation, New Jersey or Global Peptide Services, Colorado) via pre-immobilized streptavidin on SA chip and measuring binding kinetics of antibody Fab titrated across the CGRP surface. Biotinylated CGRP was diluted into HBS-EP and injected over the chip at a concentration of less than 0.001 mg/ml. Using variable flow time across the individual chip channels, two ranges of antigen density were achieved: <50 response units (RU) for detailed kinetic studies and about 800 RU for concentration studies and screening. Two- or three-fold serial dilutions typically at concentrations spanning 1 μM-0.1 nM (aimed at 0.1-10× estimated K_{D}) of purified Fab fragments were injected for 1 minute at 100 μL/min and dissociation times of 10 minutes were allowed. After each binding cycle, surfaces were regenerated with 25 mM NaOH in 25% v/v ethanol, which was tolerated over hundreds of cycles. Kinetic association rate (kₒₙ) and dissociation rate (k_{off}) were obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Global equilibrium dissociation constants (K_{D}) or “affinities” were calculated from the ratio K_{D}=k_{off}/kₒₙ. Affinities of the murine Fab fragments are shown in Tables 2 and 3.

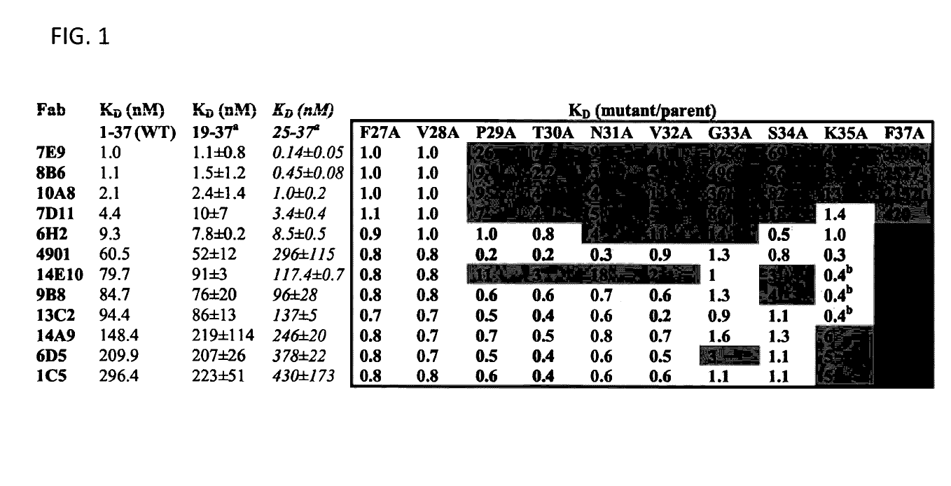

Epitope mapping of the murine anti-CGRP antibodies. To determine the epitope that anti-CGRP antibodies bind on human α-CGRP, binding affinities of the Fab fragments to various CGRP fragments were measured as described above by capturing N-terminally biotinylated CGRP fragments amino acids 19-37 and amino acids 25-37 on a SA sensor chip. FIG. 1 shows their binding affinities measured at 25° C. As shown in FIG. 1, all antibodies, except antibody 4901, bind to human α-CGRP fragments 19-37 and 25-37 with affinity similar to their binding affinity to full length human α-CGRP (1-37). Antibody 4901 binds to human α-CGRP fragment 25-37 with six fold lower affinity than binding to full length human α-CGRP fragment, due mainly to a loss in off-rate. The data indicate that these anti-CGRP antibodies generally bind to the C-terminal end of CGRP.

Alanine scanning was performed to further characterize amino acids in human α-CGRP involved in binding of anti-CGRP antibodies. Different variants of human α-CGRP with single alanine substitutions were generated by peptide synthesis. Their amino acid sequences are shown in Table 4 along with all the other peptides used in the Biacore analysis. Affinities of Fab fragments of the anti-CGRP antibodies to these variants were determined using Biacore as described above. As shown in FIG. 1, all 12 antibodies target a C-terminal epitope, with amino acid F37 being the most crucial residue. Mutation of F37 to alanine significantly lowered the affinity or even completely knocked out binding of the anti-CGRP antibodies to the peptide. The next most important amino acid residue is G33, however, only the high affinity antibodies (7E9, 8B6, 10A8, and 7D11) were affected by alanine replacement at this position. Amino acid residue S34 also plays a significant, but lesser, role in the binding of these four high affinity antibodies.

TABLE 2
Characteristics of the anti-CGRP monoclonal antibodies'
binding to human α-CGRP and their antagonist activity

| | | | Cell-based blocking human | IC₅₀ (nM binding sites) |
|---|---|---|---|---|
| | K_{D} to human | K_{D} to human | α-CGRP binding to its | at 25° C. (room temp.) |
| | α-CGRP at | α-CGRP at | receptor at 25° C. (measured | measured in radioligand |
| Antibodies | 25° C. (nM) | 37° C. (nM) | by cAMP activation) | binding assay. |
| 7E9 | 1.0 | 0.9 | Yes | 2.5 |
| 8B6 | 1.1 | 1.2 | Yes | 4.0 |
| 10A8 | 2.1 | 3.0 | Yes | n.d. |
| 7D11 | 4.4 | 5.4 | Yes | n.d. |
| 6H2 | 9.3 | 42 | Yes | 12.9 |
| 4901 | 61 | 139 | Yes | 58 |
| 14E10 | 80 | 179 | Yes | n.d. |
| 9B8 | 85 | 183 | No | n.d. |
| 13C2 | 94 | 379 | No | n.d. |
| 14A9 | 148 | 581 | No | n.d. |
| 6D5 | 210 | 647 | No | n.d. |
| 1C5 | 296 | 652 | No | n.d. |

Note:
Antibody 4901 is commercially available (Sigma, Product No. C7113).
n.d. = not determined

TABLE 3
Characteristics of the anti-CGRP monoclonal antibodies'
binding to rat α-CGRP and antagonist activity

| | | Cell-based blocking | |
|---|---|---|---|
| | | of binding of rat | |
| | | α-CGRP to its | In vivo |
| | K_{D} to rat | receptor at 25° C. | blocking in |
| Anti- | α-CGRP | (measured by | saphenous |
| bodies | at 37° C. (nM) | cAMP activation) | nerve assay |
| 4901 | 3.4 | Yes | Yes |
| 7E9 | 47 | Yes | Yes |
| 6H2 | 54 | No | No |
| 8B6 | 75 | Yes | Yes |
| 7D11 | 218 | Yes | Yes |
| 10A8 | 451 | No | n.d. |
| 9B8 | 876 | No | n.d. |
| 14E10 | 922 | No | n.d. |
| 13C2 | >1000 | No | n.d. |
| 14A9 | >1000 | No | n.d. |
| 6D5 | >1000 | No | n.d. |
| 1C5 | >1000 | No | n.d. |

“n.d.” indicates no test was performed for the antibody.

TABLE 4
Amino acid sequences of human α-CGRP fragments (SEQ ID NOS: 15-
40) and related peptides (SEQ ID NOS: 41-47). All peptides are
C-terminally amidated except SEQ ID NOS:36-40. Underlined
residues indicate point mutations.

| | | SEQ |
|---|---|---|
| | | ID |
| CGRP | Amino acid sequence | NO |
| 1-37 (WT) | ACDTATCVTHRLAGLLSRSGGVVKNNFVPTNVGSKAF | 15 |
| 8-37 | VTHRLAGLLSRSGGVVKNNFVPTNVGSKAF | 16 |
| 19-37 | SGGVVKNNFVPTNVGSKAF | 17 |
| P29A (19-37) | SGGVVKNNFVATNVGSKAF | 18 |
| K35A (19-37) | SGGVVKNNFVPTNVGSAAF | 19 |
| K35E (19-37) | SGGVVKNNFVPTNVGSEAF | 20 |
| K35M (19-37) | SGGVVKNNFVPTNVGSMAF | 21 |
| K35Q (19-37) | SGGVVKNNFVPTNVGSQAF | 22 |
| F37A (19-37) | SGGVVKNNFVPTNVGSKAA | 23 |
| 25-38A | NNFVPTNVGSKAFA | 24 |
| 25-37 | NNFVPTNVGSKAF | 25 |
| F27A (25-37) | NNAVPTNVGSKAF | 26 |
| V28A (25-37) | NNFAPTNVGSKAF | 27 |
| P29A (25-37) | NNFVATNVGSKAF | 28 |
| T30A (25-37) | NNFVPANVGSKAF | 29 |
| N31A (25-37) | NNFVPTAVGSKAF | 30 |
| V32A (25-37) | NNFVPTNAGSKAF | 31 |
| G33A (25-37) | NNFVPTNVASKAF | 32 |
| 534A (25-37) | NNFVPTNVGAKAF | 33 |
| F37A (25-37) | NNFVPTNVGSKAA | 34 |
| 26-37 | NFVPTNVGSKAF | 35 |
| 19-37-COOH | SGGVVKNNFVPTNVGSKAF | 36 |
| 19-36-COOH | SGGVVKNNFVPTNVGSKA | 37 |
| 1-36-COOH | ACDTATCVTHRLAGLLSRSGGVVKNNFVPTNVGSKA | 38 |
| 1-19-COOH | ACDTATCVTHRLAGLLSRS | 39 |
| 1-13-COOH | ACDTATCVTHRLA | 40 |
| rat α (1-37) | SCNTATCVTHRLAGLLSRSGGVVKDNFVPTNVGSEAF | 41 |
| rat α (19-37) | SGGVVKDNFVPTNVGSEAF | 42 |
| human β (1-37) | ACNTATCVTHRLAGLLSRSGGMVKSNFVPTNVGSKAF | 43 |
| rat β (1-37) | SCNTATCVTHRLAGLLSRSGGVVKDNFVPTNVGSKAF | 44 |
| Human | CGNLSTCMLGTYTQDFNKFHTFPQTAIGVGAP | 45 |
| calcitonin | | |
| (1-32) | | |
| Human amylin | KCNTATCATQRLANFLVHSSNNFGAILSSTNVGSNTY | 46 |
| (1-37) | | |
| Human | YRQSMNNFQGLRSFGCRFGTCTVQKLAHQIYQFTDK | 47 |
| adrenomedullin | DKDNVAPRSKISPQGY | |
| (1-52) | | |

### Example 2

### Screening of Anti-CGRP Antagonist Antibodies Using In Vitro Assays

Murine anti-CGRP antibodies were further screened for antagonist activity in vitro using cell based cAMP activation assay and binding assay.

Antagonist activity measured by cAMP assay. Five microliters of human or rat α-CGRP (final concentration 50 nM) in the presence or absence of an anti-CGRP antibody (final concentration 1-3000 nM), or rat α-CGRP or human α-CGRP (final concentration 0.1 nM-10 μM; as a positive control for ε-AMP activation) was dispensed into a 384-well plate (Nunc, Cat. No. 264657). Ten microliters of cells (human SK-N-MC if human α-CGRP is used, or rat L6 from ATCC if rat α-CGRP is used) in stimulation buffer (20 mM HEPES, pH 7.4, 146 mM NaCl, 5 mM KCl, 1 mM CaCl₂, 1 mM MgCl₂, and 500 uM 3-Isobutyl-1-methylxanthine (IBMX)) were added into the wells of the plate. The plate was incubated at room temperature for 30 min.

After the incubation, cAMP activation was performed using HitHunter™ Enzyme Fragment Complementation Assay (Applied Biosystems) following manufacture's instruction. The assay is based on a genetically engineered β-galactosidase enzyme that consists of two fragments—termed Enzyme Acceptor (EA) and Enzyme Donor (ED). When the two fragments are separated, the enzyme is inactive. When the fragments are together they can recombine spontaneously to form active enzyme by a process called complementation. The EFC assay platform utilizes an ED-cAMP peptide conjugate in which cAMP is recognized by anti-cAMP. This ED fragment is capable of reassociation with EA to form active enzyme. In the assay, anti-cAMP antibody is optimally titrated to bind ED-cAMP conjugate and inhibit enzyme formation. Levels of cAMP in cell lysate samples compete with ED-cAMP conjugate for binding to the anti-cAMP antibody. The amount of free ED conjugate in the assay is proportional to the concentration of cAMP. Therefore, cAMP is measured by the formation of active enzyme that is quantified by the turnover of β-galactosidase luminescent substrate. The CAMP activation assay was performed by adding 10 μl of lysis buffer and anti-cAMP antibody (1:1 ratio) following by incubation at room temperature for 60 min. Then 10 μl of ED-cAMP reagent was added into each well and incubated for 60 minutes at room temperature. After the incubation, 20 μl of EA reagent and CL mixture (containing the substrate) (1:1 ratio) was added into each well and incubated for 1-3 hours or overnight at room temperature. The plate was read at 1 second/well on PMT instrument or 30 seconds/place on imager. The antibodies that inhibit activation of cAMP by α-CGRP were identified (referred to as “yes”) in Tables 2 and 3 above. Data in Tables 2 and 3 indicate that antibodies that demonstrated antagonist activity in the assay generally have high affinity. For example, antibodies having K_{D} (determined at 25° C.) of about 80 nM or less to human α-CGRP or having K_{D} (determined at 37° C.) of about 47 nM or less to rat α-CGRP showed antagonist activity in this assay.

Radioligand binding assay. Binding assay was performed to measure the IC₅₀ of anti-CGRP antibody in blocking the CGRP from binding to the receptor as described previously. Zimmermann et al., Peptides 16:421-4, 1995; Mallee et al., J. Biol. Chem. 277:14294-8, 2002. Membranes (25 μg) from SK-N-MC cells were incubated for 90 min at room temperature in incubation buffer (50 mM Tris-HCL, pH 7.4, 5 mM MgCL₂, 0.1% BSA) containing 10 pM ¹²⁵I-human α-CGRP in a total volume of 1 mL. To determine inhibition concentrations (IC₅₀), antibodies or unlabeled CGRP (as a control), from a about 100 fold higher stock solution were dissolved at varying concentrations in the incubation buffer and incubated at the same time with membranes and 10 pM ¹²⁵I-human α-CGRP. Incubation was terminated by filtration through a glass microfiber filter (GF/B, 1 μm) which had been blocked with 0.5% polyethylemimine. Dose response curves were plotted and Kᵢ values were determined by using the equation: Kᵢ=IC₅₀/(1+([ligand]/K_{D}); where the equilibrium dissociation constant K_{D}=8 pM for human α-CGRP to CGRP1 receptor as present in SK-N-MC cells, and Bₘₐₓ=0.025 pmol/mg protein. The reported IC₅₀ value (in terms of IgG molecules) was converted to binding sites (by multiplying it by 2) so that it could be compared with the affinities (K_{D}) determined by Biacore (see Table 2).

Table 2 shows the IC₅₀ of murine antibodies 7E9, 8B6, 6H2 and 4901. Data indicate that antibody affinity generally correlates with IC₅₀: antibodies with higher affinity (lower K_{D} values) have lower IC₅₀ in the radioligand binding assay.

### Example 3

### Effect of Anti-CGRP Antagonist Antibodies on Skin Vasodilatation Induced by Stimulation of Rat Saphenous Nerve

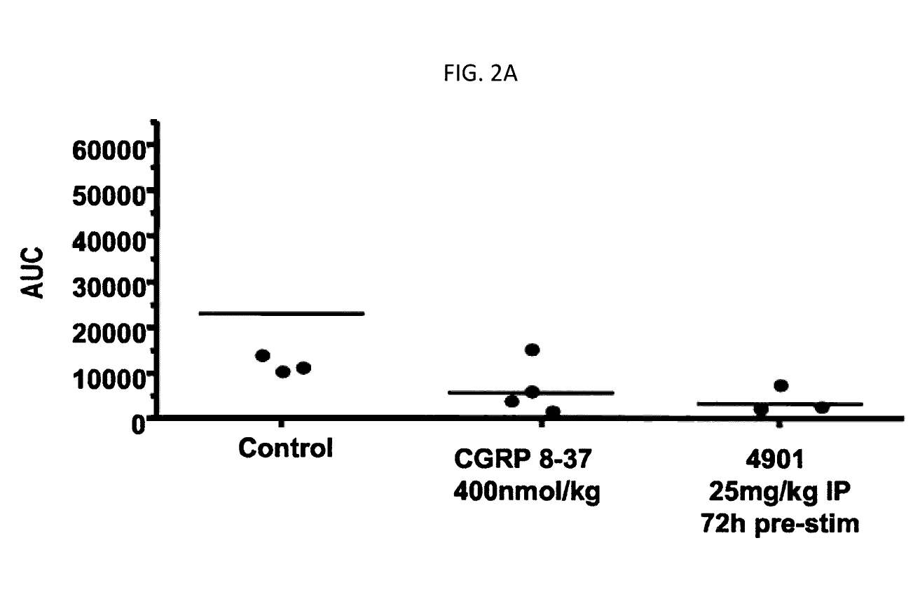

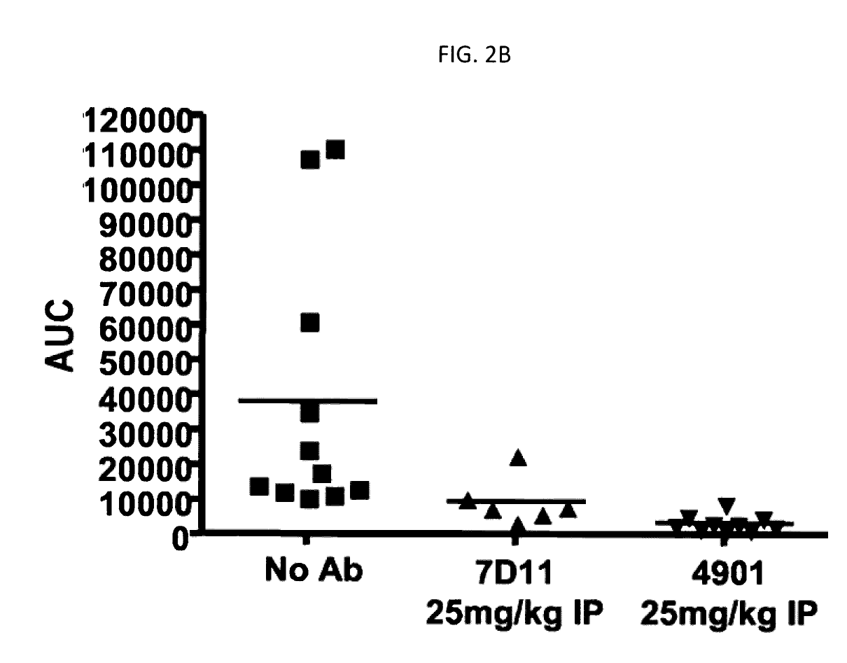

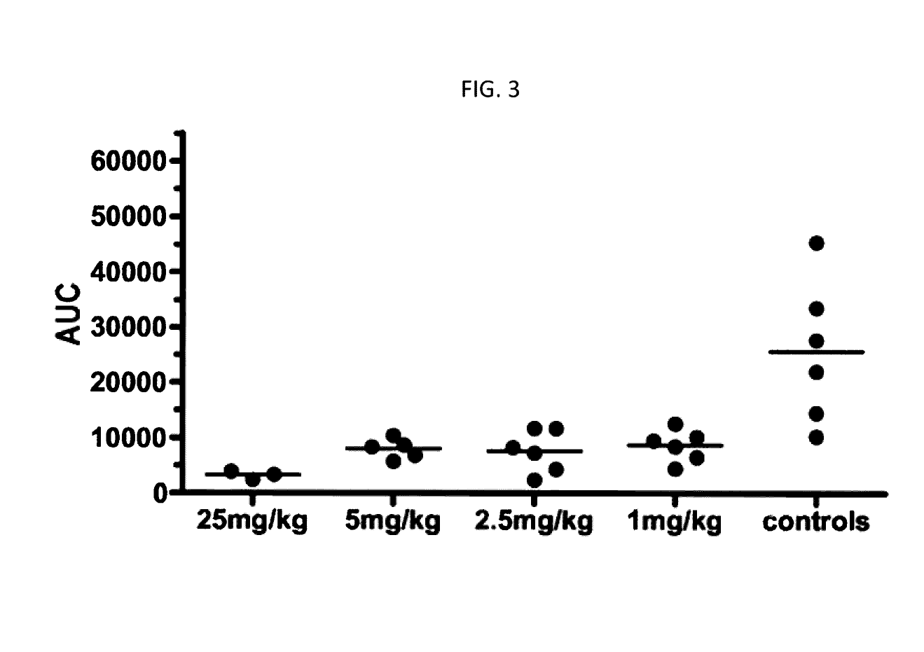

To test antagonist activity of anti-CGRP antibodies, effect of the antibodies on skin vasodilatation by stimulation of rat saphenous nerve was tested using a rat model described previously. Escott et al., Br. J. Pharmacol. 110:772-776, 1993. In this rat model, electrical stimulation of saphenous nerve induces release of CGRP from nerve endings, resulting in an increase in skin blood flow. Blood flow in the foot skin of male Sprague Dwaley rats (170-300 g, from Charles River Hollister) was measured after saphenous nerve stimulation. Rats were maintained under anesthesia with 2% isoflurane. Bretylium tosylate (30 mg/kg, administered i.v.) was given at the beginning of the experiment to minimize vasoconstriction due to the concomitant stimulation of sympathetic fibers of the saphenous nerve. Body temperature was maintained at 37° C. by the use of a rectal probe thermostatically connected to a temperature controlled heating pad. Compounds including antibodies, positive control (CGRP 8-37), and vehicle (PBS, 0.01% Tween 20) were given intravenously through the right femoral vein, except for the experiment shown in FIG. 3, the test compound and the control were injected through tail vein, and for experiments shown in FIGS. 2A and 2B, antibodies 4901 and 7D11 were injected intraperitoneally (IP). Positive control compound CGRP 8-37 (vasodilatation antagonist), due to its short half-life, was given 3-5 min before nerve stimulation at 400 nmol/kg (200 μl). Tan et al., Clin. Sci. 89:656-73, 1995. The antibodies were given in different doses (1 mg/kg, 2.5 mg/kg, 5 mg/kg, 10 mg/kg, and 25 mg/kg).

For experiments shown in FIGS. 2A and 2B, antibody 4901 (25 mg/kg), antibody 7D11 (25 mg/kg), or vehicle control (PBS with 0.01% Tween 20) was administered intraperitoneally (IP) 72 hours before the electrical pulse stimulation. For experiment shown in FIG. 3, antibody 4901 (1 mg/kg, 2.5 mg/kg, 5 mg/kg, or 25 mg/kg) or vehicle control (PBS with 0.01% Tween 20) was administered intravenously 24 hours before the electrical pulse stimulation. After administration of the antibodies or vehicle control, the saphenous nerve of the right hindlimb was exposed surgically, cut proximally and covered with plastic wrap to prevent drying. A laser Doppler probe was placed over the medio-dorsal side of the hindpaw skin, which is the region innervated by the saphenous nerve. Skin blood flow, measured as blood cell flux, was monitored with a laser Doppler flow meter. When a stable base-line flux (less than 5% variation) was established for at least 5 min, the nerve was placed over platinum bipolar electrodes and electrically stimulated with 60 pulses (2 Hz, 10 V, 1 ms, for 30 sec) and then again 20 minutes later. Cumulative change in skin blood flow was estimated by the area under the flux-time curve (AUC, which is equal to change in flux multiplied by change in time) for each flux response to electrical pulse stimulation. The average of the blood flow response to the two stimulations was taken. Animals were kept under anesthesia for a period of one to three hours.

As shown in FIG. 2A and FIG. 2B, blood flow increase stimulated by applying electronic pulses on saphenous nerve was inhibited by the presence of CGRP 8-37 (400 nmol/kg, administered i.v.), antibody 4901 (25 mg/kg, administered ip), or antibody 7D11 (25 mg/kg, administered ip) as compared to the control. CGRP 8-37 was administered 3-5 min before the saphenous nerve stimulation; and antibodies were administered 72 hours before the saphenous nerve stimulation. As shown in FIG. 3, blood flow increase stimulated by applying electronic pulses on saphenous nerve was inhibited by the presence of antibody 4901 at different doses (1 mg/kg, 2.5 mg/kg, 5 mg/kg, and 25 mg/kg) administered intravenously at 24 h before the saphenous nerve stimulation.

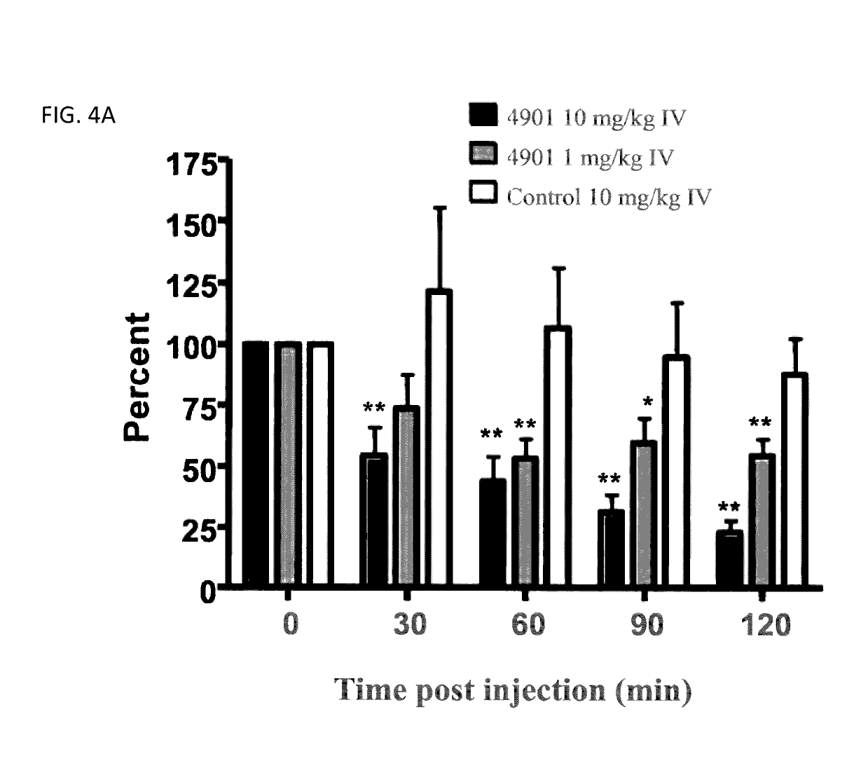

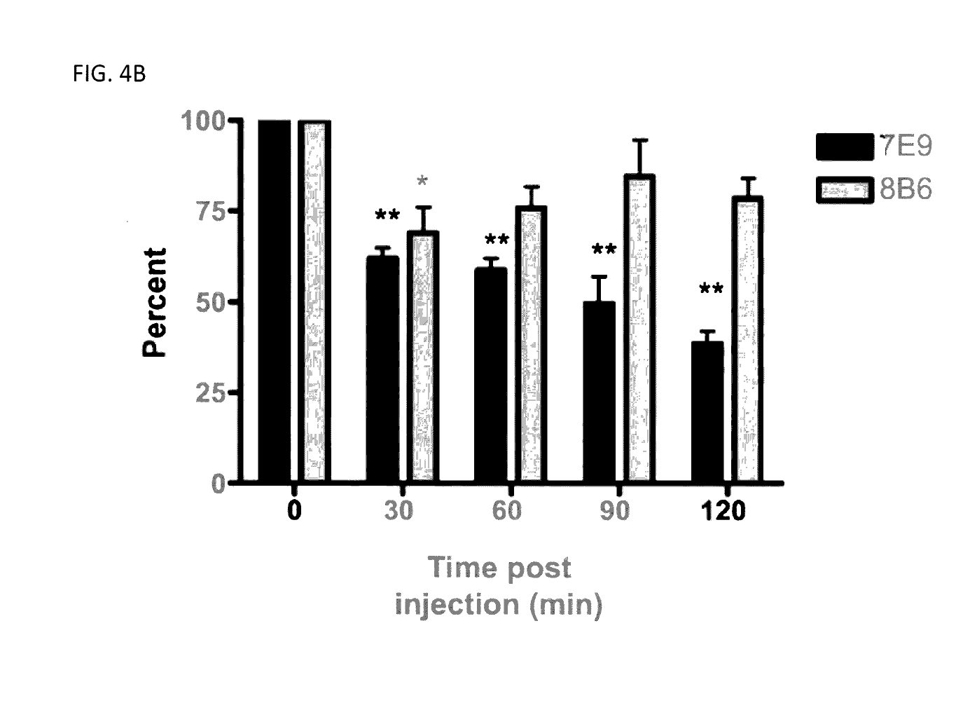

For experiments shown in FIGS. 4A and 4B, saphenous nerve was exposed surgically before antibody administration. The saphenous nerve of the right hindlimb was exposed surgically, cut proximally and covered with plastic wrap to prevent drying. A laser Doppler probe was placed over the medio-dorsal side of the hindpaw skin, which is the region innervated by the saphenous nerve. Skin blood flow, measured as blood cell flux, was monitored with a laser Doppler flow meter. Thirty to forty five minutes after bretylium tosylate injection, when a stable base-line flux (less than 5% variation) was established for at least 5 min, the nerve was placed over platinum bipolar electrodes and electrically stimulated (2 Hz, 10V, 1 ms, for 30 sec) and again 20 minutes later. The average of the blood flow flux response to these two stimulations was used to establish the baseline response (time 0) to electrical stimulation. Antibody 4901 (1 mg/kg or 10 mg/kg), antibody 7E9 (10 mg/kg), antibody 8B6 (10 mg/kg), or vehicle (PBS with 0.01% Tween 20) were then administered intravenously (i.v.). The nerve was subsequently stimulated (2 Hz, 10V, 1 ms, for 30 sec) at 30 min, 60 min, 90 min, and 120 min after antibody or vehicle administration. Animals were kept under anesthesia for a period of approximately three hours. Cumulative change in skin blood flow was estimated by the area under the flux-time curve (AUC, which is equal to change in flux multiplied by change in time) for each flux response to electrical pulse stimulations.

As shown in FIG. 4A, blood flow increase stimulated by applying electronic pulses on saphenous nerve was significantly inhibited by the presence of antibody 4901 1 mg/kg administered i.v., when electronic pulse stimulation was applied at 60 min, 90 min, and 120 min after the antibody administration, and blood flow increase stimulated by applying electronic pulses on saphenous nerve was significantly inhibited by the presence of antibody 4901 10 mg/kg administered i.v., when electronic pulse stimulation was applied at 30 min, 60 min, 90 min, and 120 min after the antibody administration. FIG. 4B shows that blood flow increase stimulated by applying electronic pulses on saphenous nerve was significantly inhibited by the presence of antibody 7E9 (10 mg/kg, administered i.v.) when electronic pulse stimulation was applied at 30 min, 60 min, 90 min, and 120 min after antibody administration, and by the presence of antibody 8B6 (10 mg/kg, administered i.v.) when electronic pulse stimulation was applied at 30 min after antibody administration.

These data indicate that antibodies 4901, 7E9, 7D11, and 8B6 are effective in blocking CGRP activity as measured by skin vasodilatation induced by stimulation of rat saphenous nerve.

### Example 4

### Characterization of Anti-CGRP Antibody G1 and its Variants

Amino acid sequences for the heavy chain variable region and light chain variable region of anti-CGRP antibody G1 are shown in FIG. 5. The following methods were used for expression and characterization of antibody G1 and its variants.

Expression vector used. Expression of the Fab fragment of the antibodies was under control of an IPTG inducible lacZ promoter similar to that described in Barbas (2001) Phage display: a laboratory manual, Cold Spring Harbor, N.Y., Cold Spring Harbor Laboratory Press pg 2.10. Vector pComb3X), however, modifications included addition and expression of the following additional domains: the human Kappa light chain constant domain and the CH1 constant domain of IgG2 human immunoglobulin, Ig gamma-2 chain C region, protein accession number P01859; Immunoglobulin kappa light chain (homosapiens), protein accession number CAA09181.

Small scale Fab preparation. From E. coli transformed (either using electroporation-competent TG1 cells or chemically-competent Top 10 cells) with a Fab library, single colonies were used to inoculate both a master plate (agar LB+carbenicillin (50 ug/mL)+2% glucose) and a working plate (2 mL/well, 96-well/plate) where each well contained 1.5 mL LB+carbenicillin (50 ug/mL)+2% glucose. A gas permeable adhesive seal (ABgene, Surrey, UK) was applied to the plate. Both plates were incubated at 30° C. for 12-16 h; the working plate was shaken vigorously. The master plate was stored at 4° C. until needed, while the cells from the working plate were pelleted (4000 rpm, 4° C., 20 mins) and resuspended in 1.0 mL LB+carbenicillin (50 ug/mL)+0.5 mM IPTG to induce expression of Fabs by vigorous shaking for 5 h at 30° C. Induced cells were centrifuges at 4000 rpm, 4° C. for 20 mins and resuspended in 0.6 mL Biacore HB-SEP buffer (10 mM Hepes pH 7.4, 150 mM NaCl, 3 mM EDTA, 0.005% v/v P20). Lysis of HB-SEP resuspended cells was accomplished by freezing (−80° C.) and then thawing at 37° C. Cell lysates were centrifuged at 4000 rpm, 4° C. for 1 hour to separate the debris from the Fab-containing supernatants, which were subsequently filtered (0.2 um) using a Millipore MultiScreen Assay System 96-Well Filtration Plate and vacuum manifold. Biacore was used to analyze filtered supernatants by injecting them across CGRPs on the sensor chip. Affinity-selected clones expressing Fabs were rescued from the master plate, which provided template DNA for PCR, sequencing, and plasmid preparation.

Large scale Fab preparation. To obtain kinetic parameters, Fabs were expressed on a larger scale as follows. Erlenmeyer flasks containing 150 mL LB+carbenicillin (50 ug/mL)+2% glucose were inoculated with 1 mL of a “starter” overnight culture from an affinity-selected Fab-expressing E. coli clone. The remainder of the starter culture (˜3 mL) was used to prepare plasmid DNA (QIAprep mini-prep, Qiagen kit) for sequencing and further manipulation. The large culture was incubated at 30° C. with vigorous shaking until an OD600 nm of 1.0 was attained (typically 12-16 h). The cells were pelleted by centrifuging at 4000 rpm, 4° C. for 20 mins, and resuspended in 150 mL LB+carbenicillin (50 ug/mL)+0.5 mM IPTG. After 5 h expression at 30° C., cells were pelleted by centrifuging at 4000 rpm, 4° C. for 20 mins, resuspended in 10 mL Biacore HBS-EP buffer, and lysed using a single freeze (−80° C.)/thaw (37° C.) cycle. Cell lysates were pelleted by centrifuging at 4000 rpm, 4° C. for 1 hour, and the supernatant was collected and filtered (0.2 um). Filtered supernatants were loaded onto Ni-NTA superflow sepharose (Qiagen, Valencia. Calif.) columns equilibrated with PBS, pH 8, then washed with 5 column volumes of PBS, pH 8. Individual Fabs eluted in different fractions with PBS (pH 8)+300 mM Imidazole. Fractions containing Fabs were pooled and dialyzed in PBS, then quantified by ELISA prior to affinity characterization.

Full antibody preparation. For expression of full antibodies, heavy and light chain variable regions were cloned in mammalian expression vectors and transfected using lipofectamine into HEK 293 cells for transient expression. Antibodies were purified using protein A using standard methods.

Vector pDb.CGRP.hFcGI is an expression vector comprising the heavy chain of the G1 antibody, and is suitable for transient or stable expression of the heavy chain. Vector pDb.CGRP.hFcGI has nucleotide sequences corresponding to the following regions: the murine cytomegalovirus promoter region (nucleotides 7-612); a synthetic intron (nucleotides 613-1679); the DHFR coding region (nucleotides 688-1253); human growth hormone signal peptide (nucleotides 1899-1976); heavy chain variable region of G1 (nucleotides 1977-2621); human heavy chain IgG2 constant region containing the following mutations: A330P331 to S330S331 (amino acid numbering with reference to the wildtype IgG2 sequence; see Eur. J. Immunol. (1999) 29:2613-2624). Vector pDb.CGRP.hFcGI was deposited at the ATCC on Jul. 15, 2005, and was assigned ATCC Accession No. PTA-6867.

Vector pEb.CGRP.hKGI is an expression vector comprising the light chain of the G1 antibody, and is suitable for transient expression of the light chain. Vector pEb.CGRP.hKGI has nucleotide sequences corresponding to the following regions: the murine cytomegalovirus promoter region (nucleotides 2-613); human EF-1 intron (nucleotides 614-1149); human growth hormone signal peptide (nucleotides 1160-1237); antibody G1 light chain variable region (nucleotides 1238-1558); human kappa chain constant region (nucleotides 1559-1882). Vector pEb.CGRP.hKGI was deposited at the ATCC on Jul. 15, 2005, and was assigned ATCC Accession No. PTA-6866.

Biacore assay for affinity determination. Affinities of G1 monoclonal antibody and its variants were determined at either 25° C. or 37° C. using the Biacore3000™ surface plasmon resonance (SPR) system (Biacore, INC, Piscataway N.J.). Affinity was determined by capturing N-terminally biotinylated CGRP or fragments via pre-immobilized streptavidin (SA sensor chip) and measuring the binding kinetics of antibody G1 Fab fragments or variants titrated across the CGRP or fragment on the chip. All Biacore assays were conducted in HBS-EP running buffer (10 mM HEPES pH 7.4, 150 mM NaCl, 3 mM EDTA, 0.005% v/v polysorbate P20). CGRP surfaces were prepared by diluting the N-biotinylated CGRP to a concentration of less than 0.001 mg/mL into HBS-EP buffer and injecting it across the SA sensor chip using variable contact times. Low capacity surfaces, corresponding to capture levels <50 response units (RU) were used for high-resolution kinetic studies, whereas high capacity surfaces (about 800 RU of captured CGRP) were used for concentration studies, screening, and solution affinity determinations. Kinetic data were obtained by diluting antibody G1 Fab serially in two- or three-fold increments to concentrations spanning 1 uM-0.1 nM (aimed at 0.1-10× estimated K_{D}). Samples were typically injected for 1 minute at 100 μL/min and dissociation times of at least 10 minutes were allowed. After each binding cycle, surfaces were regenerated with 25 mM NaOH in 25% v/v ethanol, which was tolerated over hundreds of cycles. An entire titration series (typically generated in duplicate) was fit globally to a 1:1 Langmuir binding model using the BIAevaluation program. This returned a unique pair of association and dissociation kinetic rate constants (respectively, kₒₙ and k_{off}) for each binding interaction, whose ratio gave the equilibrium dissociation constant (K_{D}=k_{off}/kₒₙ). Affinities (K_{D} values) determined in this way are listed in Tables 6 and 7.

High-resolution analysis of binding interactions with extremely slow offrates. For interactions with extremely slow offrates (in particular, antibody G1 Fab binding to human α-CGRP on the chip at 25° C.), affinities were obtained in a two-part experiment. The protocol described above was used with the following modifications. The association rate constant (kₒₙ) was determined by injecting a 2-fold titration series (in duplicate) spanning 550 nM-1 nM for 30 sec at 100 uL/min and allowing only a 30 sec dissociation phase. The dissociation rate constant (k_{off}) was determined by injecting three concentrations (high, medium, and low) of the same titration series in duplicate for 30 sec and allowing a 2-hour dissociation phase. The affinity (K_{D}) of each interaction was obtained by combining the kₒₙ and k_{off} values obtained in both types of experiments, as shown in Table 5.

Determining solution affinity by Biacore. The solution affinity of antibody G1 for rat α-CGRP and F37A (19-37) human α-CGRP was measured by Biacore at 37° C. A high capacity CGRP chip surface was used (the high-affinity human α-CGRP was chosen for detection purposes) and HBS-EP running buffer was flowed at 5 uL/min. Antibody G1 Fab fragment at a constant concentration of 5 nM (aimed to be at or below the expected K_{D} of the solution-based interaction) was pre-incubated with competing peptide, either rat α-CGRP or F37A (19-37) human α-CGRP, at final concentrations spanning 1 nM to 1 uM in 3-fold serial dilutions. Antibody G1 Fab solutions in the absence or presence of solution-based competing peptide, were injected across CGRP on the chip and the depletion of binding responses detected at the chip surface as a result of solution competition was monitored. These binding responses were converted to “free Fab concentrations” using a calibration curve, which was constructed by titrating antibody G1 Fab alone (5, 2.5, 1.25, 0.625, 0.325 and 0 nM) across the CGRP on the chip. “Free Fab concentrations” were plotted against the concentration of competing solution-based peptide used to generate each data point and fit to a solution affinity model using the BIAevaluation software. The solution affinities determined (indirectly) in this way are shown in Tables 5 and 7 and were used to validate the affinities obtained when Fabs are injected directly across N-biotinylated CGRPs on a SA chip. The close agreement between the affinities determined by these two methods confirms that tethering an N-biotinylated version of the CGRP to the chip does not alter its native solution binding activity.

Table 5 below shows the binding affinities of antibody G1 to human α-CGRP, human β-CGRP, rat α-CGRP, and rat β-CGRP determined by Biacore, by flowing Fab fragments across N-biotinylated CGRPs on a SA chip. To better resolve the affinities of binding interactions with extremely slow offrates, affinities were also determined in a two-part experiment to complement this assay orientation, the solution affinity of the rat α-CGRP interaction was also determined (as described above). The close agreement of the affinities measured in both assay orientations confirms that the binding affinity of the native rat α-CGRP in solution is not altered when it is N-biotinylated and tethered to a SA chip.

TABLE 5
Binding affinities of antibody G1 Fabs
titrated across CGRPs on the chip

| | Temp. | | | |
|---|---|---|---|---|
| CGRP on chip | (° C.) | kₒₙ (1/Ms) | k_{off} (1/s) | K_{D} (nM) |
| Human α-CGRP | 25 | 1.86 × 10⁵ | 7.80 × 10⁻⁶ | 0.042 |
| | | | | (7%, n = 4)* |
| Human α-CGRP | 37 | 5.78 × 10⁵ | 3.63 × 10⁻⁵ | 0.063 |
| | | | | (4%, n = 2)* |
| Human β-CGRP | 37 | 4.51 × 10⁵ | 6.98 × 10⁻⁵ | 0.155 |
| Rat α-CGRP | 25 | 5.08 × 10⁴ | 6.18 × 10⁻⁵ | 1.22 |
| | | | | (12%, n = 2)* |
| Rat α-CGRP | 37 | 1.55 × 10⁵ | 3.99 × 10⁻⁴ | 2.57* |
| | | | | (Solution K_{D} = 10 |
| | | | | (50%, n = 4)** |
| Rat β-CGRP | 37 | 5.16 × 10⁵ | 7.85 × 10⁻⁵ | 0.152 |

*Affinities for α-CGRPs (rat and human) were determined in a high-resolution two-part experiment, in which the dissociation phase was monitored for 2 hours (the values for kₒₙ, k_{off}, and K_{D} represent the average of n replicate experiments with the standard deviation expressed as a percent variance). Affinities for β-CGRPs (rat and human) were determined by global analysis using only a 20-min dissociation phase, which was not accurate enough to quantify their extremely offrates (their offrates are likely slower than stated here and therefore their affinities are likely even higher). Antibody G1 Fab dissociated extremely slowly from all CGRPs (except α-rat CGRP) with offrates that approached the resolution limit of the Biacore assay (especially at 25° C.).
**Solution affinity determined by measuring the depletion of binding responses detected at CGRP on the chip for antibody G1 Fab pre-incubated with solution-based rat α-CGRP competitor.

Table 6 below shows antibodies having the amino acid sequence variation as compared to antibody G1 and their affinities to both rat α-CGRP and human α-CGRP. All amino acid substitutions of the variants shown in Table 6 are described relative to the sequence of G1. The binding affinities of Fab fragments were determined by Biacore by flowing them across CGRPs on a SA chip.

TABLE 6
Amino acid sequences and binding affinity data for antibody
G1 variants determined at 37° C. by Biacore.

| | | | | | α-rat | α-rat | α-human | α-human |
|---|---|---|---|---|---|---|---|---|
| Clone | L1 | L2 | H2 | HC-FW3 | k_{off} (1/s) | K_{D} (nM) | k_{off} (1/s) | K_{D} (nM) |
| G1 | | | | | 3.99 × 10⁻⁴ | 2.57 | 3.63 × 10⁻⁵ | 0.063 |
| M1 | | | | A100L | 1.10 × 10⁻³ | | 1.73 × 10⁻⁴ | |
| M2 | | | | L99A | 2.6 × 10⁻³ | 58 | 3.1 × 10⁻⁴ | 3 |
| | | | | A100R | | | | |
| M3 | | | | L99A | 2.0 × 10⁻³ | 61 | 2.1 × 10⁻⁴ | 1.7 |
| | | | | A100S | | | | |
| M4 | | | | L99A | 1.52 × 10⁻³ | 84.4 | 6.95 × 10⁻⁵ | 0.43 |
| | | | | A100V | | | | |
| M5 | | | | L99A | 7.35 × 10⁻⁴ | 40.8 | 3.22 × 10⁻⁵ | 0.20 |
| | | | | A100Y | | | | |
| M6 | | | | L99N | 7.84 × 10⁻⁴ | 43.6 | 1.33 × 10⁻⁴ | 0.83 |
| M7 | | | | L99N | 9.18 × 10⁻⁴ | 51.0 | 2.43 × 10⁻⁴ | 1.52 |
| | | | | A100C | | | | |
| M8 | | | | L99N | 7.45 × 10⁻⁴ | 41.4 | 9.20 × 10⁻⁵ | 0.58 |
| | | | | A100G | | | | |
| M9 | | | | L99N | n.d. | n.d. | 1.00 × 10⁻⁵ | 0.06 |
| | | | | A100Y | | | | |
| M10 | | | | L99S | 1.51 × 10⁻³ | 83.9 | 1.73 × 10⁻⁴ | 1.08 |
| | | | | A100S | | | | |
| M11 | | | | L99S | 4.83 × 10⁻³ | 268.3 | 2.83 × 10⁻⁴ | 1.77 |
| | | | | A100T | | | | |
| M12 | | | | L99S | 1.94 × 10⁻³ | 107.8 | 1.01 × 10⁻⁴ | 0.63 |
| | | | | A100V | | | | |
| M13 | | | | L99T | 1.84 × 10⁻³ | 102.2 | 1.86 × 10⁻⁴ | 1.16 |
| | | | | A100G | | | | |
| M14 | | | | L99T | n.d. | n.d. | 1.00 × 10⁻⁵ | 0.06 |
| | | | | A100K | | | | |
| M15 | | | | L99T | 1.15 × 10⁻³ | 63.9 | 1.58 × 10⁻⁵ | 0.10 |
| | | | | A100P | | | | |
| M16 | | | | L99T | 9.96 × 10⁻⁴ | 55.3 | 1.65 × 10⁻⁴ | 1.03 |
| | | | | A100S | | | | |
| M17 | | | | L99T | 2.06 × 10⁻³ | 114.4 | 1.85 × 10⁻⁴ | 1.16 |
| | | | | A100V | | | | |
| M18 | | | | L99V | 1.22 × 10⁻³ | 67.8 | 7.03 × 10⁻⁵ | 0.44 |
| | | | | A100G | | | | |
| M19 | | | | L99V | n.d. | n.d. | 1.00 × 10⁻⁵ | 0.06 |
| | | | | A100R | | | | |
| M20 | R28W | | | L99R | 1.44 × 10⁻³ | 80.0 | 1.36 × 10⁻⁴ | 0.85 |
| | | | | A100L | | | | |
| M21 | R28W | | | L99S | 6.95 × 10⁻⁴ | 15.2 | 1.42 × 10⁻⁴ | 1.23 |
| M22 | R28W | | | L99T | 1.10 × 10⁻³ | 61.1 | 1.16 × 10⁻⁴ | 0.73 |
| M23 | R28G | | | L99T | 7.99 × 10⁻⁴ | 44.4 | 1.30 × 10⁻⁴ | 0.81 |
| | | | | A100V | | | | |
| M24 | R28L | | | L99T | 1.04 × 10⁻³ | 57.8 | 1.48 × 10⁻⁴ | 0.93 |
| | | | | A100V | | | | |
| M25 | R28N | | | L99T | 1.4 × 10⁻³ | 76 | 1.4 × 10⁻⁴ | 1.3 |
| | | | | A100V | | | | |
| M26 | R28N | | A57G | L99T | 9.24 × 10⁻⁴ | 51.3 | 1.48 × 10⁻⁴ | 0.93 |
| | | | | A100V | | | | |
| M27 | R28N | | | L99T | 3.41 × 10⁻³ | 189.4 | 3.57 × 10⁻⁴ | 2.23 |
| | T30A | | | A100V | | | | |
| M28 | R28N | | E54R | L99T | 1.25 × 10⁻³ | 69.4 | 9.96 × 10⁻⁵ | 0.62 |
| | T30D | | A57N | A100V | | | | |
| M29 | R28N | | | L99T | 3.59 × 10⁻³ | 199.4 | 3.80 × 10⁻⁴ | 2.38 |
| | T30G | | | A100V | | | | |
| M30 | R28N | | E54K | L99T | 6.38 × 10⁻³ | 354.4 | 5.90 × 10⁻⁴ | 3.69 |
| | T30G | | A57E | A100V | | | | |
| M31 | R28N | | E54K | L99T | 3.61 × 10⁻³ | 200.6 | 3.47 × 10⁻⁴ | 2.17 |
| | T30G | | A57G | A100V | | | | |
| M32 | R28N | | E54K | L99T | 2.96 × 10⁻³ | 164.4 | 2.71 × 10⁻⁴ | 1.69 |
| | T30G | | A57H | A100V | | | | |
| M33 | R28N | | E54K | L99T | 9.22 × 10⁻³ | 512.2 | 7.50 × 10⁻⁴ | 4.69 |
| | T30G | | A57N | A100V | | | | |
| | | | S58G | | | | | |
| M34 | R28N | | E54K | L99T | 2.17 × 10⁻³ | 120.6 | 6.46 × 10⁻⁴ | 4.04 |
| | T30G | | A57N | A100V | | | | |
| | | | S58T | | | | | |
| M35 | R28N | | E54K | L99T | 3.99 × 10⁻³ | 221.7 | 3.39 × 10⁻⁴ | 2.12 |
| | T30G | | A57S | A100V | | | | |
| M36 | R28N | | | L99T | 4.79 × 10⁻³ | 266.1 | 2.39 × 10⁻⁴ | 1.49 |
| | T30R | | | A100V | | | | |
| M37 | R28N | | A57G | L99T | 1.45 × 10⁻³ | 80.6 | 2.26 × 10⁻⁴ | 1.41 |
| | T30S | | | A100V | | | | |
| M38 | R28N | | | L99T | 5.11 × 10⁻³ | 283.9 | 2.18 × 10⁻⁴ | 1.36 |
| | T30W | | | A100V | | | | |
| M39 | R28N | G50A | A57N | L99T | 9.95 × 10⁻³ | 552.8 | 4.25 × 10⁻⁴ | 2.66 |
| | | L56T | S58Y | A100V | | | | |
| M40 | R28N | G50A | E54K | L99T | 0.36 | 20000.0 | 1.28 × 10⁻³ | 8.00 |
| | | L56T | A57L | A100V | | | | |
| M41 | R28N | G50A | E54K | L99T | 4.53 × 10⁻³ | 251.7 | 2.10 × 10⁻⁴ | 1.31 |
| | | L56T | A57N | A100V | | | | |
| | | | E64D | | | | | |
| M42 | R28N | G50A | E54K | L99T | 7.52 × 10⁻³ | 417.8 | 4.17 × 10⁻⁴ | 2.61 |
| | | L56T | A57N | A100V | | | | |
| | | | H61F | | | | | |
| M43 | R28N | G50A | E54K | L99T | 4.53 × 10⁻³ | 251.7 | 2.63 × 10⁻⁴ | 1.64 |
| | | L56T | A57N | A100V | | | | |
| | | | S58C | | | | | |
| M44 | R28N | G50A | E54K | L99T | 6.13 × 10⁻³ | 443 | 2.10 × 10⁻⁴ | 2.05 |
| | | L56T | A57N | A100V | | | | |
| | | | S58E | | | | | |
| M45 | R28N | G50A | E54K | L99T | 5.58 × 10⁻³ | 259 | 2.11 × 10⁻⁴ | 1.85 |
| | | L56T | A57N | A100V | | | | |
| | | | S58E | | | | | |
| | | | E64D | | | | | |
| M46 | R28N | G50A | E54K | L99T | 2.94 × 10⁻³ | 163.3 | 5.39 × 10⁻⁴ | 3.37 |
| | | L56T | A57N | A100V | | | | |
| | | | S58E | | | | | |
| | | | H61F | | | | | |
| M47 | R28N | G50A | E54K | L99T | 8.23 × 10⁻³ | 457.2 | 3.32 × 10⁻⁴ | 2.08 |
| | | L56T | A57N | A100V | | | | |
| | | | S58G | | | | | |
| M48 | R28N | G50A | E54K | L99T | 0.0343 | 1905.6 | 8.42 × 10⁻⁴ | 5.26 |
| | | L56T | A57N | A100V | | | | |
| | | | S58L | | | | | |
| M49 | R28N | G50A | E54K | L99T | 0.0148 | 822.2 | 5.95 × 10⁻⁴ | 3.72 |
| | | L56T | A57N | A100V | | | | |
| | | | S58Y | | | | | |
| | | | H61F | | | | | |
| M50 | R28N | G50A | E54K | L99T | 5.30 × 10⁻³ | 294.4 | 4.06 × 10⁻⁴ | 2.54 |
| | | L56T | A57R | A100V | | | | |
| M51 | R28N | L56I | E54K | L99T | 1.18 × 10⁻³ | 65.6 | 1.31 × 10⁻⁴ | 0.82 |
| | | | A57G | A100V | | | | |
| M52 | R28N | L56I | E54K | L99T | 2.29 × 10⁻³ | 127.2 | 2.81 × 10⁻⁴ | 1.76 |
| | | | A57N | A100V | | | | |
| | | | S58A | | | | | |
| M53 | R28N | L56I | E54K | L99T | 1.91 × 10⁻³ | 106.1 | 3.74 × 10⁻⁴ | 2.34 |
| | | | A57N | A100V | | | | |
| | | | S58G | | | | | |
| M54 | R28N | G50A | E54K | L99T | 2.16 × 10⁻³ | 120.0 | 1.79 × 10⁻³ | 11.19 |
| | T30A | | A57N | A100V | | | | |
| | | | S58P | | | | | |
| M55 | R28N | L56S | E54K | L99T | 5.85 × 10⁻³ | 325.0 | 4.78 × 10⁻⁴ | 2.99 |
| | T30A | | A57N | A100V | | | | |
| | | | S58E | | | | | |
| | | | E64D | | | | | |
| M56 | R28N | L56S | E54K | L99T | 9.35 × 10⁻³ | 519.4 | 4.79 × 10⁻⁴ | 2.99 |
| | T30D | | A57N | A100V | | | | |
| | | | H61F | | | | | |
| M57 | R28N | L56S | E54K | L99T | 0.0104 | 1.200 | 3.22 × 10⁻⁴ | 3.08 |
| | T30D | | A57N | A100V | | | | |
| | | | S58E | | | | | |
| M58 | R28N | L56S | E54K | L99T | No | n.d. | 1.95 × 10⁻³ | 12.19 |
| | T30D | | A57N | A100V | binding | | | |
| | | | S58I | | | | | |
| | | | H61F | | | | | |
| M59 | R28N | L56S | E54K | L99T | 0.0123 | 683.3 | 5.24 × 10⁻⁴ | 3.28 |
| | T30D | | A57N | A100V | | | | |
| | | | S58N | | | | | |
| | | | H61F | | | | | |
| M60 | R28N | L56S | E54K | L99T | 0.0272 | 1511.1 | 9.11 × 10⁻⁴ | 5.69 |
| | T30D | | A57N | A100V | | | | |
| | | | S58R | | | | | |
| | | | H61F | | | | | |
| M61 | R28N | A51H | E54Q | L99T | 5.21 × 10⁻³ | 289.4 | 4.59 × 10⁻⁴ | 2.87 |
| | T30G | | A57N | A100V | | | | |
| | | | H61F | | | | | |
| M62 | R28N | A51H | E54K | L99T | 5.75 × 10⁻³ | 242 | 5.57 × 10⁻⁴ | 5.86 |
| | T30G | L56T | A57N | A100V | | | | |
| | | | S58E | | | | | |
| M63 | R28N | G50A | E54K | L99T | 2.65 × 10⁻³ | 147.2 | 1.50 × 10⁻³ | 9.38 |
| | T30G | | A57N | A100V | | | | |
| | | | S58T | | | | | |
| M64 | R28N | G50A | E54K | L99T | 0.0234 | 1300.0 | 1.32 × 10⁻³ | 8.25 |
| | T30G | | A57N | A100V | | | | |
| | | | S58V | | | | | |
| M65 | R28N | G50A | E54K | L99T | 4.07 × 10⁻³ | 226.1 | 8.03 × 10⁻⁴ | 5.02 |
| | T30G | L56I | A57C | A100V | | | | |
| M66 | R28N | L56I | E54K | L99T | 5.11 × 10⁻³ | 283.9 | 5.20 × 10⁻⁴ | 3.25 |
| | T30G | | A57E | A100V | | | | |
| M67 | R28N | L56I | E54K | L99T | 1.71 × 10⁻³ | 95.0 | 8.20 × 10⁻⁴ | 5.13 |
| | T30G | | A57F | A100V | | | | |
| M68 | R28N | L56I | E54K | L99T | 6.76 × 10⁻³ | 375.6 | 4.28 × 10⁻⁴ | 2.68 |
| | T30G | | A57N | A100V | | | | |
| | | | S58D | | | | | |
| | | | E64D | | | | | |
| M69 | R28N | L56I | E54K | L99T | 1.81 × 10⁻³ | 100.6 | 7.33 × 10⁻⁴ | 4.58 |
| | T30G | | A57N | A100V | | | | |
| | | | S58E | | | | | |
| M70 | R28N | L56I | E54K | L99T | 6.07 × 10⁻³ | 337.2 | 5.59 × 10⁻⁴ | 3.49 |
| | T30G | | A57S | A100V | | | | |
| M71 | R28N | L56I | E54K | L99T | 2.12 × 10⁻³ | 117.8 | 1.28 × 10⁻³ | 8.00 |
| | T30G | | A57Y | A100V | | | | |
| M72 | R28N | L56S | E54K | L99T | 3.95 × 10⁻³ | 219.4 | 4.00 × 10⁻⁴ | 2.50 |
| | T30G | | | A100V | | | | |
| M73 | R28N | L56S | E54K | L99T | 3.00 × 10⁻³ | 166.7 | 2.55 × 10⁻⁴ | 1.59 |
| | T30G | | A57N | A100V | | | | |
| | | | S58Y | | | | | |
| | | | E64D | | | | | |
| M74 | R28N | L56S | E54K | L99T | 6.03 × 10⁻³ | 335.0 | 5.97 × 10⁻⁴ | 3.73 |
| | T30G | | A57S | A100V | | | | |
| M75 | R28N | L56S | E54K | L99T | 1.87 × 10⁻² | 1038.9 | 1.16 × 10⁻³ | 7.25 |
| | T30G | | A57V | A100V | | | | |
| M76 | R28N | G50A | A57G | L99T | 1.16 × 10⁻³ | 64.4 | 3.64 × 10⁻⁴ | 2.28 |
| | T30S | L56T | | A100V | | | | |
| M77 | R28N | G50A | E54K | L99T | 0.0143 | 794.4 | 4.77 × 10⁻⁴ | 2.98 |
| | T30S | L56T | A57D | A100V | | | | |
| M78 | R28N | G50A | E54K | L99T | 0.167 | 9277.8 | 1.31 × 10⁻³ | 8.19 |
| | T30S | L56T | A57N | A100V | | | | |
| | | | S58T | | | | | |
| M79 | R28N | G50A | E54K | L99T | 0.19 | 10555.6 | 1.29 × 10⁻³ | 8.06 |
| | T30S | L56T | A57P | A100V | | | | |
| M80 | R28N | L56I | E54K | L99T | 0.0993 | 5516.7 | 2.09 × 10⁻³ | 13.06 |
| | T30S | | A57N | A100V | | | | |
| | | | S58V | | | | | |
| M81 | R28N | L56S | E54K | L99T | 4.29 × 10⁻³ | 238.3 | 4.90 × 10⁻⁴ | 3.06 |
| | T30S | | A57N | A100V | | | | |
| | | | S58E | | | | | |
| M82 | R28N | A51H | A57N | L99T | 6.99 × 10⁻³ | 388.3 | 8.77 × 10⁻⁴ | 5.48 |
| | T30V | L56T | | A100V | | | | |
| M83 | R28N | A51H | E54K | L99T | No | n.d. | 9.33 × 10⁻⁴ | 5.83 |
| | T30V | L56T | A57N | A100V | binding | | | |
| | | | S58M | | | | | |
| | | | H61F | | | | | |
| M84 | R28N | A51H | E54N | L99T | 1.76 × 10⁻² | 977.8 | 1.08 × 10⁻³ | 6.75 |
| | T30V | L56T | A57N | A100V | | | | |

All CDRs including both Kabat and Chothia CDRs. Amino acid residues are numbered sequentially (see FIG. 5). All clones have L3+H1+H3 sequences identical to G1.

K_{D}=k_{off}/kₒₙ. All k_{off} values were determined in a screening mode except those that are underlined, which were obtained by global analysis of a Fab concentration series (G1 was analyzed in a high-resolution mode). Underlined K_{D} values were therefore determined experimentally by measuring kₒₙ. Other kₒₙ values were estimated to be the same as M25.

n.d.=not determined

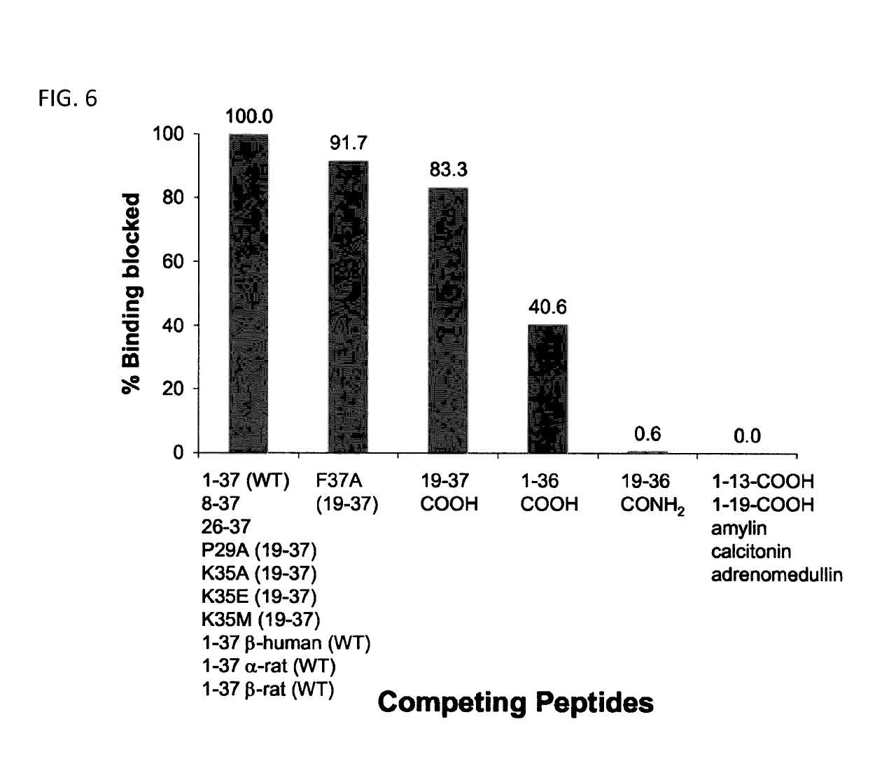

To determine the epitope on human α-CGRP that is recognized by antibody G1, Biacore assays described above were used. Human α-CGRP was purchased as an N-biotinylated version to enable its high-affinity capture via SA sensor chips. The binding of G1 Fab fragment to the human α-CGRP on the chip in the absence or presence of a CGRP peptide was determined. Typically, a 2000:1 mol peptide/Fab solution (e.g., 10 uM peptide in 50 nM G1 Fab) was injected across human α-CGRP on the chip. FIG. 6 shows the percentage of binding blocked by competing peptide. Data shown in FIG. 6 indicate that peptides that block 100% binding of G1 Fab to human α-CGRP are 1-37 (WT), 8-37, 26-37, P29A (19-37), K35A (19-37), K35E (19-37), and K35M (19-37) of human α-CGRP; 1-37 of β-CGRP (WT); 1-37 of rat α-CGRP (WT); and 1-37 of rat β-CGRP (WT). All these peptides are amidated at the C-terminus. Peptides F37A (19-37) and 19-37 (the latter not amidated at the C-terminus) of human α-CGRP also blocked about 80% to 90% of binding of G1 Fab to human α-CGRP. Peptide 1-36 (not amidated at the C-terminus) of human α-CGRP blocked about 40% of binding of G1 Fab to human α-CGRP. Peptide fragment 19-36 (amidated at the C-terminus) of human α-CGRP; peptide fragments 1-13 and 1-19 of human α-CGRP (neither of which are amidated at the C-terminus); and human amylin, calcitonin, and adrenomedullin (all amidated at the C-terminus) did not compete with binding of G1 Fab to human α-CGRP on the chip. These data demonstrate that G1 targets a C-terminal epitope of CGRP and that both the identity of the most terminal residue (F37) and its amidation is important for binding.

Binding affinities of G1 Fab to variants of human α-CGRP (at 37° C.) was also determined. Table 7 below shows the affinities as measured directly by titrating G1 Fab across N-biotinylated human α-CGRP and variants on the chip. Data in Table 7 indicate that antibody G1 binds to a C-terminal epitope with F37 and G33 being the most important residues. G1 does not bind to CGRP when an extra amino acid residue (alanine) is added at the C-terminal (which is amidated).

TABLE 7
Binding affinities of G1 Fab to human α-CGRP and variants measured
at 37° C. (see Table 4 for their amino acid sequences)

| CGRP on chip | kₒₙ (1/Ms) | k_{off} (1/s) | K_{D} (nM) |
|---|---|---|---|
| 1-37 (WT) | 4.68 × 10⁵ | 7.63 × 10⁻⁵ | 0.16 (high |
| | | | resolution K_{D} = 0.06) |
| 19-37 | 4.60 × 10⁵ | 7.30 × 10⁻⁵ | 0.16 |
| 25-37 | 3.10 × 10⁵ | 8.80 × 10⁻⁵ | 0.28 |
| F27A (25-37) | 3.25 × 10⁵ | 1.24 × 10⁻⁴ | 0.38 |
| V28A (25-37) | 3.32 × 10⁵ | 9.38 × 10⁻⁵ | 0.28 |
| P29A (25-37) | 2.26 × 10⁵ | 1.78 × 10⁻⁴ | 0.79 |
| T30A (25-37) | 1.79 × 10⁵ | 8.41 × 10⁻⁵ | 0.47 |
| N31A (25-37) | 2.17 × 10⁵ | 1.14 × 10⁻⁴ | 0.53 |
| V32A (25-37) | 2.02 × 10⁵ | 3.46 × 10⁻⁴ | 1.71 |
| G33A (25-37) | 2.07 × 10⁵ | 0.0291 | 141 |
| S34A (25-37) | 2.51 × 10⁵ | 7.64 × 10⁻⁴ | 3.04 |
| K35A (19-37) | 2.23 × 10⁵ | 2.97 × 10⁻⁴ | 1.33 |
| K35E (19-37) | 5.95 × 10⁴ | 5.79 × 10⁻⁴ | 9.73 |
| K35M (19-37) | 2.63 × 10⁵ | 1.34 × 10⁻⁴ | 0.51 |
| K35Q (19-37) | 1.95 × 10⁵ | 2.70 × 10⁻⁴ | 1.38 |
| F37A (25-37) | 8.90 × 10⁴ | 8.48 × 10⁻³ | 95 (solution |
| | | | K_{D} = 172 nM) |
| 38A (25-38A) | | — | No binding detected |

The above data indicate that the epitope that antibody G1 binds is on the C-terminal end of human α-CGRP, and amino acids 33 and 37 on human α-CGRP are important for binding of antibody G1. Also, the amidation of residue F37 is important for binding.

### Example 5

### Effects of Anti-CGRP Antagonist Antibody and TRPVs Mutation on Metabolic Health and Longevity

This example describes methods used to determine if reduced pain perception could positively regulate mammalian aging by targeting evolutionary conserved TRPVs. The mechanism of this increased longevity is also explored.

Longevity Study on Mice

TRPV1 mutant mice were created and contain a deletion of an exon encoding part of the fifth and all of the sixth putative transmembrane domain and the pore-loop region of the TRPV1 gene. Homozygous animals are viable, fertile and appear normal. The longevity study was generated from 16 breeders pairs of homozygous TRPV1 mutant backcrossed eleven times to C57BL/6J and wild-type (WT) C57BL/6J breeder pairs both obtained from the Jackson laboratory (Bar Harbor, Me.). Animals used from broods of similar size were used for each lifespan study. TRPV1 genotyping was used to assign both experimental and control groups. Post mortem genome scan analysis was conducted to confirm that the genetic background was 100% identical among genotypes (Table 11).

Mice were housed in groups of 2 to 5 same-sex littermates under pathogen-free conditions, with ad libitum access to water and chow (PicoLab Rodent 20 5053*, LabDiet). Individuals were monitored daily and weighed monthly, but were otherwise left undisturbed until they died. Survival was assessed from 90 females (48 TRPV1 KO, 42 WT) and 70 males (38 TRPV1 KO, 32 WT) mice, with 100% of animals dead by the time of this report. Shortly after natural death, mice were immersed in formalin, dried in 70% Ethanol. Tissues were dissected, embedded in paraffin and stained by Hematoxylin and Eosin. Postmortem pathological analysis was performed by the veterinary staff. An additional experimental cohort was created to perform metabolic studies and generate primary neuronal cultures described below. Kaplan-Meier survival curves were constructed using known birth and death dates, and differences between groups were evaluated using the log-rank test.

Metabolic Study on Mice

Indirect calorimetric studies were conducted in a Comprehensive Lab Animal Monitoring System (Columbus Instruments). For GTT and GSIS, glucose (2 g/kg weight) was intraperitoneally administered and blood glucose was measured from tail bleeds at indicated times after injection using a One Touch Ultra glucometer (LifeScan). For the ITT, 5 hour fasted mice were injected with 1 u/kg of human insulin (Humulin; Eli Lily) and glucose was measured as in the GTT. Insulin-stimulated signaling in liver and muscle tissue was performed as previously described. Osmotic pumps (Alzet) diffusing CGRP8-37 (Tocris Bioscience) at 5.5 mg/mL were implanted in 22 month old C57BL/6J males (National Institute of Aging). CGRP8-37 is a truncated version of anti-CGRP antagonist antibody. Serum insulin was measured using an UltraSensitive Insulin ELISA kit (Crystal Chem). Serum levels of fasted leptin, adiponectin were measured using Quantikine ELISA Kits (R&D systems). Triglycerides and cholesterol were measured using Thermo enzymatic colorimetric methods, following manufacturer's instructions. CGRP was measured using ELISA (EIAab).

Quantitative PCR Analysis and Protein Analysis

Pancreatic islets were isolated. 25-40 DRGs were excised from each mouse and homogenized in trizol. RNA was isolated using trizol/chloroform extraction and RNEasy Qiagen columns. RNA was converted into cDNA using quantitect reverse-transcription kit. Gene expression was assessed by qPCR using SYBR green. Primers sequences are listed in Table 8.

Immunohistochemistry and Histological Studies

For immunohistochemistry, tissues were immersed in 4% paraformaldehyde in PBS for 4-16 hours at 40 C followed by cryoprotection in 30% sucrose in PBS overnight. Tissues were frozen in OCT (Tissue-Tek) and sectioned on a cryostat. 12 μm thick sections were immunoblotted with primary antibodies in PBS containing 2% Donkey serum and 0.4% Triton x100. Antibodies used were anti-CRTC1 (Bethyl laboratories) at 1:100, anti-CRTC1 (LS Bio) at 1:500, anti-CGRP (Bachem) at 1:500, anti-TRPV1 (Millipore) at 1:500, anti-insulin (Millipore) at 1:500, anti-glucagon (Millipore) at 1:7000, secondary antibodies (Alexxa) were diltuted at 1:600. Sections were imaged using a LSM 710 confocal microscope. For histological analysis, tissues were immersed in formalin overnight, dried in 70% Ethanol and embedded in paraffin. To measure β-cell mass, whole pancreata were weighed and sectioned, prior to insulin-immunostaining and labeling with the Envision+DAB sytem peroxidase (Dako) with a rabbit anti-insulin antibody (Cell Signaling) at 1:100 and counterstaining with hematoxylin. Slides were imaged with a VS120 slide scanner (Olympus) and β-cell mass was measured by point-counting morphometry using the VS-ASW software.

Cell Culture Studies

MIN6 insulinoma cells were obtained at passage 18 from Ulupi Jhala (UCSD, La Jolla, Calif.) and were cultured in DMEM (Life technologies) containing 11 mM glucose, 10% FBS, Glutamax (Invitrogen) and 10 μM β-mercapthoethanol as previously described (Huising et al., Proc. Natl. Acad. Sci. 107:912-917, 2010). Glucose stimulation was performed at low (2.8 mM) and high (16.8 mM). Treatments were diluted in Krebs-Ringer bicarbonate buffer with 2.8 mM glucose.

DRG neurons were dissociated from mice and dissociated by trituration in papain after collagenase treatment and seeded on a monolayer of cortical astrocytes in chamber slides (Fisher Scientific) coated with Geltrex (Life technologies) and cultured in DMEM/F12 supplemented with B27, N2, 50 ng/ml rat BDNF, 50 ng/ml rat β-NGF and postmitotic inhibitors 17.5 μg/ml uridine and 7.5 μg/ml 5-fluoro-2-deoxyurine. To allow for axonal regrowth and formation of synapses among DRG neurons, neurons were cultured for 7-10 days on a monolayer of rat cortical astrocytes.

Neurons were pretreated 4 hours with TTX (1 μM) prior either vehicle control or antagonist blockade (2 hours) followed by 1 hour drug stimulation supplemented with Leptomycin B (10 ng/ml) which inhibits nuclear export to detect CRTC1 nuclear accumulation. Cap, FSK and Leptomycin B were obtained from Sigma-Aldrich, SB366791 and TTX from Tocris Biosciences and CsA from Calbiochem. Drugs were applied at the following concentrations: Cap (1 μM), FSK (25 μM), KCl (30 mM), SB366791 (2 μM) and CsA (10 μM). Neurons were then fixed in 4% paraformaldehyde in PBS and prepared for immunohistochemistry.

C. elegans Experiments

osm-9(ky4), ocr-2(ak47) and N2 strains were obtained from the Caenorhabditis Genetics Center. AGD418 (N2; uthIs205 [Pcrtc-1::crtc-1::RFP::unc-54 3′UTR, rol-6]) and AGD466 (N2, uthEX222 [crtc-lp::crtc-1 cDNA (576A, 5179A)::tdTOMATO::unc-54 3′UTR; rol-6(su1006)]) were generated elsewhere (Mair et al., 2011). osm-9(ky4), ocr-2(ak47) strains were outcrossed 6 times to N2. AGD418 and AGD466 were crossed with osm-9(ky4); ocr-2(ak47). Lifespan analyses were performed at 20° C. and were repeated 2 to 4 times. 100 to 120 animals were used per condition and scored every second day. Lifespans were performed on HT115 with control GFP RNAi or tax-6 RNAi as indicated. GraphPad Prism 5 was used for statistical analysis. p values were calculated using the log-rank (Mantel-Cox) method.

Results: Mice Lacking TRPV1 are Youthful and Long-Lived

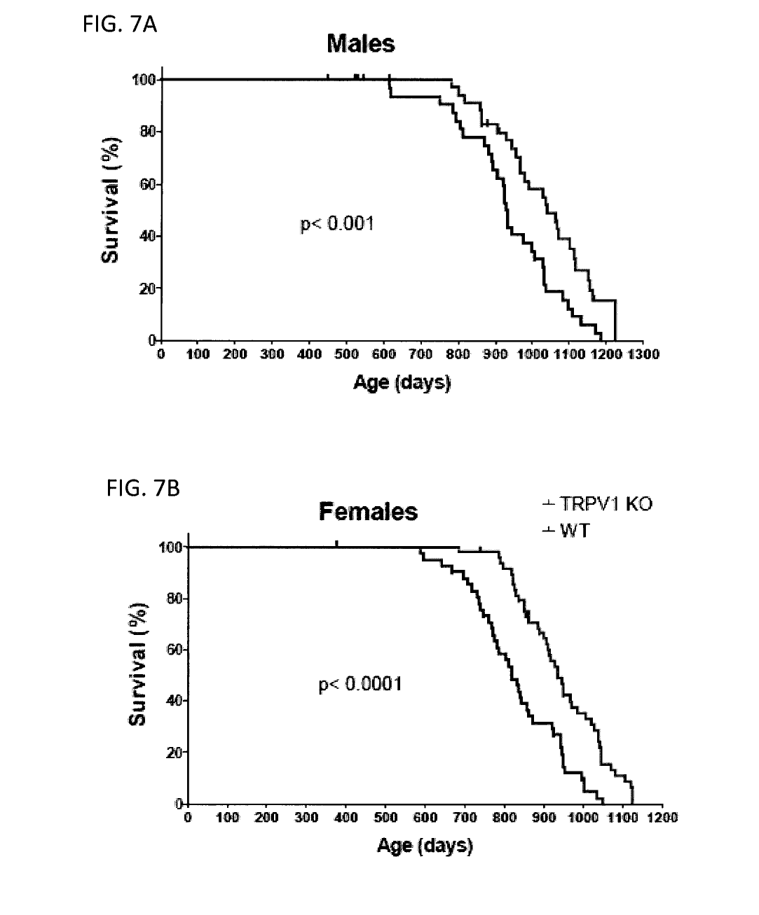

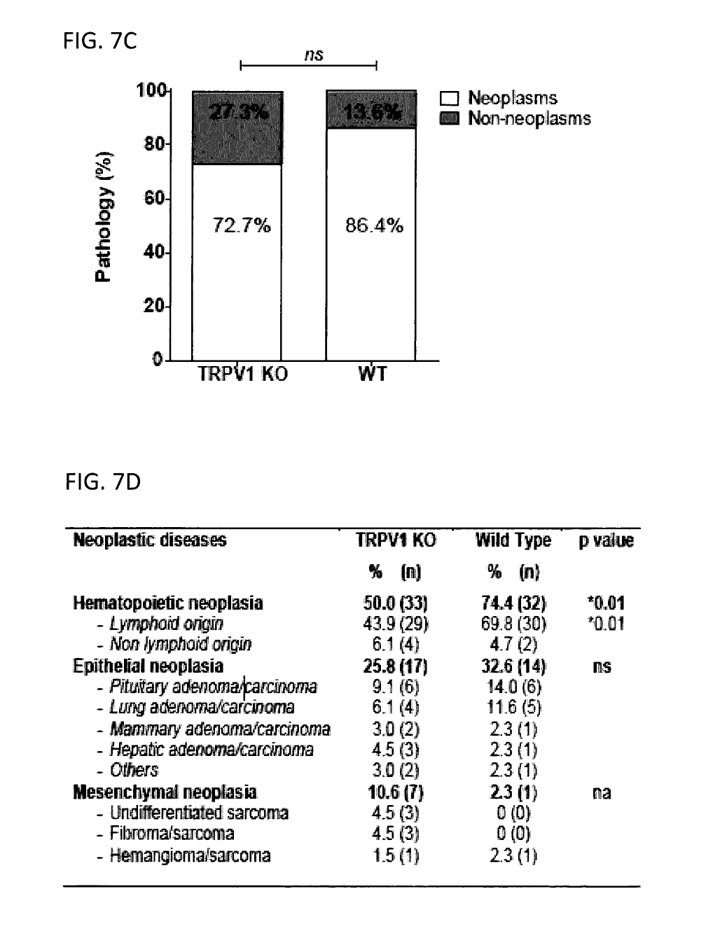

Animals lacking TRPV1 receptors were exceptionally long-lived as indicated by their Kaplan-Meier survival curves, median, and maximal lifespan (FIGS. 7A, 7B and Table 9). Under normal fed ad libitum conditions, the TRPV1 mutation was not sex-specific in its effects: longevity in both genders was extended to a similar extent, with 11.9% increase in male TRPV1 mutants and 15.9% increase in median female lifespan compared to wild-type, isogenic C57BL/6 controls (WT). On average, TRPV1 mutant males lived almost 100 days and TRPV1 mutant females lived 130 days longer than control animals (mean lifespan, Table 9). Maximum lifespan was strongly extended in female mutant mice.

Cancer incidence was determined by conducting post-mortem necropsy and histopathology (FIGS. 7C, 7D). The prevalence of cancer tended to be reduced in TRPV1 mutant versus control mice (FIG. 7C). Neoplasms, which are the primary cause of death observed in our mouse cohort, comprised a wide panel of cancer types (FIG. 7D). Notably, the incidence of hematopoietic neoplasia of lymphoid origin, the most prevalent cancer observed in both genotypes, was reduced in TRPV1 mutant animals. Other cancer types including sarcomas, carcinomas, pituitary and lung adenomas were also present in both genotypes, however the small number of observations did not allow for any conclusion regarding the incidence of these pathologies among genotypes. Furthermore, no significant differences were observed in the incidence of non-neoplastic diseases between genotypes (FIG. 7C and Table 10).

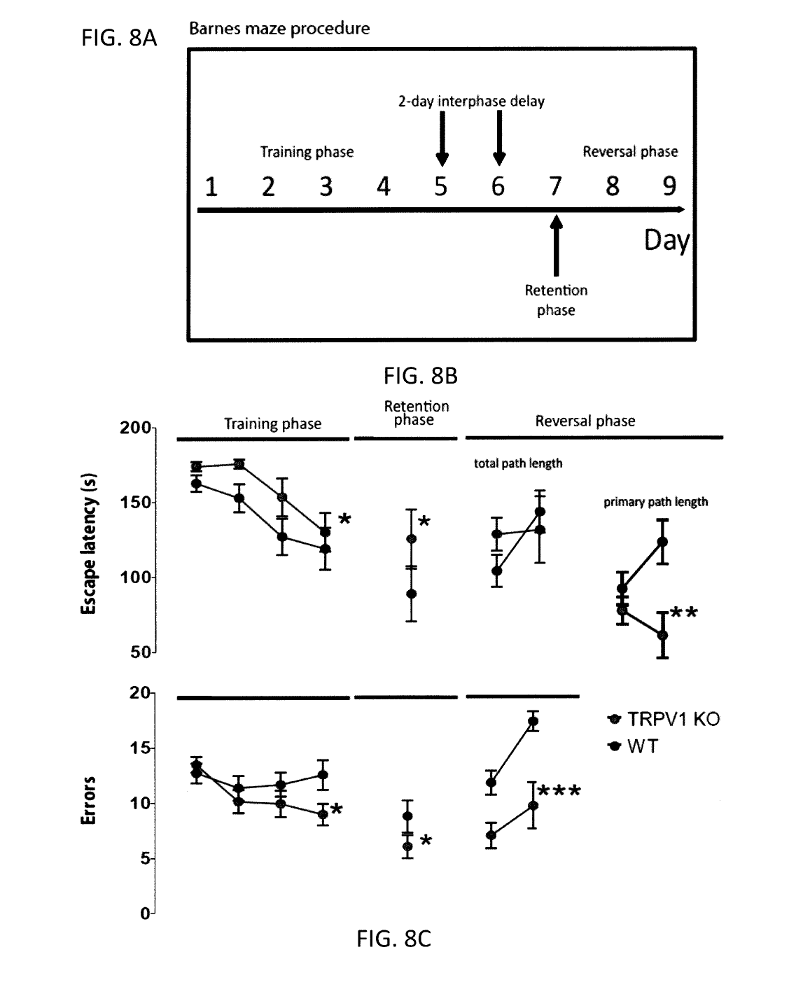

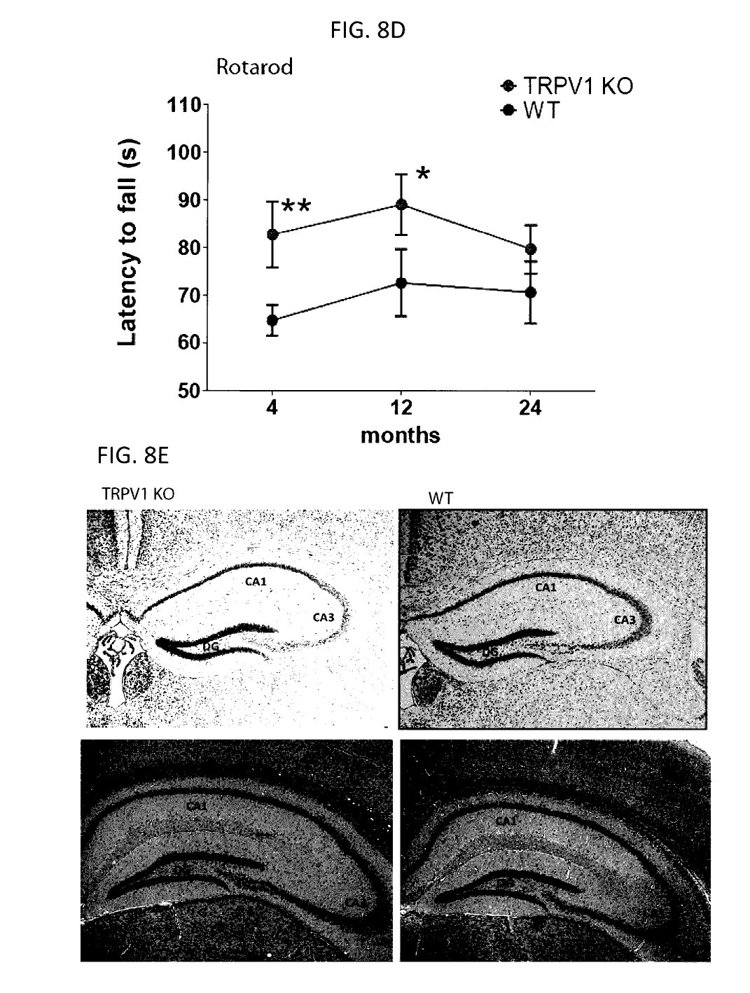

Consistent with improved longevity and reduced aging, 30 month-old TRPV1 mutant mice displayed improved spatial memory compared to their age-matched control animals when challenged with a training, retention and reversal paradigm in a Barnes maze test (FIGS. 8A, 8B, 8C). Motor coordination, measured by the latency to fall from a rotating rod, was also delayed in the long-lived TRPV1 mutant mice (FIG. 8D). Because the presence of TRPV1 has been detected in the hippocampus (Mezey et al., Proc. Natl. Acad. Sci. 97, 3655-3660, 2000; Sasamura et al., Neuroreport 9, 2045-2048, 1998), the neuronal density in the CA1, CA3 and dentate gyrus (DG) areas of the hippocampus was examined by Nissl staining (FIG. 8E). The distribution of Nissl-positive neurons in these areas was similar in WT and TRPV1 mutant mice, suggesting that improved cognitive function in the TRPV1 mutants is not due to increased neuronal numbers in the TRPV1 mutant animals, but rather to a global improved health-span in these mice.

Results: Long-Lived TRPV1 Mutant Mice do not have Reduced Growth Rates or Altered IGF-1 Levels

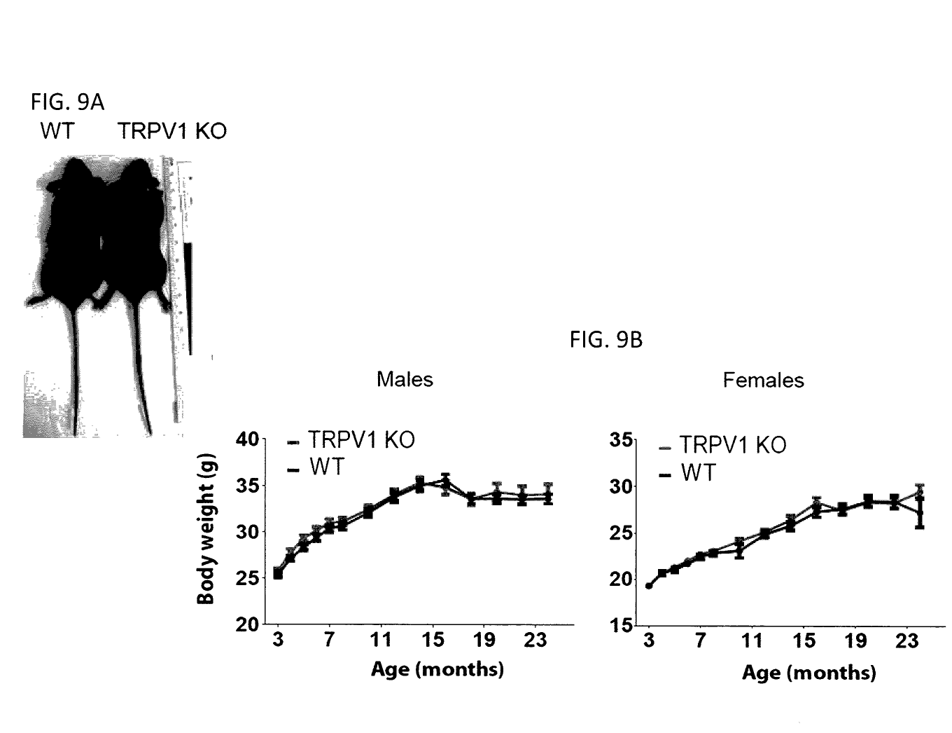

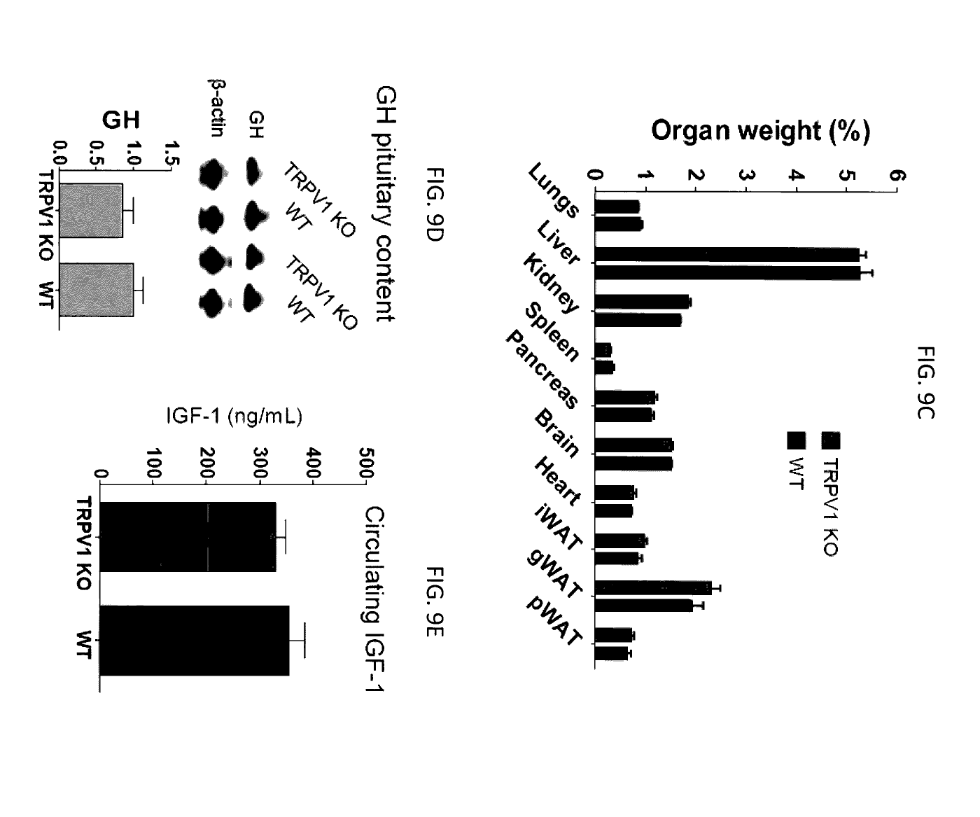

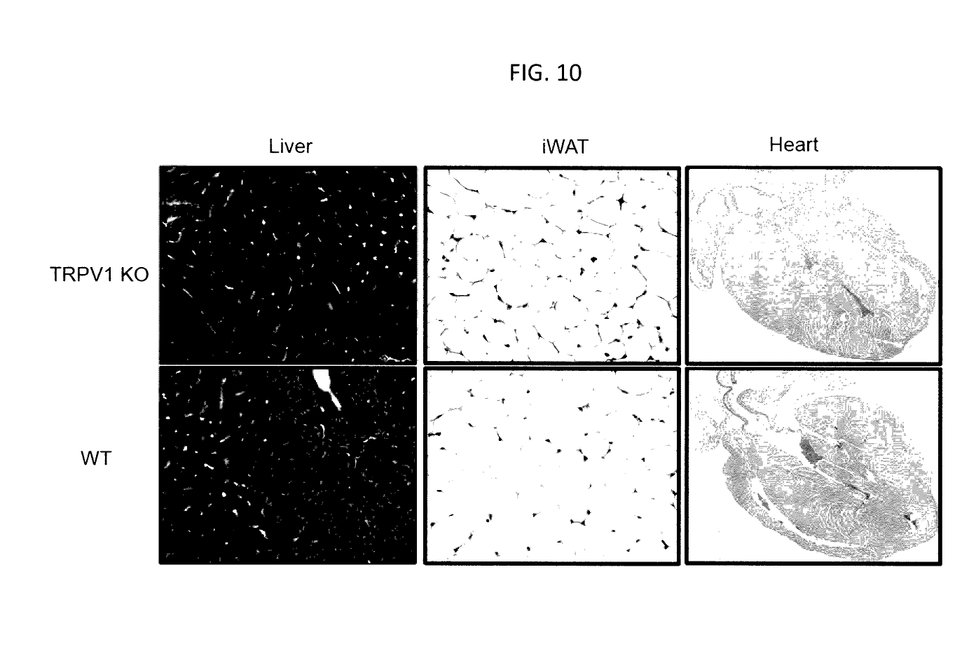

Reduced growth hormone (GH) and/or insulin growth factor (IGF-1) signaling have been tightly linked to longevity, often resulting in delayed growth and small adult animals (Bluher, Science 299:572-574, 2003; Ortega-Molina et al., Cell Metab. 15:382-394, 2012; Selman et al., FASEB J. 22:807-818, 2008). Unlike GH/IGF-1 mice, TRPV1 mutant mice appeared similar in size to WT mice (FIG. 9A). Their growth curves were identical to WT controls in both genders throughout aging (FIG. 9B), in contrast to mouse models with altered activity in either growth hormone (GH) or IGF-1 signaling (Bluher, Science 299:572-574, 2003; Brown-Borg et al., Nature 384:33, 1996; Coschigano et al., Endocrinology 141:2608-2613, 2000, Coschigano et al., Endocrinology 144:3799-3810, 2003; Flurkey et al., Proc. Natl. Acad. Sci. 98:6736-6741, 2001, Flurkey et al., Mech. Ageing Dev. 123:121-130, 2002; Holzenberger et al., Nature 421:182-187, 2003; Ortega-Molina et al., Cell Metab. 15:382-394, 2012; Selman et al., FASEB J. 22:807-818, 2008, Selman et al., Science 326:140-144, 2009; Zhang et al., eLife 1, 2012). Analysis of body composition of age and gender matched controls confirmed that organs including lung, liver, kidney, spleen, pancreas, brain, heart and different fat pads (inguinal, gonadal, retroperitoneal) were the same size in both genotypes (FIG. 9C). Basic histological analysis by H&E staining of key metabolic tissues (liver, white fat, heart) did not reveal morphological differences between the wild-type and TRPV1 mutant mice (FIG. 10). Consistent with a normal body weight, GH content in the pituitary was normal in the TRPV1 mutant adult mice (FIG. 9D). In addition, plasma levels of IGF-1, which is regulated by GH, were identical in both genotypes (FIG. 9E). Therefore loss of TRPV1 appears to modulate the aging process in a mechanism independent of the GH/IGF-1 axis.

Results: Long-Lived TRPV1 Mutant Mice have a Youthful Metabolic Profile

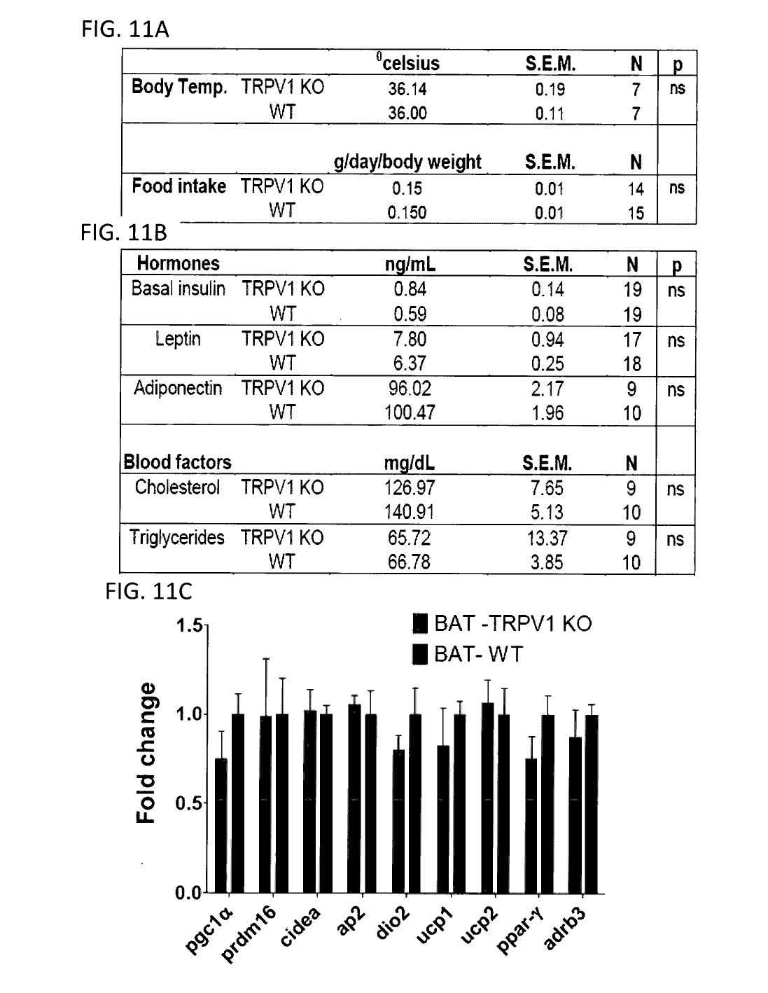

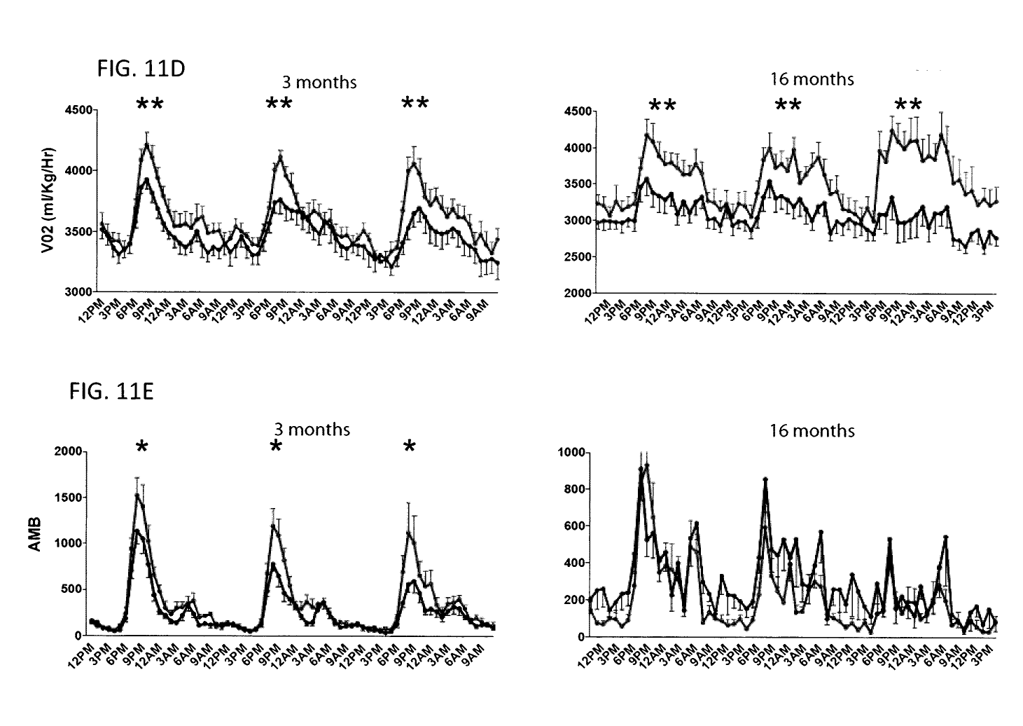

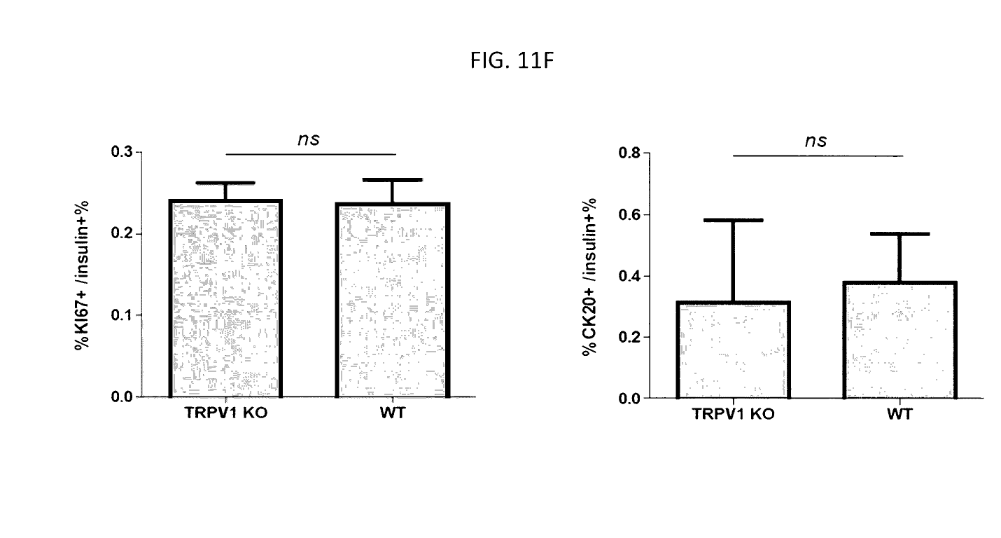

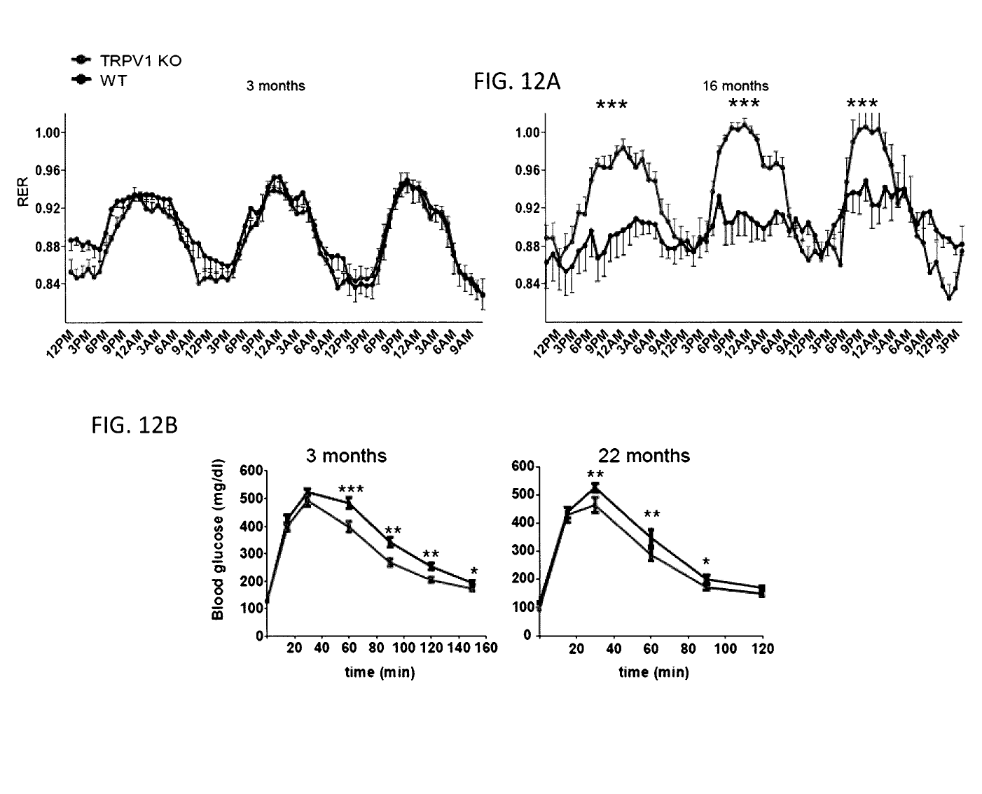

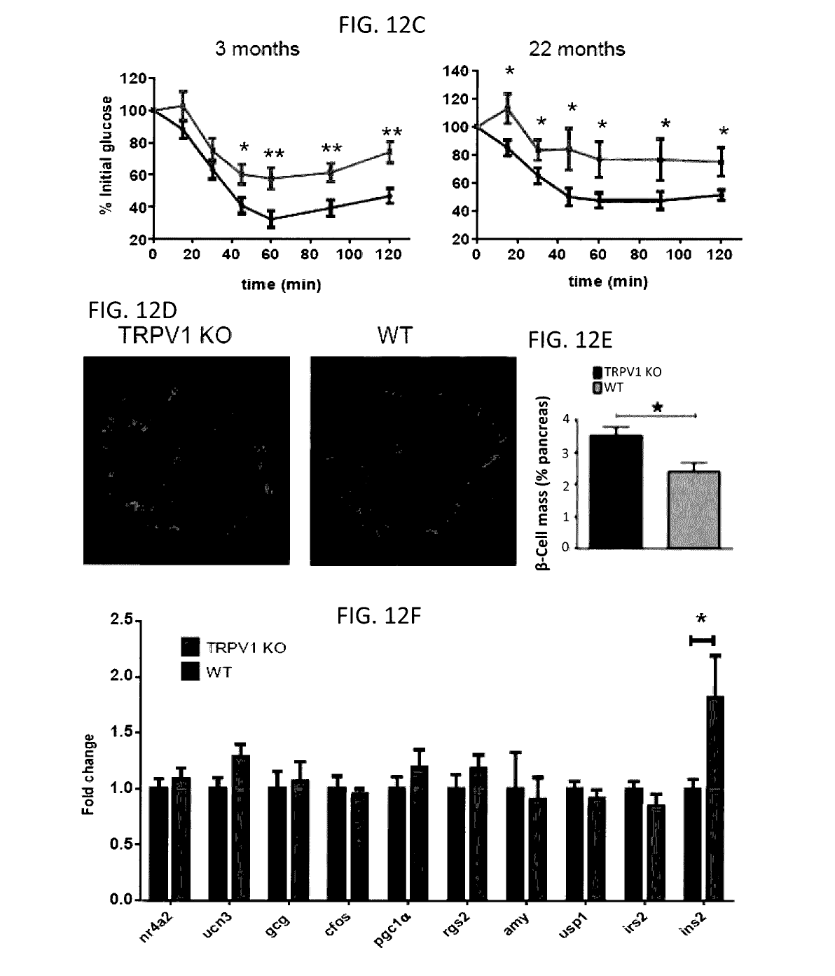

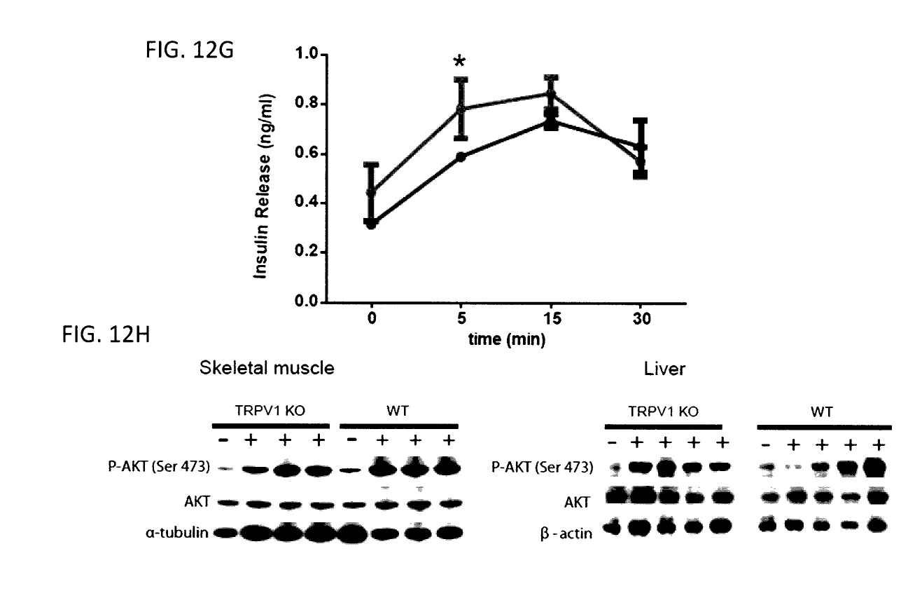

Altered metabolic programs have been associated with many longevity pathways ranging from worms to mice. It was determined whether the TRPV1 mutant mice had a distinct metabolic profile in comparison to wild-type animals. Analysis of body temperature, indicative of metabolic activity and reported to be low in dietary restricted and long-lived Ames dwarf mice (Brown-Borg et al., Nature 384:33, 1996), was unaffected in age-matched TRPV1 mutant mice compared to control animals (FIG. 11A). Similarly, food intake, which can influence lifespan through dietary restriction, was unaffected (FIG. 11A). A general assessment of blood factors indicative of metabolic activity revealed no difference in fasting insulin levels, leptin, adiponectin or circulating triglycerides and cholesterol across genotypes (FIG. 11B). There were no alterations in the expression of brown-fat-specific genes characteristic of thermogenesis and brown fat development in the interscapular brown adipose tissue (FIG. 11C). However, TRPV1 mutant animals retained a youthful metabolic program compared to age-matched control mice (FIG. 12A). In particular, the respiratory exchange ratio (RER), which measures the daily transition from glucose to lipid metabolism, retained a youthful circadian shift from night to day in old TRPV1 mutant animals. At 3 months, both WT and TRPV1 mutant mice displayed identical circadian oscillations from night to day. However at late age, the TRPV1 mutants retained circadian transitions of similar order of magnitude compared to that of young mice, whereas WT animals lost their circadian shift. In fact, decline in energy expenditure involving a lower RER is commonly observed in old mice indicating that aging mice have a proportional substrate preference towards fat (Houtkooper et al., Sci. Rep. 1, 2011). It was observed that the TRPV1 mutant mice do not have this preference in late life.

Concurrent with improved metabolism, oxygen consumption was enhanced in TRPV1 mutants at both young and old time points compared to age-matched WT controls at the peak of nocturnal activity (FIG. 11D). TRPV1 mutants maintained their VO₂ maximum consumption late in life, in contrast to WT animals that display a decline in maximum VO₂ consumed with aging. Interestingly, voluntary activity was slightly higher in young TRPV1 mutant animals but diminished to the same extent as WT when old (FIG. 11E). Taken together, these data indicate that the youthful energy expenditure observed in old TRPV1 mutant animals is not due to increased voluntary exercise, but appears to be due to more efficient maintenance of metabolism with age.

Result: Long-Lived TRPV1 Mutant Mice have Altered Insulin Metabolism

To uncover the mechanism underlying the youthful metabolic phenotype of the TRPV1 mutant mice, the glucose metabolic profile of these mice was characterized. It was hypothesized that TRPV1 mutant animals should be glucose tolerant. It was observed that TRPV1 mutant mice were markedly more glucose tolerant than WT at 3 months and 22 months of age, which correspond respectively to “young” and “old” time points in a Glucose Tolerance Test (GTT, FIG. 12B). Fasted glucose levels were unchanged between mutant and wild-type mice. As improved glucose tolerance is often associated with increased insulin sensitivity, how efficiently TRPV1 mutant mice were at clearing blood glucose after insulin injection through an Insulin Tolerance Test was analyzed (ITT, FIG. 12C). Despite improved glucose tolerance, it was observed that blood glucose levels from young TRPV1 mutant mice decreased by ˜40% following acute insulin challenge, contrary to WT controls which dropped by 67%. At 22 months, WT mice displayed the expected age-onset insulin resistance as their drop in blood glucose was 14% less efficient than young WT mice.

The insulin resistance of the TRPV1 mutant was also further aggravated in older animals by 19%. As insulin resistance is a hallmark of type 2 diabetes, leading to β-cell failure, we analyzed pancreatic β-cell islets morphology in adult mice. No difference was observed in islet architecture when staining for insulin and glucagon in islets from mutant or wild-type mice, suggesting that TRPV1 mutant islets are normal (FIG. 12D). Further investigation revealed that β-cell mass was increased in the TRPV1 mutant mice, suggesting that β-cell survival was improved in these mice independently of their proliferation or neogenesis rate (FIGS. 12E and 11F). Gene expression of isolated islet mRNA through quantitative real-time PCR (qPCR) did not reveal transcriptional differences between TRPV1 mutants and WT islet endocrine markers (FIG. 12F). Levels of glucagon (gcg), urocortin-3 (ucn3), amylin (amy), insulin-receptor substrate 2 (irs2), the nuclear hormone receptor nr4a2, the fos gene cFos, the ubiquitin carboxyl-terminal hydrolase 1 (usp1), the regulator of G-protein signaling 2 (rgs2) and Peroxisome proliferator-activated receptor gamma coactivator 1-alpha (pgc1α) were unchanged across genotypes.

Because of the increased β-cell mass observed in the TRPV1 mutant mice, we assumed that the improved glucose tolerance was achieved through insulin hyper-secretion in response to glucose in order to compensate for their mild insulin resistance. Consistent with this hypothesis, either chemo-denervation of TRPV1 neurons or chemical inhibition of TRPV1 in rodents increases glucose-dependent insulin secretion (Gram et al., Eur. J. Neurosci. 25:213-223, 2007; Tanaka et al., Life Sci. 88:559-563, 2011). Indeed qPCR analysis of pancreatic islet mRNA revealed that the insulin gene (ins2) was ˜2 fold upregulated in the TRPV1 mutant cells. To directly assess the link between TRPV1 and insulin secretion, a glucose stimulated insulin secretion experiment (GSIS) was performed where insulin levels are monitored upon glucose injection (FIG. 12G). We found that insulin release was significantly more pronounced in TRPV1 mutant mice than WT (p<0.05), thus explaining the improved glucose tolerance of TRPV1 mutants. Consistent with TRPV1 mutant mice being healthy and not harboring symptoms of type 2 diabetes, fasted insulin levels were similarly low as compared to wild-type mice (FIGS. 12G, 11B). Therefore, the TRPV1 mutant mice are not hyper-insulinemic, but rather display an elevation in prandial insulin to respond more robustly to glucose challenge. To further investigate the nature of the insulin resistance of the TRPV1 mutant mice, we evaluated insulin-stimulated signaling in liver and skeletal muscle by analyzing AKT phosphorylation, which constitutes a downstream effector of the insulin receptor pathway. Upon intravenous insulin injection, phosphorylation of serine 473 of the serine/threonine kinase AKT protein was unaltered in both tissues of the TRPV1 mutant animals compared to control animals (FIG. 12H), indicating that insulin signaling is functional in TRPV1 mutants in response to insulin.

Taken together, loss of TRPV1 results in long-lived mice that are not growth retarded and do not have altered IGF-1 levels or responsiveness, yet have a youthful, metabolic profile with increased glucose tolerance mediated by increased insulin secretion upon glucose challenge. These data suggest that impaired TRPV1 sensory neuron function has beneficial effects on glucose homeostasis and longevity. Intrigued by the ability of these mice to live long by a mechanism not canonical to established longevity paradigms (GH/IGF-1 and dietary restriction), the mechanism by which TRPV1 neurons could modulate this pro-longevity signal was investigated.

Result: C. elegans TRPV Regulates Lifespan Through CRTC1/CREB

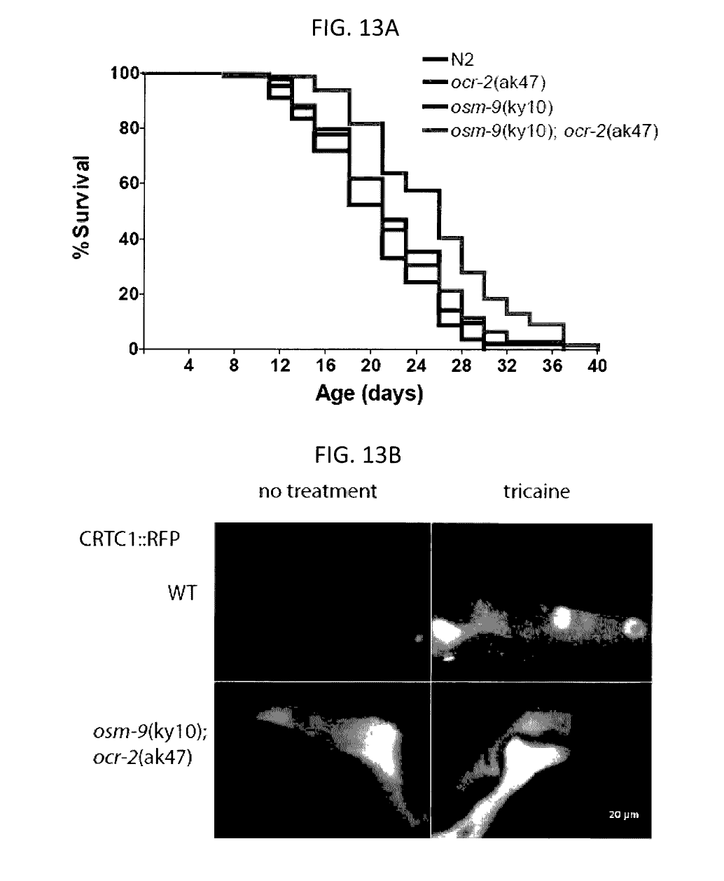

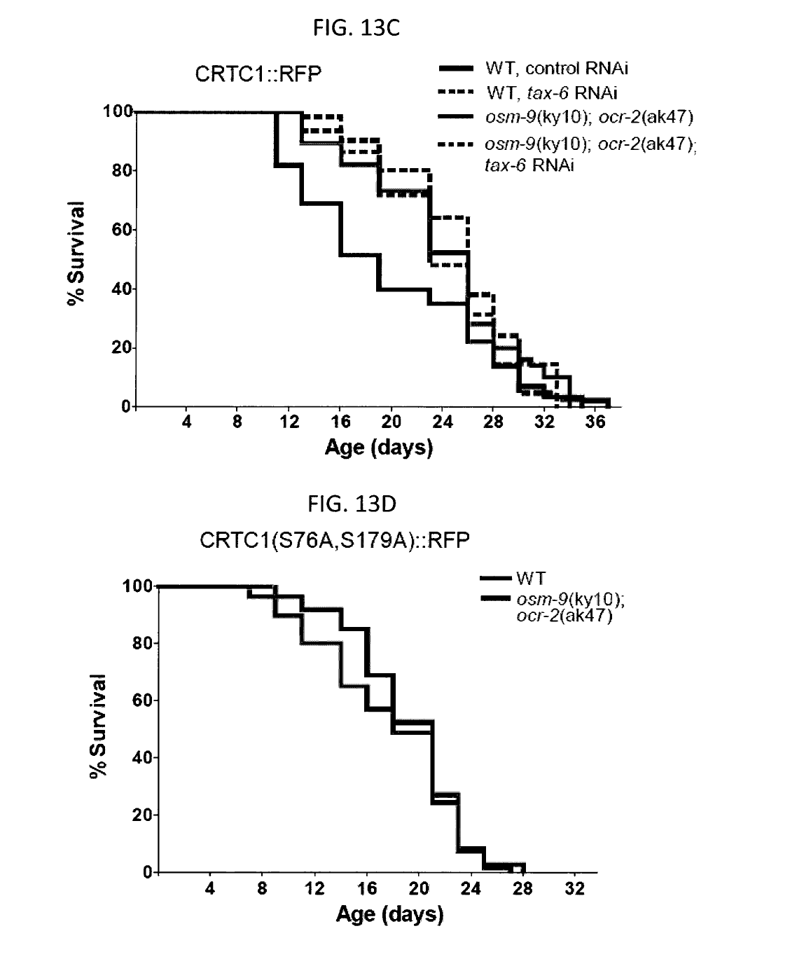

Confronted with the complexity of the mammalian neuroendocrine system and its impact upon metabolism, the nematode C. elegans was used to reveal the possible mechanism by which TRPV1 could modulate metabolism and the aging process. Despite a higher level of integration and compensation, the functional organization of the mammalian chemosensory organs displays many striking similarities to worms at both anatomical and signaling levels. Conserved signaling molecules include members of the TRPV channel family. In worms, osm-9 and ocr-2 function as a channel complex that transduces signals from olfactory, nociceptive and serotonergic neurons (Colbert et al., J. Neurosci. Off. J. Soc. Neurosci. 17:8259-8269, 1997; Tobin et al., Neuron 35:307-318, 2002; Zhang, Development 131:1629-1638, 2004). Much like the mouse, loss of TRPV in the worm resulted in increased longevity (FIG. 13A, Table 11). Using null mutants of both osm-9(ky4) and ocr-2(ak47), we observed that loss of either trpv resulted in a modest increase in longevity, consistent with the functional redundancy of this receptor pair (Colbert et al., J. Neurosci. Off. J. Soc. Neurosci. 17:8259-8269, 1997; Tobin et al., Neuron 35:307-318, 2002; Zhang, Development 131:1629-1638, 2004). However, loss of both osm-9 and ocr-2 resulted in a robust extension of lifespan up to 32% compared to control animals.

In response to ligands, TRPV receptors transduce signals to the cytoplasm of affected cells through intracellular calcium increase (Venkatachalam and Montell, Neurobiol. Aging 14:287-293, 2007). One of the major transponders of calcium flux in the cell is calcineurin (Mellstrom et al., Physiol. Rev.88:421-449, 2008). The worm calcineurin ortholog, the calcium-activated calcineurin catalytic A subunit, tax-6, plays an intricate role in the aging process (Dong et al., Science 317:660-663, 2007; Mair et al., Nature 470:404-408, 2011). Loss of tax-6 results in long-lived animals and hyperactivation results in short lifespan. One essential target of tax-6 to regulate the aging process in worms is the highly conserved crtc-1 (CREB-Regulated Transcriptional Coactivator 1). Dephosphorylation of CRTC1 on serines 76 and 179 by tax-6 results in nuclear localization, modulation of CREB transcriptional targets and reduced longevity. Opposing calcineurin, AMPK monitors energy sources and phosphorylates CRTC1, retaining CRTC1 in the cytoplasm. Consistent with loss of tax-6 resulting in increased longevity, increased activity of AMPK results in increased longevity through phosphorylation of CRTC1 at serines 76 and 179, sites counteracted by tax-6 (Mair et al., Nature 470:404-408, 2011). While the circuitry downstream of calcineurin to regulate longevity is well established, upstream mediators of this pathway are unknown.

Because of the link between longevity and calcium regulation, we asked if tax-6 and crtc-1 could function downstream of trpv-mediated longevity. Upon tricaine treatment, a drug that increases intracellular calcium in cells, CRTC1 shuttles to the nucleus in wild-type animals, but remains strictly cytoplasmic in tax-6(ok2065) mutants (Mair et al., Nature 470:404-408, 2011). Similarly to tax-6 mutant worms, trpv mutants (osm-9; ocr-2 double mutant animals) retained cytoplasmic localization of CRTC1 upon tricaine treatment (FIG. 13B), indicating that TRPV functions upstream within the tax-6/crtc pathway. Consistent with TRPV functioning upstream in the calcineurin pathway, the increased longevity caused by loss of trpv in the worm was completely dependent upon the CRTC1 longevity pathway. Inactivating tax-6, which extends lifespan in wild-type animals, did not further increase the lifespan of the trpv mutants (FIG. 13C). Concordant with tax-6 modulating longevity through post translational modifications of CTRC1, we found that the increased longevity of the trpv mutants was abrogated when crtc-1 was mutated at the calcineurin dephosphorylation sites S76A, S179A, making it constitutively nuclear (FIG. 13D). Therefore the lifespan extension caused by loss of trpv signaling was completely dependent on nuclear exclusion of CRTC1 at the same phosphorylation sites used for regulation by AMPK and calcineurin. Taken together, these results indicate that a subset of chemosensory neurons utilizes a TRPV calcium signaling cascade to adjust the worm metabolism with environmental conditions by modulating CRTC1/CREB activity that ultimately dictates longevity of the animal.

Result: TRPV1 Regulates CRTC1/CREB in Mice

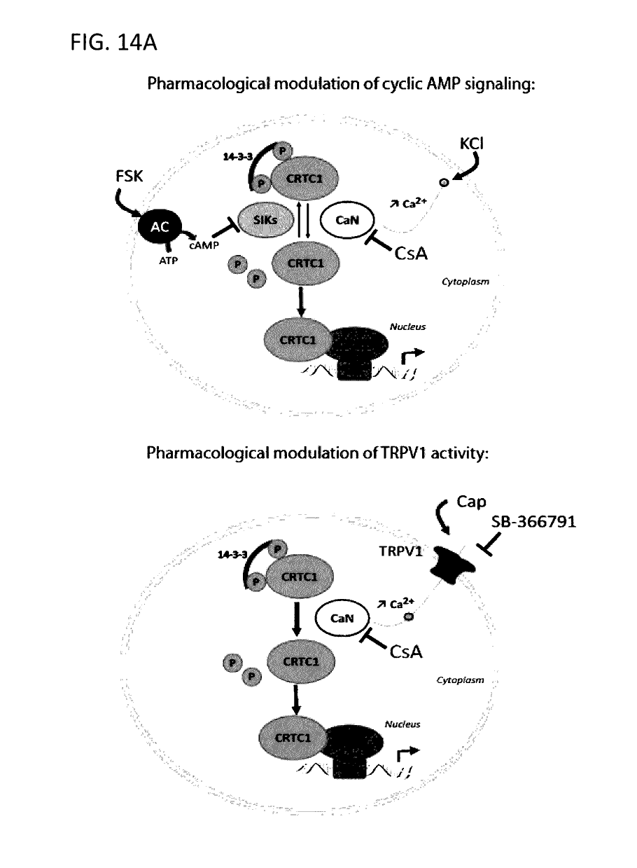

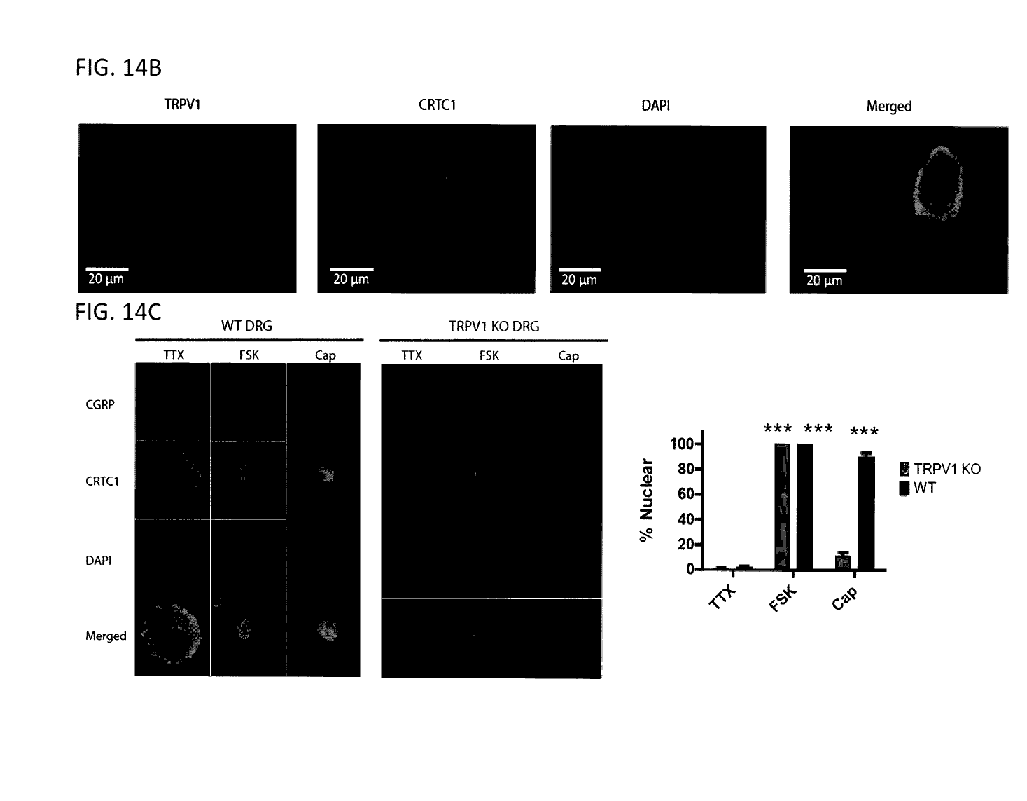

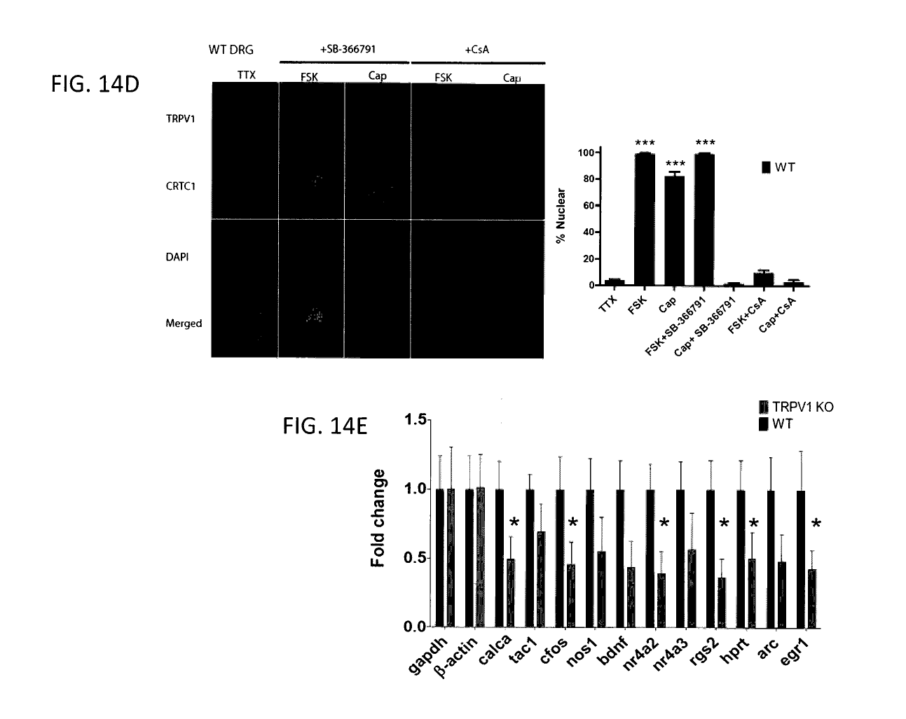

It was determined whether the same system might be engaged in the long-lived TRPV1 mutant mice. The cellular distribution of the most abundant CREB-regulated transcriptional coactivator was examined in neuronal tissues, CRTC1, in dissociated dorsal root ganglion (DRG) neuronal cultures from TRPV1 mutant and wild-type mice (FIGS. 14A-14E). Previous findings indicate that in hippocampal cultures, CRTC1 shuttles to the nucleus of excitatory cells under glutamatergic synaptic activity using a calcium/calcineurin cascade (Ch'ng et al., Cell 150:207-221, 2012; Kovács et al., Proc. Natl. Acad. Sci. 104:4700-4705, 2007). The persistence of the nuclear localization of CRTC1 is regulated by cAMP levels. To evaluate whether calcium and cAMP cascades regulate dynamic shuttling of CRTC1 in DRG neurons, the nuclear to cytoplasmic trafficking of CRTC1 was analyzed in the cell bodies of DRG neurons isolated from TRPV1 mutants by pharmacologically manipulating these pathways (FIG. 14A). Immunoreactivity either directly against TRPV1 or the TRPV1 co-marker CGRP allowed for recognition of small to medium-size nociceptor neurons expressing TRPV1 (Bernardini et al., Neuroscience 126:585-590, 2004; Caterina et al., Nature 389:816-824, 1997; Price and Flores, J. Pain Off. J. Am. Pain Soc. 8:263-272, 2007; Szallasi et al., Brain Res. 703:175-183, 1995). CRTC1 immunoreactivity was observed in peripheral DRG neurons, with a cellular distribution similar to hippocampal cultures (Ch'ng et al., Cell 150:207-221, 2012; Kovács et al., Proc. Natl. Acad. Sci. 104:4700-4705, 2007). Indeed, under basal conditions, CRTC1 localizes to axons, dendrites and the soma of the primary neurons (FIG. 14B). The sodium channel blocker Tetrodotoxin (TTX) was used to reduce endogenous synaptic activity and prevent calcium entry through NMDA receptors.

Under TTX pretreatment, the distribution of CRTC1 remained mostly cytoplasmic as observed in basal conditions (FIG. 14C). To establish conditions to monitor CRTC1 shuttling, stimulation of the neurons with a combination of Forskolin (FSK), which raises cAMP levels therefore decreasing CRTC phosphorylation (Kovács et al., Proc. Natl. Acad. Sci. 104:4700-4705, 2007; Screaton et al., Cell 119:61-74, 2004), and KCl, which triggers calcium entry and activation of calcineurin resulting in CRTC dephosphorylation, induced CRTC1 nuclear entry in WT and TRPV1 mutant DRG cells and served as a positive control (FIG. 14C).

Activation of L-type voltage-gated calcium channels (LVGCCs) provokes robust calcium currents in neurons and results in hippocampal CRTC1 shuttling (Ch'ng et al., Cell 150:207-221, 2012). To test whether activation of TRPV1 and subsequent calcium entry were modulating CRTC1 directly, a natural TRPV1 agonist, capsaicin (Cap) was applied to DRG neurons and observed accumulation of CRTC1 in the nuclei of TRPV1-positive cells of WT DRGs cultures (FIG. 14C). The nuclear entry was similar to treatment with FSK and KCl (FIG. 14C). More importantly, Cap-induced CRTC1 shuttling was abolished in TRPV1 mutant DRG neurons (FIG. 14C). As an additional test of TRPV1's role in CRTC1 nuclear shuttling, blockade of TRPV1 by a selective chemical antagonist of TRPV1 (SB-366791) resulted in robust nuclear exclusion of CRTC1 in WT DRG neurons (FIG. 14D), much like that found in TRPV1 mutant DRG neurons.

These findings demonstrate that calcium entry through TRPV1 is sufficient to promote nuclear redistribution of CRTC1 and is likely to cause calcineurin activation. To examine the requirement of the calcineurin pathway downstream of TRPV1, we incubated the DRG neurons with the calcineurin inhibitor Cyclosporin A (CsA). As observed previously in hippocampal cells (Kovács et al., Proc. Natl. Acad. Sci. 104:4700-4705, 2007), CsA prevented FSK+KCl mediated CRTC1 nuclear accumulation in TRPV1 positive DRG neurons (FIG. 14D). CsA pretreatment also inhibited Cap ability to force CRTC1 into the nucleus in WT DRG neurons (FIG. 14D). Taken together, our data indicate that TRPV1 is a potent regulator of CRTC1 shuttling via calcineurin activity in both invertebrates and vertebrates.

Result: TRPV1 Regulates CREB Target Genes in DRGs

The ability of CRTC1 to shuttle to the nucleus under TRPV1 activity demonstrates the existence of a plastic transcriptional mechanism adapting rapidly to external outputs. The nuclear exclusion of CRTC1 in TRPV1 mutant DRG neurons suggests that CREB transcriptional activity is likely to be altered in the DRG neurons of TRPV1 mutant mice. Under inflammatory conditions, TRPV1 expressing DRG neurons utilize a CREB signaling cascade to induce neurogenic inflammation through the release of CGRP, by the binding of CREB onto the CGRP promoter (Nakanishi et al., Mol. Biol. Cell 21:2568-2577, 2010). It was hypothesized that in the TRPV1 mutant mice, CREB transcriptional activity is downregulated due to the nuclear exclusion of CRTC1. Indeed, mRNA expression analysis from DRG neurons revealed that previously characterized CREB target genes were reduced in long-lived TRPV1 mutants compared to wild-type animals (FIG. 14E).

Result: CGRP, but not Substance P, Regulates Insulin Secretion

The long-lived TRPV1 mutant mice enjoy a youthful metabolic program including improved glucose homeostasis due to increased insulin secretion upon glucose challenge. Because the DRG neurons form a dense meshwork innervating pancreatic β-cells (Akiba et al., Biochem. Biophys. Res. Commun. 321:219-225, 2004; Razavi et al., Autoimmune Diabetes. Cell 127:1123-1135, 2006), it was hypothesized that the TRPV1 neurons within the DRG might secrete a factor that inhibits insulin release from pancreatic β-cells, thereby establishing accurate insulin homeostasis. Furthermore, because CRTC1 localization is altered in the TRPV1 neurons, the secreted factor(s) that stem from TRPV1 neurons in the DRG might be under transcriptional control by CRTC1/CREB. In the analysis of CREB regulated genes within DRG neurons, the induction of calcitonin-related polypeptide α (ca1ca) and tachykinin 1 (tac1) transcripts, precursors of two TRPV1 secreted neuropeptides, CGRP and substance P, respectively, were reduced in the TRPV1 mutant mice (FIG. 14E). Therefore, we asked whether either substance P or CGRP could affect insulin secretion from pancreatic β-cells.

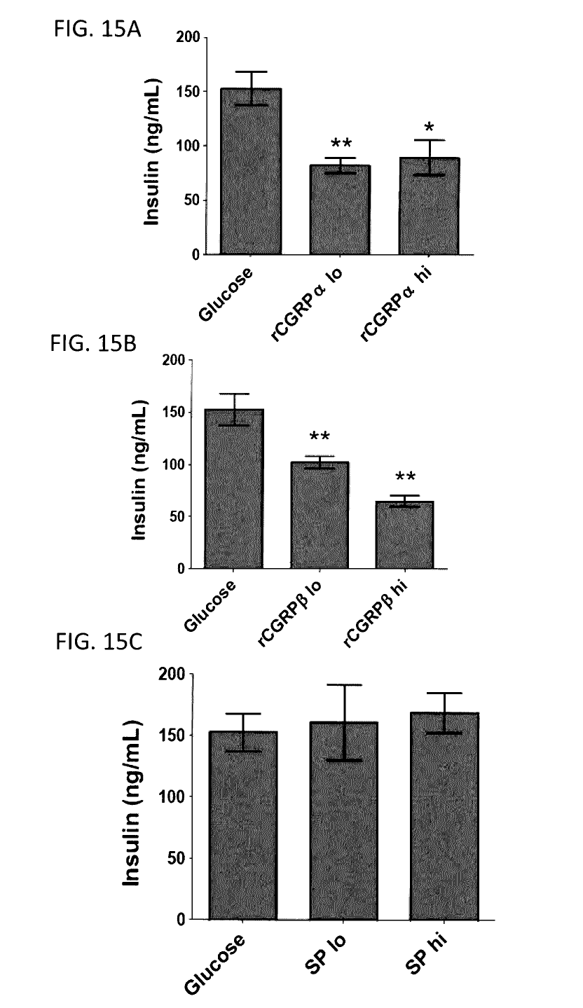

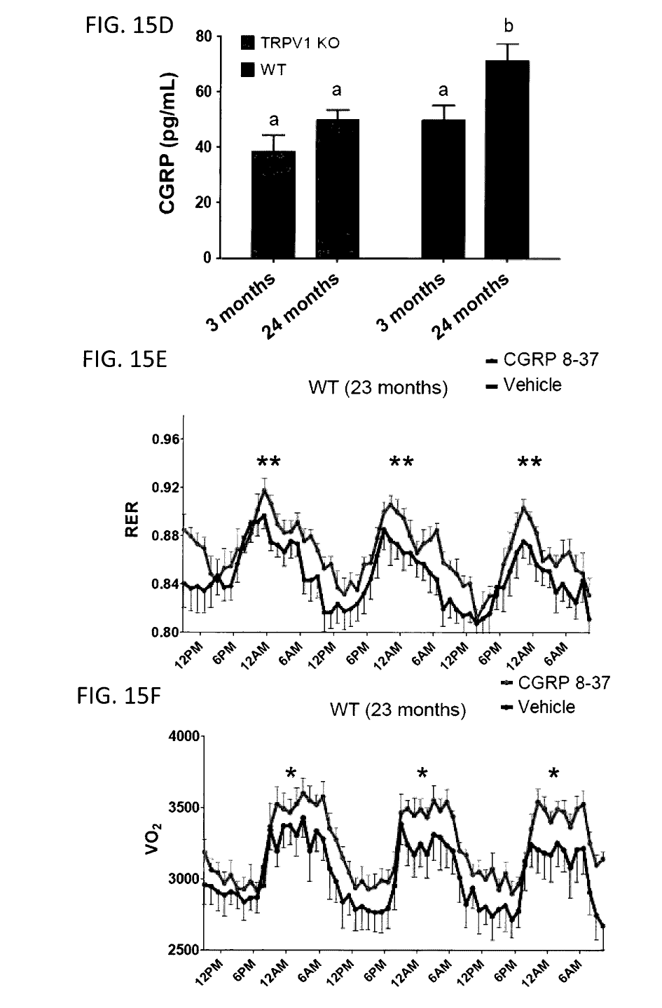

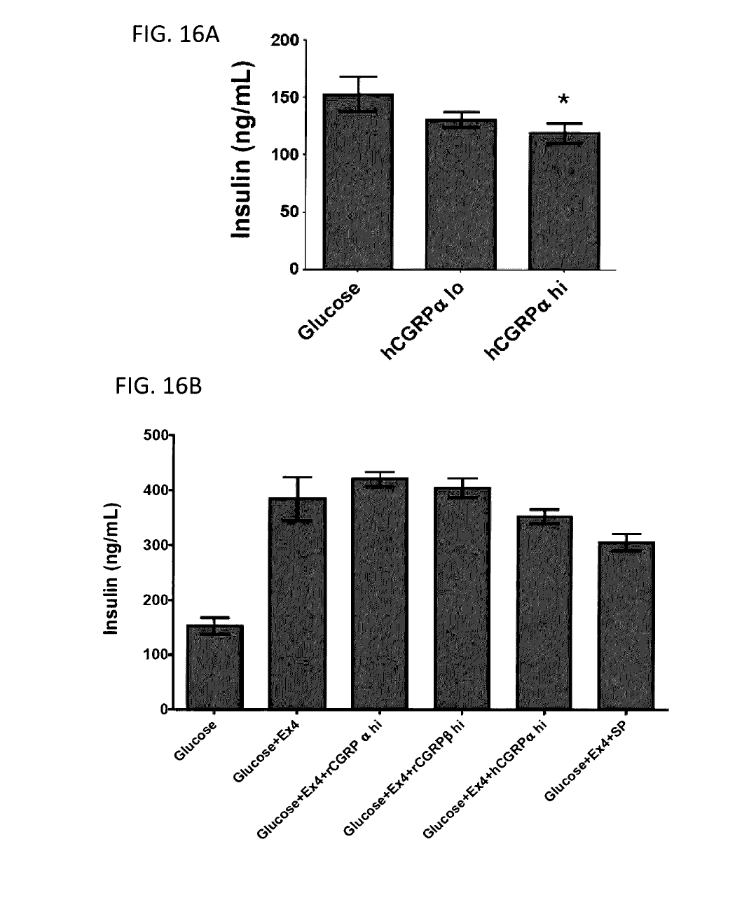

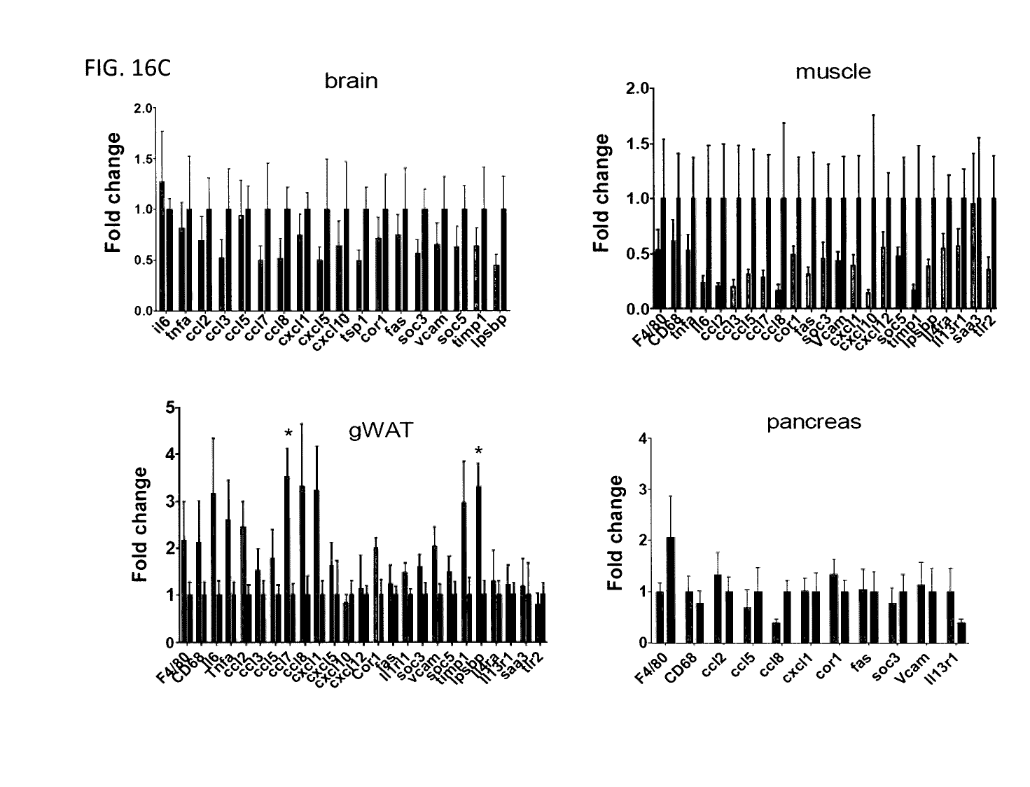

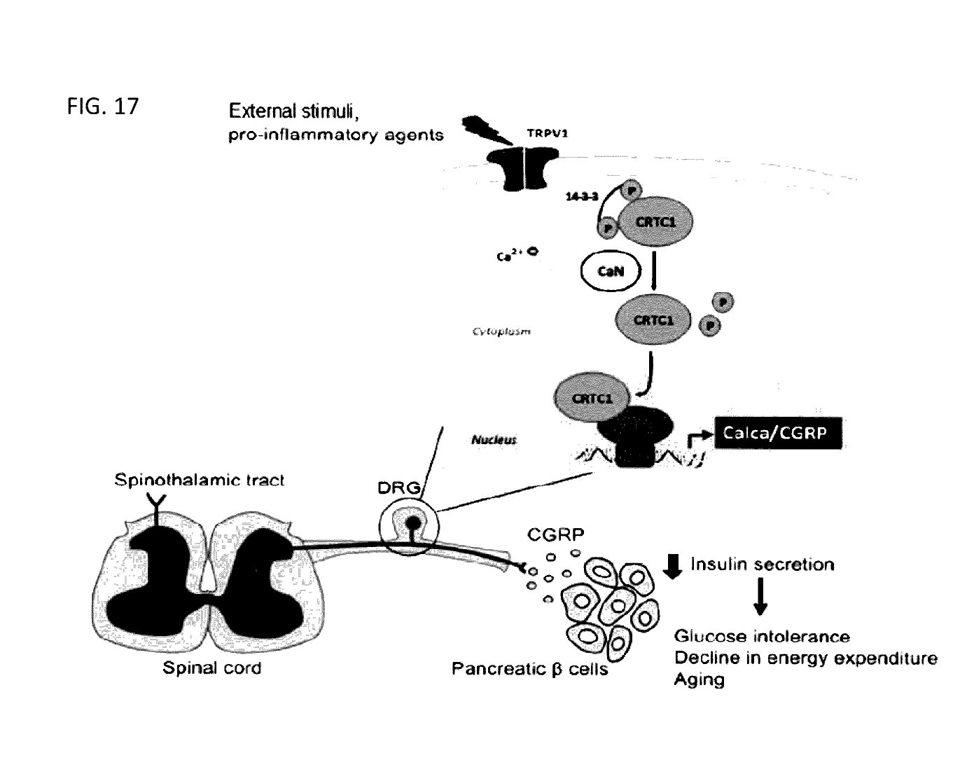

To mimic pancreatic β-cell glucose mediated insulin release, mouse insulinoma MIN6 cells were used (Huising et al., Proc. Natl. Acad. Sci. 107:912-917, 2010). When stimulated with 16.8 mM of glucose, MIN6 cells release up to 150 ng/ml of insulin. However, when the same cells are treated with doses ranging between 100 nM to 500 nM of either recombinant rat α-CGRP or β-CGRP (FIGS. 15 A, 15B), which are nearly identical at the amino acid level (Emeson et al., Nature 341:76-80, 1989), insulin release was greatly blunted. In particular 100 nM α-CGRP treatment significantly reduced insulin release by 46% and 500 nM β-CGRP by 58% of the maximum insulin response. Human α-CGRP also exhibited significant blockade of the insulin response, albeit to a lesser extent (FIG. 16A). Furthermore, treatment with 100 nM to 500 nM substance P did not alter insulin secretion of MIN6 cells, suggesting that CGRP's effect on insulin secretion is specifically mediated by a CGRP receptor (FIG. 15C). Interestingly, CGRP did not inhibit the potentiation of glucose-stimulated insulin secretion under high glucose by incretins as observed upon challenge with Exendin 4 (FIG. 16B). These data confirm previous observations that CGRP inhibits glucose-mediated insulin secretion from pancreatic β-cells (Ahren et al., Diabetologia 30:354-359, 1987; Kogire et al., Pancreas 6:459-463, 1991).

Result: CGRP Homeostasis Modulates Metabolic Health

The pathogenesis of type 2 diabetes involves a low-grade inflammation, which might continuously stimulate TRPV1 neurons and therefore putatively sustain CGRP release and exacerbation of the inflammatory response (Suri and Szallasi, 2008). Consistent with these results, a trend towards lower levels of inflammatory markers including chemokines and pro-inflammatory genes in brain and muscle tissue from TRPV1 mutant mice was observed (FIG. 16C). Similarly, aging results in chronic low-grade inflammation (Woods et al., Aging Dis. 3:130-140, 2011) and increased levels of circulating CGRP are detected in aged rats. Taken together, decreased levels of circulating CGRP might be beneficial for improved metabolic function and longevity, while high levels might be detrimental. To test this hypothesis, circulating CGRP levels in serum were measured in control and TRPV1 mutant mice (FIG. 15D). As observed in rats, serum levels of CGRP increase 42% with age in WT mice (6 vs. 24 months). However, this increase was blocked in TRPV1 mutant mice. In fact, CGRP levels remain unchanged between young and old TRPV1 mutant mice.

To ask whether increased CGRP might be detrimental to health of older animals, we challenged old wild-type mice with the CGRP receptor antagonist, CGRP8-37 (Poyner et al., Br. J. Pharmacol. 124:1659-1666, 1998), in an effort to restore CGRP homeostasis (FIGS. 15E and F). After 6 weeks of treatment, we found that the old mice displayed a reappearance of a more youthful metabolic circadian shift from night to day indicative of metabolic rejuvenation as measured by their RER, contrary to vehicle control mice. As observed in the TRPV1 mutant mice, oxygen consumption was also increased in CGRP8-37 treated mice compared to controls. These data indicate that pharmacological inhibition of the CGRP pathway is sufficient to restore metabolic youth in old animals and counteract the natural CGRP accumulation with age that is detrimental to wild-type animals.

### DISCUSSION

Although it has been shown that genetic manipulation of the CRTC1/CREB pathway modulates aging of C. elegans and that disruption of chemosensory perception extends lifespan in worms and flies, whether similar mechanisms regulate mammalian longevity were unknown. The results presented here provide evidence that genetic deletion of TRPV1, an ion channel critical for nociception, extends mouse and C. elegans lifespan by regulating the activity of CRTC1 in peripheral sensory neurons. Given the existing precedents linking TRPV1 to metabolism, the insulin metabolism of these mice was characterized in depth. Using a combination of in vivo and in vitro approaches, it was observed that TRPV1 mutant animals have improved glucose tolerance and increased energy expenditure throughout aging, despite a mild insulin resistance. Except for the latter, these phenotypes could underlie the exceptional longevity of these mice. It should be noted that insulin resistance has been observed in a variety of long-lived mice associated with improved glucose tolerance and therefore is not necessarily an indicator of poor health or lifespan shortening. Energy expenditure, on the opposite, has emerged as a putative longevity biomarker, as associated with lifespan extension. Improved energy expenditure throughout aging prevents systemic damage associated from fat storage and fat metabolism by ensuring a healthier transition between carbohydrate and fat metabolism. Consistent with improved energy expenditure, TRPV1 mutant mice present beneficial effects on glucose tolerance and resistance to obesity induced by high fat diet. Interestingly, a decrease in cancer incidence of neoplastic lymphoid origin has been previously associated with increased energy expenditure and increased lifespan as observed in the TRPV1 mutant mice, reinforcing the connection between improved energy expenditure and youthful aging.

Because CGRP is secreted locally from TRPV1 fibers in the pancreas, the data herein argue in favor of a role of TRPV1 on lifespan by modulating peripheral nervous system function, rather than for a role in the brain where TRPV1 is also present and affects hippocampal synaptic plasticity. Additionally, the weak presence of TRPV1 in neuroglia could underlie a contribution of glial cells to assist DRG neurons in regulating insulin secretion from pancreatic islets. Consistent with peripheral neuronal control, previous research supports a direct role of TRPV1 neurons in antagonizing insulin secretion in the β-cells through release of CGRP, suggesting that the improved cognitive function observed in TRPV1 mutant mice is more likely due to improved health span in these mice rather than the loss of TRPV1 in the hippocampus.

Disruption of calcium homeostasis in neurons is observed with aging and documented in the context of age-related hippocampal dysfunction, a highly age sensitive structure. TRPV1 activation induces calcium influx in neurons, leading to calcium-mediated signal transduction, including activation of CaMK and PKC isoenzymes. Therefore, preserving calcium balance by disrupting TRPV1 signaling is likely to be beneficial. Because calcium- and cAMP-dependent pathways that control gene expression share many common players and points of cross-talk ultimately leading in the phosphorylation of CREB at serine 133 and transcriptional activation, the CREB pathway regulated by its coactivator CRTC1, constitutes a highly likely candidate to integrate TRPV1 signals. Findings in both C. elegans and mouse primary DRG neurons strongly support a role for CRTC in shuttling in and out the nucleus in a calcineurin dependent manner to mediate calcium- and cAMP-dependent, phosphorylation-independent activation of the CREB transcription factor. The results further demonstrate that the silencing of CREB transcriptional regulation affects the synthesis of the CREB target CGRP.

CGRP levels increase drastically with age, and could contribute to the development of age-associated type 2 diabetes by blocking insulin secretion from β-cells. Consistent with this hypothesis, low grade inflammation is associated with age-onset T2D and pro-inflammatory agents might stimulate TRPV1 neurons causing sustained CGRP release and a higher state of inflammation leading to aggravated diabetes. Therefore sustained high CGRP levels are likely to negatively impact on β-cell function and metabolic health. In contrast, the maintenance of low CGRP levels achieved by the TRPV1 mutant mice or drug treatment with the CGRP receptor antagonist CGRP8-37, is associated with a youthful metabolism in old mice, a delay in age associated disease and increased longevity.

Taken together these findings illustrate that TRPV channels function in sensory neurons as an evolutionary conserved system integrating multiple sensory inputs and transducing them into neuroendocrine signals that promote longevity by adjusting the metabolic activity through the CRTC1/CREB circuit. In particular, these data highlight the role of the neuropeptide CGRP as a critical neuroendocrine regulator of longevity in mammals and possible biomarker of predictive lifespan and healthspan. Interestingly, the extremely long-lived naked mole rat, which lives over 30 years, is naturally lacking CGRP in DRGs (Park et al., J. Comp. Neurol. 465, 104-120, 2003). The pharmacological manipulation of TRPV1 and/or CGRP might not only be useful for pain but also to improve glucose homeostasis and aging. Consistent with this idea, diets rich in capsaicin, which can over stimulate TRPV1 neurons and cause their death, have long been linked to lower incidents of diabetes and metabolic dysregulation in humans (Westerterp-Plantenga et al., Int. J. Obes. 2005 29, 682-688, 2005).

TABLE 8

| Primer | Sequences | | | | |
|---|---|---|---|---|---|
| 18s | 4308329 (Applied | | F i11r11* | AGGCTCTC | (SEQ ID NO: |
| | Biosystems) | | | ACTTCTTG | 103) |
| | | | | GCTGA | |
| F β-actin | CTAAGGCCAAC | (SEQ ID NO: 49) | R i11r11* | GGGACACT | (SEQ ID NO: |
| | CGTGAAAAG | | | CCTTACTT | 104) |
| | | | | GGTTGT | |
| R β-actin | ACCAGAGGCAT | (SEQ ID NO: 50) | F i14ra* | TGGATCTG | (SEQ ID NO: |
| | ACAGGGACA | | | GGAGCATC | 105) |
| | | | | AAGGT | |
| F adrb3* | GGCCCTCTCTAG | (SEQ ID NO: 51) | R i14ra* | TGGAAGTG | (SEQ ID NO: |
| | TTCCCAG | | | CGGATGTA | 106) |
| | | | | GTCAG | |
| R adrb3* | TAGCCATCAAA | (SEQ ID NO: 52) | F i16* | TAGTCCTT | (SEQ ID NO: |
| | CCTGTTGAGC | | | CCTACCCC | 107) |
| | | | | AATTTCC | |
| F amy | AGTGGAATGGC | (SEQ ID NO: 53) | R i16* | TTGGTCCT | (SEQ ID NO: |
| | GAGAAGATG | | | TAGCCACT | 108) |
| | | | | CCTTC | |
| R amy | ACAAGGGCTCT | (SEQ ID NO: 54) | F i113r1* | TCAGCCAC | (SEQ ID NO: |
| | GTCAGAAGG | | | CTGTGACG | 109) |
| | | | | AATTT | |
| F ap2 | ACACCGAGATT | (SEQ ID NO: 55) | F i113r1* | TGAGAGTG | (SEQ ID NO: |
| | TCCTTCAAACTG | | | CAATTTGG | 110) |
| | | | | ACTGG | |
| R ap2 | CCATCTAGGGTT | (SEQ ID NO: 58) | F ins2 | GCTCTCTA | (SEQ ID NO: |
| | ATGATGCTCTTC | | | CCTGGTGT | 111) |
| | A | | | GTGGG | |
| F arc | GGTAAGTGCCG | (SEQ ID NO: 59) | R ins2 | CAAGGTCT | (SEQ ID NO: |
| | AGCTGAGATG | | | GAAGGTCA | 112) |
| | | | | CCTGC | |
| R arc | CGACCTGTGCA | (SEQ ID NO: 60) | F irs2 | GTCCAGGC | (SEQ ID NO: |
| | ACCCTTTC | | | ACTGGAGC | 113) |
| | | | | TTT | |
| F bdnf | TCATACTTCGGT | (SEQ ID NO: 61) | R irs2 | GCGCTTCA | (SEQ ID NO: |
| | TGCATGAAGG | | | CTCTTTCA | 114) |
| | | | | CGA | |
| R bdnf | AGACCTCTCGA | (SEQ ID NO: 62) | F lpsbp* | GATCACCG | (SEQ ID NO: |
| | ACCTGCCC | | | ACAAGGGC | 115) |
| | | | | CTG | |
| F calca | CAGTGCCTTTGA | (SEQ ID NO: 63) | R lpsbp* | GGCTATGA | (SEQ ID NO: |
| | GGTCAATCT | | | AACTCGTA | 116) |
| | | | | CTGCC | |
| R calca | CCAGCAGGCGA | (SEQ ID NO: 64) | F nos1 | CCCAACGT | (SEQ ID NO: |
| | ACTTCTTCTT | | | CATTTCTG | 117) |
| | | | | TCCGT | |
| F ccl2* | TTAAAAACCTG | (SEQ ID NO: 65) | R nos1 | TCTACCAG | (SEQ ID NO: |
| | GATCGGAACCA | | | GGGCCGAT | 118) |
| | A | | | CATT | |
| R ccl2* | GCATTAGCTTCA | (SEQ ID NO: 66) | F nr4a2 | TCAGAGCC | (SEQ ID NO: |
| | GATTTACGGGT | | | CACGTCGA | 119) |
| | | | | TT | |
| F ccl3* | TTCTCTGTACCA | (SEQ ID NO: 67) | R nr4a2 | TAGTCAGG | (SEQ ID NO: |
| | TGACACTCTGC | | | GTTTGCCT | 120) |
| | | | | GGAA | |
| R ccl3* | CGTGGAATCTTC | (SEQ ID NO: 68) | F nr4a3 | TGCGTGCA | (SEQ ID NO: |
| | CGGCTGTAG | | | AGCCCAGT | 121) |
| | | | | ATAG | |
| F ccl5* | GCTGCTTTGCCT | (SEQ ID NO: 69) | R nr4a3 | TGGTGTAT | (SEQ ID NO: |
| | ACCTCTCC | | | TCCGAGCC | 122) |
| | | | | ATAAGT | |
| R ccl5* | TCGAGTGACAA | (SEQ ID NO: 70) | F pgc1α | CCCTGCCA | (SEQ ID NO: |
| | ACACGACTGC | | | TTGTTAAG | 123) |
| | | | | ACC | |
| F ccl7* | GCTGCTTTCAGC | (SEQ ID NO: 71) | R pgc1α | TGCTGCTG | (SEQ ID NO: |
| | ATCCAAGTG | | | TTCCTGTT | 124) |
| | | | | TTC | |
| R ccl7* | CCAGGGACACC | (SEQ ID NO: 72) | F ppar-γ | CACAAGAG | (SEQ ID NO: |
| | GACTACTG | | | CTGACCCA | 125) |
| | | | ATGGT | | |
| F ccl8* | TCTACGCAGTG | (SEQ ID NO: 73) | R ppar-γ | GATCGCAC | (SEQ ID NO: |
| | CTTCTTTGCC | | | TTTGGTATT | 126) |
| | | | | CTTGGA | |
| R ccl8* | AAGGGGGATCT | (SEQ ID NO: 74) | F | CAGCACGG | (SEQ ID NO: |
| | TCAGCTTTAGTA | | prdm16 | TGAAGCCA | 127) |
| | | | | TTC | |
| F CD68* | TTCTCCAGCTGT | (SEQ ID NO: 75) | R | GCGTGCAT | (SEQ ID NO: |
| | TCACCTTGACCT | | prdm16 | CCGCTTGT | 128) |
| | | | | G | |
| R CD68* | GTTGCAAGAGA | (SEQ ID NO: 76) | F rgs2 | AGAAAATG | (SEQ ID NO: |
| | AACATGGCCCG | | | AAGCGGAC | 129) |
| | AA | | | ACTCTT | |
| F cfos | CAGCCTTTCCTA | (SEQ ID NO: 77) | R rgs2 | TTGCCAGT | (SEQ ID NO: |
| | CTACCATTCC | | | TTTGGGCT | 130) |
| | | | | TC | |
| R cfos | ACAGATCTGCG | (SEQ ID NO: 78) | F saa3* | TGCCATCA | (SEQ ID NO: |
| | CAAAAGTCC | | | TTCTTTGC | 131) |
| | | | | ATCTTGA | |
| F cidea | TGCTCTTCTGTA | (SEQ ID NO: 79) | R saa3* | CCGTGAAC | (SEQ ID NO: |
| | TCGCCCAGT | | | TTCTGAAC | 132) |
| | | | | AGCCT | |
| R cidea | GCCGTGTTAAG | (SEQ ID NO: 80) | F socs3* | ATGGTCAC | (SEQ ID NO: |
| | GAATCTGCTG | | | CCACAGCA | 133) |
| | | | | AGTTT | |
| F cor 1* | AGCCTGGCAAC | (SEQ ID NO: 81) | R socs3* | TCCAGTAG | (SEQ ID NO: |
| | TACTCTGACA | | | AATCCGCT | 134) |
| | | | | CTCCT | |
| R cor 1* | GAAGCACGTTC | (SEQ ID NO: 82) | F socs5* | GAGGGAG | (SEQ ID NO: |
| | TTGTTAGGCA | | | GAAGCCGT | 135) |
| | | | | AATGAG | |
| F cxcl1* | CTGGGATTCAC | (SEQ ID NO: 83) | R socs5* | CGGCACAG | (SEQ ID NO: |
| | CTCAAGAACAT | | | TTTTGGTT | 136) |
| | | | | C | CCG |
| R cxcl1* | CAGGGTCAAGG | (SEQ ID NO: 84) | F tad | AGCCTCAG | (SEQ ID NO: |
| | CAAGCCTC | | | CAGTTCTT | 137) |
| | | | | TGGA | |
| F cxcl5* | TGCGTTGTGTTT | (SEQ ID NO: 85) | R tad | TCTGGCCA | (SEQ ID NO: |
| | GCTTAACCG | | | TGTCCATA | 138) |
| | | | | AAGAG | |
| R cxcl5* | AGCTATGACTTC | (SEQ ID NO: 86) | F timp1* | GCAACTCG | (SEQ ID NO: |
| | CACCGTAGG | | | GACCTGGT | 139) |
| | | | | CATAA | |
| F cxcl10* | CCAAGTGCTGC | (SEQ ID NO: 87) | R timp1* | CGGCCCGT | (SEQ ID NO: |
| | CGTCATTTTC | | | GATGAGAA | 140) |
| | | | | ACT | |
| R cxcl10* | GGCTCGCAGGG | (SEQ ID NO: 88) | F tlr2* | GCAAACGC | (SEQ ID NO: |
| | ATGATTTCAA | | | TGTTCTGC | 141) |
| | | | | TCAG | |
| F cxcl12* | TGCATCAGTGA | (SEQ ID NO: 89) | R tlr2* | AGGCGTCT | (SEQ ID NO: |
| | CGGTAAACCA | | | CCCTCTAT | 142) |
| | | | | TGTATT | |
| R cxcl12* | TTCTTCAGCCGT | (SEQ ID NO: 90) | F tnfa* | CCCTCACA | (SEQ ID NO: |
| | GCAACAATC | | | CTCAGATC | 143) |
| | | | | ATCTTCT | |
| F dio2 | CAGTGTGGTTG | (SEQ ID NO: 91) | R tnfa* | GCTACGAC | (SEQ ID NO: |
| | CACGTCTCCAAT | | | GTGGGCTA | 144) |
| | C | | | CAG | |
| R dio2 | TGAACCAAAGT | (SEQ ID NO: 92) | F ucn3 | GCTGTGCC | (SEQ ID NO: |
| | TGACCACCAG | | | CCTCGACC | 145) |
| | | | | T | |
| F egr1 | TCGGCTCCTTTC | (SEQ ID NO: 93) | R ucn3 | TGGGCATC | (SEQ ID NO: |
| | CTCACTCA | | | AGCATCGC | 146) |
| | | | | T | |
| R egr1 | CTCATAGGGTT | (SEQ ID NO: 94) | F ucpl | ACTGCCAC | (SEQ ID NO: |
| | GTTCGCTCGG | | | ACCTCCAG | 147) |
| | | | | TCATT | |
| F fas* | AATCGCCTATG | (SEQ ID NO: 95) | R ucp1 | CTTTGCCT | (SEQ ID NO: |
| | GTTGTTGACC | | | CACTCAGG | 148) |
| | | | | ATTGG | |
| R fas* | TTGGTATGGTTT | (SEQ ID NO: 96) | F ucp2 | CAGGTCAC | (SEQ ID NO: |
| | CACGACTGG | | | TGTGCCCT | 149) |
| | | | | TACCA | |
| F F4/80* | CCCCAGTGTCCT | (SEQ ID NO: 97) | R ucp2 | CACTACGT | (SEQ ID NO: |
| | TACAGAGTG | | | TCCAGGAT | 150) |
| | | | | CCCAA | |
| R F4/80* | GTGCCCAGAGT | (SEQ ID NO: 98) | F uspl | GTGCCTGA | (SEQ ID NO: |
| | GGATGTCT | | | CAGTGCAG | 151) |
| | | | | AATC | |
| gapdh | 4308313 (Applied | | R usp1 | CACTGCCC | (SEQ ID NO: |
| | Biosystems) | | | AGGTACAG | 152) |
| | | | | GAAG | |
| F gcg | TCACAGGGCAC | (SEQ ID NO: 99) | F vcam* | AGTTGGGG | (SEQ ID NO: |
| | ATTCACCAG | | | ATTCGGTT | 153) |
| | | | | GTTCT | |
| R gcg | CATCATGACGTT | (SEQ ID NO: | R vcam* | CCCCTCAT | (SEQ ID NO: |
| | TGGCAATGTT | 100) | | TCCTTACC | 154) |
| | | | | ACCC | |
| F hprt | TCCTCCTCAGAC | (SEQ ID NO: | | | |
| | CGCTTTT | 101) | | | |
| R hprt | CCTGGTTCATCA | (SEQ ID NO: | | | |
| | TCGCTAATC | 102) | | | |

TABLE 9
Comparative survival characteristics of TRPV1 mutant and WT mice.

| | | | | | Log-Rank | Max |
|---|---|---|---|---|---|---|
| | n | Median | Min-Max | Mean | Test | Lifespan |
| Females | | | | | | |
| TRPV1 KO | 48 | 946 | 685-1156 | 957.48 ± 16.66 | _{χ}2 = 18.35, | p < 0.001 |
| | | | | | p < 0.0001 | |
| WT | 42 | 816 | 375-1049 | 827.98 ± 18.47 | | |
| Males | | | | | | |
| TRPV1 KO | 38 | 1038 | 780-1226 | 1036.31 ± 23.32 | _{χ}2 = 9.92, | ns |
| | | | | | p < 0.001 | |
| WT | 32 | 927.5 | 614-1187 | 937 ± 24.79 | | |

TABLE 10
Necropsy analysis of non-neoplastic diseases in TRPV1
KO and wild-type mice. Results reported as percentage
(total number) of mice with the disease.

| | | TRPV1 KO | Wild Type |
|---|---|---|---|
| Non-neoplastic diseases | Location | % (n) | % (n) |
| Acidophilic macrophage | Lungs | 0.0 (0) | 4.7 (2) |
| pneumonia | | | |
| Bronchopneumonia | Lungs | 3.0 (2) | 0.0 (0) |
| Heart lesions | Heart | 9.1 (6) | 7.0 (3) |
| Atrial thrombosis | Heart | 4.5 (3) | 0.0 (0) |
| Amyloidosis | Kidney | 7.6 (5) | 9.3 (4) |
| Nephropathy | Kidney | 10.6 (7) | 9.3 (4) |
| Glomerulonephritis | Kidney | 1.5 (1) | 2.3 (1) |
| Obstructive uropathy | Bladder | 7.6 (5) | 0 (0) |
| Hepatic inflammation | Liver | 4.5 (2) | 4.7 (2) |
| Biliary hyperplasia | Liver | 1.5 (1) | 2.3 (1) |
| Rectal prolapse | Rectum | 6.1 (4) | 2.3 (1) |
| Seminal vesiculitis, cystic | Seminal | 9.1 (6) | 23.3 (10) |
| degeneration | vesicles | | |

TABLE 11
Genome scan analysis of lifespan mice compared to C57BL6 genetic
background. SNPs polymorphism between B6 and 129 strains were
evaluated for TRPV1 mutant and WT control mice.

| | Genotype | | | | | | | | |
|---|---|---|---|---|---|---|---|---|---|
| | %100 | %100 | %100 | %100 | | | | | |
| | TRPV1 | TRPV1 | TRPV1 | TRPV1 | %100 | %100 | %100 | | |
| | KO | KO | KO | KO | WT | WT | WT | %100 | %0 |
| Chromosome -bp | 1 | 2 | 3 | 4 | 5 | 6 | 7 | B6 | 129 |
| 01-005230167-M | C | C | C | C | C | C | C | C | T |
| 01-026072256-M | G | G | G | G | G | G | G | G | T |
| 01-046003967-M | A | A | A | A | A | A | A | A | C |
| 01-065042402-M | G | G | G | G | G | G | G | G | A |
| 01-086182722-M | A | A | A | A | A | A | A | A | G |
| 01-102073421-M | G | G | G | G | G | G | G | G | C |
| 01-122978406-M | G | G | G | G | G | G | G | G | T |
| 01-143006008-M | A | A | A | A | A | A | A | A | G |
| 01-162977516-M | G | G | G | G | G | G | G | G | A |
| 01-178701443-M | A | A | A | A | A | A | A | A | G |
| 01-193173300-M | G | G | G | G | G | G | G | G | A |
| 02-003179310-M | G | G | G | G | G | G | G | G | A |
| 02-020551513-M | A | A | A | A | A | A | A | A | G |
| 02-041042651-M | C | C | C | C | C | C | C | C | G |
| 02-061161692-M | T | T | T | T | T | T | T | T | C |
| 02-078062303-M | T | T | T | T | T | T | T | T | G |
| 02-101074874-M | A | A | A | A | A | A | A | A | G |
| 02-119931810-M | A | A | A | A | A | A | A | A | T |
| 02-139603599-M | T | T | T | T | T | T | T | T | A |
| 02-161858323-M | C | C | C | C | C | C | C | C | A |
| 02-179086475-M | G | G | G | G | G | G | G | G | T |
| 03-011350737-M | G | G | G | G | G | G | G | G | C |
| 03-032431792-M | G | G | G | G | G | G | G | G | A |
| 03-053161686-M | T | T | T | T | T | T | T | T | C |
| 03-073214161-M | A | A | A | A | A | A | A | A | T |
| 03-088155864-N | T | T | T | T | T | T | T | T | C |
| 03-110268748-M | C | C | C | C | C | C | C | C | T |
| 03-131286062-N | C | C | C | C | C | C | C | C | T |
| 03-151125653-M | A | A | A | A | A | A | A | A | T |
| 03-160287200-M | G | G | G | G | G | G | G | G | A |
| 04-003163167-M | G | G | G | G | G | G | G | G | T |
| 04-022875524-M | C | C | C | C | C | C | C | C | T |
| 04-039199957-M | C | C | C | C | C | C | C | C | G |
| 04-061409675-M | G | G | G | G | G | G | G | G | T |
| 04-084241978-M | T | T | T | T | T | T | T | T | C |
| 04-106314110-M | C | C | C | C | C | C | C | C | A |
| 04-120122875-M | G | G | G | G | G | G | G | G | A |
| 04-141084977-M | A | A | A | A | A | A | A | A | G |
| 04-151168886-M | G | G | G | G | G | G | G | G | T |
| 05-003188964-M | C | C | C | C | C | C | C | C | T |
| 05-023890459-M | C | C | C | C | C | C | C | C | G |
| 05-044010971-M | T | T | T | T | T | T | T | T | G |
| 05-065734378-N | A | A | A | A | A | A | A | A | G |
| 05-082050497-M | C | C | C | C | C | C | C | C | A |
| 05-106058057-M | T | T | T | T | T | T | T | T | C |
| 05-125054661-M | T | T | T | T | T | T | T | T | C |
| 05-139966164-M | T | T | T | T | T | T | T | T | C |
| 05-149044358-M | C | C | C | C | C | C | C | C | A |
| 06-003167392-M | C | C | C | C | C | C | C | C | T |
| 06-017592278-N | G | G | G | G | G | G | G | G | T |
| 06-045126062-M | A | A | A | A | A | A | A | A | G |
| 06-062599520-M | A | A | A | A | A | A | A | A | G |
| 06-083216741-M | G | G | G | G | G | G | G | G | T |
| 06-106057600-M | C | C | C | C | C | C | C | C | T |
| 06-122941044-M | T | T | T | T | T | T | T | T | A |
| 06-135955068-M | C | C | C | C | C | C | C | C | A |
| 06-149052281-M | C | C | C | C | C | C | C | C | G |
| 07-004201219-N | A | A | A | A | A | A | A | A | G |
| 07-022997618-M | C | C | C | C | C | C | C | C | T |
| 07-044964980-M | G | G | G | G | G | G | G | G | C |
| 07-064095712-M | G | G | G | G | G | G | G | G | T |
| 07-085095955-M | C | C | C | C | C | C | C | C | T |
| 07-106153798-M | G | G | G | G | G | G | G | G | T |
| 07-135024189-M | C | C | C | C | C | C | C | C | T |
| 08-003089774-M | T | T | T | T | T | T | T | T | C |
| 08-025034424-M | C | C | C | C | C | C | C | C | T |
| 08-045080531-M | T | T | T | T | T | T | T | T | C |
| 08-067470102-N | G | G | G | G | G | G | G | G | C |
| 08-086985036-M | C | C | C | C | C | C | C | C | T |
| 08-107088585-M | A | A | A | A | A | A | A | A | G |
| 08-123021323-M | T | T | T | T | T | T | T | T | C |
| 09-003938578-M | T | T | T | T | T | T | T | T | C |
| 09-016198772-M | G | G | G | G | G | G | G | G | T |
| 09-034961903-M | G | G | G | G | G | G | G | G | A |
| 09-052894456-M | C | C | C | C | C | C | C | C | A |
| 09-077792491-M | T | T | T | T | T | T | T | T | A |
| 09-098865563-M | T | T | T | T | T | T | T | T | C |
| 09-115037423-M | G | G | G | G | G | G | G | G | A |
| 09-125082704-M | G | G | G | G | G | G | G | G | A |
| 10-003233934-M | G | G | G | G | G | G | G | G | C |
| 10-023127952-M | A | A | A | A | A | A | A | A | G |
| 10-034328321-G | C | C | C | C | C | C | C | C | A |
| 10-053898997-M | G | G | G | G | G | G | G | G | C |
| 10-072471838-G | A | A | A | A | A | A | A | A | G |
| 10-093476458-N | T | T | T | T | T | T | T | T | C |
| 10-117629082-M | A | A | A | A | A | A | A | A | G |
| 10-129117100-M | G | G | G | G | G | G | G | G | A |
| 11-005661856-N | T | T | T | T | T | T | T | T | A |
| 11-024515722-M | G | G | G | G | G | G | G | G | A |
| 11-047224208-M | C | C | C | C | C | C | C | C | A |
| 11-090199967-M | T | T | T | T | T | T | T | T | C |
| 11-111226923-M | A | A | A | A | A | A | A | A | C |
| 11-122195777-M | C | C | C | C | C | C | C | C | T |
| 12-003567042-M | G | G | G | G | G | G | G | G | T |
| 12-022232159-M | C | C | C | C | C | C | C | C | G |
| 12-041270227-M | A | A | A | A | A | A | A | A | G |
| 12-061271925-M | A | A | A | A | A | A | A | A | T |
| 12-069389027-M | A | A | A | A | A | A | A | A | G |
| 12-092440423-M | G | G | G | G | G | G | G | G | C |
| 12-107536049-N | A | A | A | A | A | A | A | A | G |
| 12-112276179-M | A | A | A | A | A | A | A | A | T |
| 13-003966099-M | A | A | A | A | A | A | A | A | G |
| 13-023105764-M | A | A | A | A | A | A | A | A | G |
| 13-043995023-N | T | T | T | T | T | T | T | T | G |
| 13-065339791-N | C | C | C | C | C | C | C | C | T |
| 13-084819885-M | G | G | G | G | G | G | G | G | A |
| 13-101929792-M | C | C | C | C | C | C | C | C | T |
| 13-117094028-M | G | G | G | G | G | G | G | G | A |
| 14-005055006-M | T | T | T | T | T | T | T | T | A |
| 14-024063411-M | A | A | A | A | A | A | A | A | G |
| 14-040017031-M | G | G | G | G | G | G | G | G | C |
| 14-060626617-N | T | T | T | T | T | T | T | T | C |
| 14-077156430-M | C | C | C | C | C | C | C | C | T |
| 14-097042967-M | A | A | A | A | A | A | A | A | G |
| 14-114403652-M | C | C | C | C | C | C | C | C | T |
| 15-003094890-M | A | A | A | A | A | A | A | A | G |
| 15-020280219-M | G | G | G | G | G | G | G | G | T |
| 15-043015319-M | G | G | G | G | G | G | G | G | C |
| 15-059754938-N | C | C | C | C | C | C | C | C | T |
| 15-102788257-N | G | G | G | G | G | G | G | G | T |
| 16-005053446-C | G | G | G | G | G | G | G | G | T |
| 16-019621494-C | A | A | A | A | A | A | A | A | G |
| 16-037994236-C | G | G | G | G | G | G | G | G | A |
| 16-057482753-C | A | A | A | A | A | A | A | A | G |
| 16-075684315-C | T | T | T | T | T | T | T | T | C |
| 16-096070057-C | C | C | C | C | C | C | C | C | T |
| 17-003335010-M | G | G | G | G | G | G | G | G | C |
| 17-023157746-M | A | A | A | A | A | A | A | A | T |
| 17-043164647-M | G | G | G | G | G | G | G | G | A |
| 17-066532305-N | T | T | T | T | T | T | T | T | G |
| 17-083294998-M | T | T | T | T | T | T | T | T | A |
| 17-092673068-N | T | T | T | T | T | T | T | T | G |
| 18-005066417-M | T | T | T | T | T | T | T | T | C |
| 18-022053385-M | A | A | A | A | A | A | A | A | G |
| 18-043081750-M | C | C | C | C | C | C | C | C | T |
| 18-054835717-M | T | T | T | T | T | T | T | T | A |
| 18-067972780-M | T | T | T | T | T | T | T | T | C |
| 18-078813035-M | A | A | A | A | A | A | A | A | C |
| 18-090060367-M | G | G | G | G | G | G | G | G | T |
| 19-018239318-M | T | T | T | T | T | T | T | T | G |
| 19-021081804-M | A | A | A | A | A | A | A | A | G |
| 19-038092479-M | T | T | T | T | T | T | T | T | C |
| 19-048069669-N | C | C | C | C | C | C | C | C | T |
| 19-060823449-N | A | A | A | A | A | A | A | A | G |
| X-016114535-G | C | C | C | C | C | C | C | C | A |
| X-025132696-G | C | C | C | C | C | C | C | C | G |
| X-054650362-N | T | T | T | T | T | T | T | T | A |
| X-068494838-M | C | C | C | C | C | C | C | C | T |
| X-087817653-G | A | A | A | A | A | A | A | A | T |
| X-100413449-M | T | T | T | T | T | T | T | T | G |
| X-114340727-M | A | A | A | A | A | A | A | A | G |
| X-133535206-G | T | T | T | T | T | T | T | T | C |
| X-143466659-M | A | A | A | A | A | A | A | A | C |

TABLE 12
C. elegans lifespan analysis

| | | | | Repe- | |
|---|---|---|---|---|---|
| Strain | RNAi | Median | SEM | titions | p |
| N2 | GFP RNAi | 22.3 | 0.88 | 3 | |
| osm-9(ky10); | GFP RNAi | 28.7 | 1.45 | 4 | **0.001 |
| ocr-2(ak47) | | | | | |
| osm-9(ky10) | GFP RNAi | 23 | 1.22 | 4 | 0.09 |
| ocr-2(ak47) | GFP RNAi | 23 | 1 | 3 | *0.01 |
| CRTC1::rfp | GFP RNAi | 20.2 | 1.97 | 4 | |
| osm-9; ocr-2; | GFP RNAi | 26.8 | 2.17 | 4 | **0.001 |
| CRTC1::rfp | | | | | |
| CRTC1 | GFP RNAi | 18 | 3 | 2 | |
| (S76A, S79A) | | | | | |
| osm9; ocr2; | GFP RNAi | 16.5 | 1.5 | 2 | 0.05 |
| CRTC1 | | | | | |
| (S76A, S79A) | | | | | |
| CRTC1::rfp | tax-6 | 26 | 0 | 2 | |
| | RNAi | | | | |
| osm-9; ocr-2; | tax-6 | 23.5 | 0.5 | 2 | ***0.0001 |
| CRTC1::rfp | RNAi | | | | |

### Example 6

### Effects of Anti-CGRP Antagonist Antibody on the Metabolic Health in Diabetic Animals

The use of one or more anti-CGRP antagonist antibodies as described herein on the metabolic health such as responsiveness of glucose and insulin, respiratory exchange ratio and oxygen consumption in a diabetic animal is investigated. Compositions comprising one or more anti-CGRP antagonist antibodies can be used to increase glucose responsiveness, insulin secretion and metabolic health in a subject that is suffering from diabetes. The effects are investigated herein.

A diabetes rodent model, e.g., BKS-DB mice, is acquired for the study. Alternatively, diabetes of rodents can be induced by administering Streptozotocin (STZ) to the rodents following a standard protocol. The animals are housed in a group of 2-5 in a temperature (about 24±1° C.)—and humidity-controlled environment (about 50-60%) with about regular light and dark cycle following a clean house-keeping practice. The animals are provided free access to food and water.

All groups are administered either drug vehicle solution (PBS, 0.01% tween) or anti-CGRP antibody solution by injection. The groups include DM model control group administered with drug vehicle solution, anti-CGRP antibody group administered with different dosages of antibody. In addition, a group of wild type animals without the diabetes symptom is administered control vehicle solution. Specifically, rodents are injected intravenously (10 mg/kg, 3 mg/kg or 1 mg/kg of 4901 or G1 antibodies).

All animals are monitored closely for the amount of water and food consumed, amount of urine, general mental state, activity and hair color. All animals are weighed once weekly, and their survival over time are recorded. At specific time points such as 1, 2, 3, 4 weeks, animals are evaluated for glucose tolerance and responsiveness, as well as the insulin resistance.

After fasting for 5 hours, blood samples of the fasting animals in all groups are obtained from tail vein. A glucose tolerance test (GTT) is also conducted to assess the glucose tolerance and responsiveness of the animals. Animals from all groups are intraperitoneally administered glucose solution (2 g/kg weight). Blood samples in all groups are obtained from tail vein at time 0 (prior to glucose administration), 15 minutes, 0.5 hour, 1 hour, 1.5 hours, 2 hours and 2.5 hours after the administration of glucose solution. The serum/plasma level of glucose is measured. For the insulin tolerance test (ITT), 5 hour fasted rodents are injected with 1 u/kg of human insulin and glucose is measured as in the GTT. Insulin-stimulated signaling in liver and muscle tissues is performed as previously described in Jorden et al., 2011. The fasting serum/plasma level of glucose and insulin level, and their levels at each time point in both GTT and ITT tests are measured. Respiratory exchange ratio (RER) analysis and oxygen consumption are evaluated. The life span of the animals are also recorded and compared.

It is expected that anti-CGRP antagonist antibody will improve metabolic health and increase longevity in diabetic animals.

### Example 7

### Comparisons of the Anti-CGRP Antagonist Antibody and TRPV-1 Inhibition on the Metabolic Health in Diabetic Animals

The difference on the effects of anti-CGRP antagonist antibodies as described herein and TRPV-1 inhibition on metabolic health such as insulin secretion and glucose levels and tolerance in a diabetic animal is investigated. The effects are investigated herein.

A diabetes rodent model, e.g., BKS-DB mice, can be used. Alternatively, diabetes of rodents can be induced by administering Streptozotocin (STZ) to the rodents following a standard protocol. The animals are housed in a group of 2-5 in a temperature (about 24±1° C.)- and humidity-controlled environment (about 50-60%) with about regular light and dark cycle following a clean house-keeping practice. The animals are provided free access to food and water.

Groups of diabetic mice are administered a drug vehicle solution (PBS, 0.01% tween) containing either anti-CGRP antibody, or capsazepine (a TRPV-1 antagonist) by injection. For comparison, groups of wild type animals without the diabetes symptom receive similar treatments. All animals are monitored closely for the amount of water and food consumed, amount of urine, general mental state, activity and hair color. All animals are weighed once weekly. At specific time points such as 1, 2, 3, 4 weeks, animals are evaluated for glucose tolerance and responsiveness, as well as the insulin resistance.

After fasting for 5 hours, blood samples of the fasting animals in all groups are obtained from tail vein. A glucose tolerance test (GTT) is also conducted to assess the glucose tolerance and responsiveness of the animals. Animals from all groups are intraperitoneally administered glucose solution (2 g/kg weight). Blood samples in all groups are obtained from tail vein at time 0 (prior to glucose administration), 15 minutes, 0.5 hour, 1 hour, 1.5 hours, 2 hours and 2.5 hours after the administration of glucose solution. The serum/plasma level of glucose is measured. For the insulin tolerance test (ITT), 5 hour fasted rodents are injected with 1 u/kg of human insulin and glucose is measured as in the GTT. Insulin-stimulated signaling in liver and muscle tissues is performed as previously described in Jorden et al., 2011. The fasting serum/plasma level of glucose and insulin level, and their levels at each time point in both GTT and ITT tests are measured. Respiratory exchange ratio (RER) analysis and oxygen consumption are evaluated. The life span of the animals are also recorded and compared.

### Example 8

### Comparison of Different Anti-CGRP Antagonist Antibodies on the Metabolic Health and Longevity in Diabetic Animals

The difference on the effects of anti-CGRP antagonist antibodies obtained from different sources and vendors on insulin secretion and glucose levels and tolerance in a diabetic animal is investigated. The effects are investigated herein.

A diabetes rodent model, e.g., BKS-DB mice, can be used. Alternatively, diabetes of rodents can be induced by administering Streptozotocin (STZ) to the rodents following a standard protocol. The animals are housed in a group of 2-5 in a temperature (about 24±1° C.)- and humidity-controlled environment (about 50-60%) with about regular light and dark cycle following a clean house-keeping practice. The animals are provided free access to food and water.

Groups are administered drug vehicle solution (PBS, 0.01% tween), containing anti-CGRP antibody G1 (as described herein), ALD403 (an anti-CGRP antibody from Alder BioPharmaceuticals), or LY2951742 (an anti-CGRP antibody from Arteaus Therapeutics) by injection. The antibodies may be administered at different concentrations. For comparison, groups of wild type animals without the diabetes symptom receive similar treatments. All animals are monitored closely for the amount of water and food consumed, amount of urine, general mental state, activity and hair color. All animals are weighed once weekly. At specific time points such as 1, 2, 3, 4 weeks, animals are evaluated for glucose tolerance and responsiveness, as well as the insulin resistance. Respiratory exchange ratio (RER) analysis and oxygen consumption are evaluated. The life span of the animals are also recorded and compared.

After fasting for 5 hours, blood samples of the fasting animals in all groups are obtained from tail vein. A glucose tolerance test (GTT) is also conducted to assess the glucose tolerance and responsiveness of the animals. Animals from all groups are intraperitoneally administered glucose solution (2 g/kg weight). Blood samples in all groups are obtained from tail vein at time 0 (prior to glucose administration), 15 minutes, 0.5 hour, 1 hour, 1.5 hours, 2 hours and 2.5 hours after the administration of glucose solution. The serum/plasma level of glucose is measured. For the insulin tolerance test (ITT), 5 hour fasted rodents are injected with 1 u/kg of human insulin and glucose is measured as in the GTT. Insulin-stimulated signaling in liver and muscle tissues is performed as previously described in Jorden et al., 2011. The fasting serum/plasma level of glucose and insulin level, and their levels at each time point in both GTT and ITT tests are measured.

It is expected that all anti-CGRP antagonist antibodies will promote metabolic health such as increase glucose and insulin tolerance and responsiveness in diabetic animals, and will increase life span of the animals.

### Example 9

### Effects of Anti-CGRP Antagonist Antibody on a Diabetic Human

The use of anti-CGRP antagonist antibody as described herein on the metabolic health in a diabetic patient is investigated. A group of diabetes patients are enrolled for the study. All patients have similar diet and exercise habits, ages and/or weights. The weight of each patient is monitored daily. All patients are administered either a placebo or anti-CGRP antibody solution by injection.

Each patient is administered with a dose of at least about 225 mg of anti-CGRP antagonist antibody. The anti-CGRP antagonist antibody is supplied as a liquid formulation at a concentration of at least about 150 mg/mL. The dose is administered as a subcutaneous injection to the back of an upper arm of the subject's body. Alternatively, the dose may be provided to the subject via intravenous infusion. In such cases, 5.85 mL of 150 mg/mL anti-CGRP antibody may be combined with 0.9% Sodium Chloride Solution (Normal Saline) in an IV bag for a total volume of 130 mL in the bag. About 100 mL of the IV bag volume can be intravenously infused to the subject over the course of one hour, for a total dose of 225 mg. Dosing is repeated. All patients are asked to record the amount of water and food consumed, amount of urine, general mental state, and activity daily. After fasting for 8-12 hours, blood samples of the fasting patients in all groups are obtained at baseline (prior to antibody or placebo administration), and at specific time points after the administration of saline or drug. The fasting serum/plasma level of glucose is measured. A glucose tolerance test (GTT) and an insulin resistance test are also conducted to assess the improvement of the disease.

While embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

1. A method for treating a metabolic disorder, comprising administering to a subject suffering a metabolic disorder a therapeutically effective amount of one or more anti-calcitonin gene-related peptide (CGRP) antagonist antibodies.

2. The method of claim 1, wherein the metabolic disorder is weight gain, diabetes, and/or a cardiovascular disease.

3. The method of claim 1, wherein the metabolic disorder is diabetes.

4. The method of claim 1, wherein the metabolic disorder is a disorder characterized by inhibition of insulin secretion.

5. The method of claim 1, wherein the subject shows increased insulin secretion, increased glucose tolerance and increased longevity after said administering.

6. The method of claim 1, wherein the antibodies are human or humanized antibodies.

7. The method of claim 1, wherein the antibodies are monoclonal antibodies.

8. The method of claim 1, wherein the subject is human.

9. The method of claim 1, wherein at least one of the one or more anti-CGRP antagonist antibodies binds calcitonin gene-related peptide (CGRP) with a KD of 50 nM or less as measured by surface plasmon resonance at 37° C.

10. The method of claim 1, wherein at least one of the one or more anti-CGRP antagonist antibodies has a half life in vivo of at least 7 days.

11. The method of claim 1, wherein at least one of the one or more anti-CGRP antagonist antibodies specifically binds to the C-terminal region of CGRP.

12. The method of claim 11, wherein at least one of the one or more anti-CGRP antagonist antibodies specifically recognize the epitope defined by the sequence GSKAF (SEQ ID NO: 48).

13. The method of claim 1, wherein at least one of the one or more anti-CGRP antagonist antibodies comprises:
a VH domain having the amino acid sequence shown in SEQ ID NO: 1 or 155;
and/or
a VL domain having the amino acid sequence shown in SEQ ID NO: 2 or 156.

14. The method of claim 1, wherein at least one of the one or more anti-CGRP antagonist antibodies comprises: (a) CDR H1 as set forth in SEQ ID NO: 3, 33, or 170; CDR H2 as set forth in SEQ ID NO: 4 or 171; CDR H3 as set forth in SEQ ID NO: 5; CDR L1 as set forth in SEQ ID NO: 6; CDR L2 as set forth in SEQ ID NO: 7; and CDR L3 as set forth in SEQ ID NO: 8; (b) CDR H1 as set forth in SEQ ID NO: 157, 172, or 173; CDR H2 as set forth in SEQ ID NO: 22 or 174; CDR H3 as set forth in SEQ ID NO: 159; CDR L1 as set forth in SEQ ID NO: 160; CDR L2 as set forth in SEQ ID NO: 161; and CDR L3 as set forth in SEQ ID NO: 162; or (c) a variant of an antibody according to (a) as shown in Table 6.

15. The method of claim 1, wherein at least one of the one or more anti-CGRP antagonist antibodies comprises a VH domain having the amino acid sequence shown in SEQ ID NO: 1 and a VL domain having the amino acid sequence shown in SEQ ID NO: 2.

16. The method of claim 1, wherein at least one of the one or more anti-CGRP antagonist antibodies is produced by the expression vectors with ATCC Accession Nos. PTA-6867 and/or PTA-6866.

17. The method of claim 1, wherein at least one of the one or more anti-CGRP antagonist antibodies comprises:
the antibody G1 heavy chain full antibody amino acid sequence shown in SEQ ID NO: 11, with or without the C-terminal lysine; and the antibody G1 light chain full antibody amino acid sequence shown in SEQ ID NO: 12 and/or
the antibody G2 heavy chain full antibody amino acid sequence shown in SEQ ID NO: 165; and the antibody G2 light chain full antibody amino acid sequence shown in SEQ ID NO: 166.

18. The method of claim 1, wherein the one or more anti-CGRP antagonist antibodies are:
peripherally administered, or
administered orally, sublingually, via inhalation, transdermally, subcutaneously, intravenously, intra-arterially, intra-articularly, peri-articularly, locally and/or intramuscularly.

19. The method of claim 1, wherein the one or more anti-CGRP antagonist antibodies acts peripherally on administration.

20. The method of claim 1, wherein at least one of the one or more anti-CGRP antagonist antibodies: (a) binds to CGRP; (b) blocks CGRP from binding to its receptor; (c) blocks or decreases CGRP receptor activation; (d) inhibits blocks, suppresses or reduces CGRP biological activity; (e) increases clearance of CGRP; and/or (g) inhibits CGRP synthesis, production or release.

21. The method of claim 1, wherein administering the one or more anti-CGRP antagonist antibodies reduces serum glucose by at least 10%.

22. The method of claim 1, wherein the subject has a blood glucose level above 126 mg/dl.

23. The method of claim 1, wherein the subject does not have peripheral vascular disease and/or peripheral neuropathy.


SEQUENCE LISTING (174 sequences)

SEQ ID NO: 1 - protein, 122 aa, Artificial Sequence
  Description of Artificial Sequence Synthetic polypeptide
EVQLVESGGGLVQPGGSLRLSCAASGFTFSNYWISWVRQAPGKGLEWVAEIRSESDASATHYAEAVKGRFTISRDNAKNSLYLQMNSLRAEDTAVYYCLAYFDYGLAIQNYWGQGTLVTVSS

SEQ ID NO: 2 - protein, 107 aa, Artificial Sequence
  Description of Artificial Sequence Synthetic polypeptide
EIVLTQSPATLSLSPGERATLSCKASKRVTTYVSWYQQKPGQAPRLLIYGASNRYLGIPARFSGSGSGTDFTLTISSLEPEDFAVYYCSQSYNYPYTFGQGTKLEIK

SEQ ID NO: 3 - protein, 10 aa, Artificial Sequence
  Description of Artificial Sequence Synthetic peptide
GFTFSNYWIS

SEQ ID NO: 4 - protein, 19 aa, Artificial Sequence
  Description of Artificial Sequence Synthetic peptide
EIRSESDASATHYAEAVKG

SEQ ID NO: 5 - protein, 11 aa, Artificial Sequence
  Description of Artificial Sequence Synthetic peptide
YFDYGLAIQNY

SEQ ID NO: 6 - protein, 11 aa, Artificial Sequence
  Description of Artificial Sequence Synthetic peptide
KASKRVTTYVS

SEQ ID NO: 7 - protein, 7 aa, Artificial Sequence
  Description of Artificial Sequence Synthetic peptide
GASNRYL

SEQ ID NO: 8 - protein, 9 aa, Artificial Sequence
  Description of Artificial Sequence Synthetic peptide
SQSYNYPYT

SEQ ID NO: 9 - DNA, 366 bp, Artificial Sequence
  Description of Artificial Sequence Synthetic polynucleotide
gaagttcagctggttgaatccggtggtggtctggttcagccaggtggttccctgcgtctgtcctgcgctgcttccggtttcaccttctccaactactggatctcctgggttcgtcaggctcctggtaaaggtctggaatgggttgctgaaatccgttccgaatccgacgcgtccgctacccattacgctgaagctgttaaaggtcgtttcaccatctcccgtgacaacgctaagaactccctgtacctgcagatgaactccctgcgtgctgaagacaccgctgtttactactgcctggcttactttgactacggtctggctatccagaactactggggtcagggtaccctggttaccgtttcctcc

SEQ ID NO: 10 - DNA, 321 bp, Artificial Sequence
  Description of Artificial Sequence Synthetic polynucleotide
gaaatcgttctgacccagtccccggctaccctgtccctgtccccaggtgaacgtgctaccctgtcctgcaaagcttccaaacgggttaccacctacgtttcctggtaccagcagaaacccggtcaggctcctcgtctgctgatctacggtgcttccaaccgttacctcggtatcccagctcgtttctccggttccggttccggtaccgacttcaccctgaccatctcctccctggaacccgaagacttcgctgtttactactgcagtcagtcctacaactacccctacaccttcggtcagggtaccaaactggaaatcaaa

SEQ ID NO: 11 - protein, 448 aa, Artificial Sequence
  Description of Artificial Sequence Synthetic polypeptide
EVQLVESGGGLVQPGGSLRLSCAASGFTFSNYWISWVRQAPGKGLEWVAEIRSESDASATHYAEAVKGRFTISRDNAKNSLYLQMNSLRAEDTAVYYCLAYFDYGLAIQNYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Artificial Sequence
  Description of Artificial Sequence Synthetic polypeptide
EIVLTQSPATLSLSPGERATLSCKASKRVTTYVSWYQQKPGQAPRLLIYGASNRYLGIPARFSGSGSGTDFTLTISSLEPEDFAVYYCSQSYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1347 bp, Artificial Sequence
  Description of Artificial Sequence Synthetic polynucleotide
gaagttcagctggttgaatccggtggtggtctggttcagccaggtggttccctgcgtctgtcctgcgctgcttccggtttcaccttctccaactactggatctcctgggttcgtcaggctcctggtaaaggtctggaatgggttgctgaaatccgttccgaatccgacgcgtccgctacccattacgctgaagctgttaaaggtcgtttcaccatctcccgtgacaacgctaagaactccctgtacctgcagatgaactccctgcgtgctgaagacaccgctgtttactactgcctggcttactttgactacggtctggctatccagaactactggggtcagggtaccctggttaccgtttcctccgcctccaccaagggcccatctgtcttcccactggccccatgctcccgcagcacctccgagagcacagccgccctgggctgcctggtcaaggactacttcccagaacctgtgaccgtgtcctggaactctggcgctctgaccagcggcgtgcacaccttcccagctgtcctgcagtcctcaggtctctactccctcagcagcgtggtgaccgtgccatccagcaacttcggcacccagacctacacctgcaacgtagatcacaagccaagcaacaccaaggtcgacaagaccgtggagagaaagtgttgtgtggagtgtccaccttgtccagcccctccagtggccggaccatccgtgttcctgttccctccaaagccaaaggacaccctgatgatctccagaaccccagaggtgacctgtgtggtggtggacgtgtcccacgaggacccagaggtgcagttcaactggtatgtggacggagtggaggtgcacaacgccaagaccaagccaagagaggagcagttcaactccaccttcagagtggtgagcgtgctgaccgtggtgcaccaggactggctgaacggaaaggagtataagtgtaaggtgtccaacaagggactgccatccagcatcgagaagaccatctccaagaccaagggacagccaagagagccacaggtgtataccctgcccccatccagagaggagatgaccaagaaccaggtgtccctgacctgtctggtgaagggattctatccatccgacatcgccgtggagtgggagtccaacggacagccagagaacaactataagaccacccctccaatgctggactccgacggatccttcttcctgtattccaagctgaccgtggacaagtccagatggcagcagggaaacgtgttctcttgttccgtgatgcacgaggccctgcacaaccactatacccagaagagcctgtccctgtctccaggaaagtaa

SEQ ID NO: 14 - DNA, 645 bp, Artificial Sequence
  Description of Artificial Sequence Synthetic polynucleotide
gaaatcgttctgacccagtccccggctaccctgtccctgtccccaggtgaacgtgctaccctgtcctgcaaagcttccaaacgggttaccacctacgtttcctggtaccagcagaaacccggtcaggctcctcgtctgctgatctacggtgcttccaaccgttacctcggtatcccagctcgtttctccggttccggttccggtaccgacttcaccctgaccatctcctccctggaacccgaagacttcgctgtttactactgcagtcagtcctacaactacccctacaccttcggtcagggtaccaaactggaaatcaaacgcactgtggctgcaccatctgtcttcatcttccctccatctgatgagcagttgaaatccggaactgcctctgttgtgtgcctgctgaataacttctatccgcgcgaggccaaagtacagtggaaggtggataacgccctccaatccggtaactcccaggagagtgtcacagagcaggacagcaaggacagcacctacagcctcagcagcaccctgaccctgagcaaagcagactacgagaaacacaaagtctacgcctgcgaagtcacccatcagggcctgagttctccagtcacaaagagcttcaaccgcggtgagtgctaa

SEQ ID NO: 15 - protein, 37 aa, Homo sapiens
  C-term amidated
ACDTATCVTHRLAGLLSRSGGVVKNNFVPTNVGSKAF

SEQ ID NO: 16 - protein, 30 aa, Homo sapiens
  C-term amidated
VTHRLAGLLSRSGGVVKNNFVPTNVGSKAF

SEQ ID NO: 17 - protein, 19 aa, Homo sapiens
  C-term amidated
SGGVVKNNFVPTNVGSKAF

SEQ ID NO: 18 - protein, 19 aa, Homo sapiens
  C-term amidated
SGGVVKNNFVATNVGSKAF

SEQ ID NO: 19 - protein, 19 aa, Homo sapiens
  C-term amidated
SGGVVKNNFVPTNVGSAAF

SEQ ID NO: 20 - protein, 19 aa, Homo sapiens
  C-term amidated
SGGVVKNNFVPTNVGSEAF

SEQ ID NO: 21 - protein, 19 aa, Homo sapiens
  C-term amidated
SGGVVKNNFVPTNVGSMAF

SEQ ID NO: 22 - protein, 19 aa, Homo sapiens
  C-term amidated
SGGVVKNNFVPTNVGSQAF

SEQ ID NO: 23 - protein, 19 aa, Homo sapiens
  C-term amidated
SGGVVKNNFVPTNVGSKAA

SEQ ID NO: 24 - protein, 14 aa, Homo sapiens
  C-term amidated
NNFVPTNVGSKAFA

SEQ ID NO: 25 - protein, 13 aa, Homo sapiens
  C-term amidated
NNFVPTNVGSKAF

SEQ ID NO: 26 - protein, 13 aa, Homo sapiens
  C-term amidated
NNAVPTNVGSKAF

SEQ ID NO: 27 - protein, 13 aa, Homo sapiens
  C-term amidated
NNFAPTNVGSKAF

SEQ ID NO: 28 - protein, 13 aa, Homo sapiens
  C-term amidated
NNFVATNVGSKAF

SEQ ID NO: 29 - protein, 13 aa, Homo sapiens
  C-term amidated
NNFVPANVGSKAF

SEQ ID NO: 30 - protein, 13 aa, Homo sapiens
  C-term amidated
NNFVPTAVGSKAF

SEQ ID NO: 31 - protein, 13 aa, Homo sapiens
  C-term amidated
NNFVPTNAGSKAF

SEQ ID NO: 32 - protein, 13 aa, Homo sapiens
  C-term amidated
NNFVPTNVASKAF

SEQ ID NO: 33 - protein, 13 aa, Homo sapiens
  C-term amidated
NNFVPTNVGAKAF

SEQ ID NO: 34 - protein, 13 aa, Homo sapiens
  C-term amidated
NNFVPTNVGSKAA

SEQ ID NO: 35 - protein, 12 aa, Homo sapiens
  C-term amidated
NFVPTNVGSKAF

SEQ ID NO: 36 - protein, 19 aa, Homo sapiens
SGGVVKNNFVPTNVGSKAF

SEQ ID NO: 37 - protein, 18 aa, Homo sapiens
SGGVVKNNFVPTNVGSKA

SEQ ID NO: 38 - protein, 36 aa, Homo sapiens
ACDTATCVTHRLAGLLSRSGGVVKNNFVPTNVGSKA

SEQ ID NO: 39 - protein, 19 aa, Homo sapiens
ACDTATCVTHRLAGLLSRS

SEQ ID NO: 40 - protein, 13 aa, Homo sapiens
ACDTATCVTHRLA

SEQ ID NO: 41 - protein, 37 aa, Rattus sp.
  C-term amidated
SCNTATCVTHRLAGLLSRSGGVVKDNFVPTNVGSEAF

SEQ ID NO: 42 - protein, 19 aa, Rattus sp.
  C-term amidated
SGGVVKDNFVPTNVGSEAF

SEQ ID NO: 43 - protein, 37 aa, Homo sapiens
  C-term amidated
ACNTATCVTHRLAGLLSRSGGMVKSNFVPTNVGSKAF

SEQ ID NO: 44 - protein, 37 aa, Rattus sp.
  C-term amidated
SCNTATCVTHRLAGLLSRSGGVVKDNFVPTNVGSKAF

SEQ ID NO: 45 - protein, 32 aa, Homo sapiens
  C-term amidated
CGNLSTCMLGTYTQDFNKFHTFPQTAIGVGAP

SEQ ID NO: 46 - protein, 37 aa, Homo sapiens [sequence omitted]

SEQ ID NO: 47 - protein, 52 aa, Homo sapiens
  C-term amidated
YRQSMNNFQGLRSFGCRFGTCTVQKLAHQIYQFTDKDKDNVAPRSKISPQGY

SEQ ID NO: 48 - protein, 5 aa, Unknown
  Description of Unknown CGRP C-terminal peptide
GSKAF

SEQ ID NO: 49 - DNA, 20 bp, Artificial Sequence
  Description of Artificial Sequence Synthetic primer
ctaaggccaaccgtgaaaag

SEQ ID NO: 50 - DNA, 20 bp, Artificial Sequence
  Description of Artificial Sequence Synthetic primer
accagaggcatacagggaca

SEQ ID NO: 51 - DNA, 19 bp, Artificial Sequence
  Description of Artificial Sequence Synthetic primer
ggccctctctagttcccag

SEQ ID NO: 52 - DNA, 21 bp, Artificial Sequence
  Description of Artificial Sequence Synthetic primer
tagccatcaaacctgttgagc

SEQ ID NO: 53 - DNA, 20 bp, Artificial Sequence
  Description of Artificial Sequence Synthetic primer
agtggaatggcgagaagatg

SEQ ID NO: 54 - DNA, 20 bp, Artificial Sequence
  Description of Artificial Sequence Synthetic primer
acaagggctctgtcagaagg

SEQ ID NO: 55 - DNA, 23 bp, Artificial Sequence
  Description of Artificial Sequence Synthetic primer
acaccgagatttccttcaaactg

SEQ ID NO: 56 - protein, 6 aa, Artificial Sequence
  Description of Artificial Sequence Synthetic 6xHis tag
HHHHHH

SEQ ID NO: 57 - protein, 15 aa, Artificial Sequence
  Description of Artificial Sequence Synthetic peptide
GGGGSGGGGSGGGGS

SEQ ID NO: 58 - DNA, 25 bp, Artificial Sequence
  Description of Artificial Sequence Synthetic primer
ccatctagggttatgatgctcttca

SEQ ID NO: 59 - DNA, 21 bp, Artificial Sequence
  Description of Artificial Sequence Synthetic primer
ggtaagtgccgagctgagatg

SEQ ID NO: 60 - DNA, 19 bp, Artificial Sequence
  Description of Artificial Sequence Synthetic primer
cgacctgtgcaaccctttc

SEQ ID NO: 61 - DNA, 22 bp, Artificial Sequence
  Description of Artificial Sequence Synthetic primer
tcatacttcggttgcatgaagg

SEQ ID NO: 62 - DNA, 19 bp, Artificial Sequence
  Description of Artificial Sequence Synthetic primer
agacctctcgaacctgccc

SEQ ID NO: 63 - DNA, 21 bp, Artificial Sequence
  Description of Artificial Sequence Synthetic primer
cagtgcctttgaggtcaatct

SEQ ID NO: 64 - DNA, 21 bp, Artificial Sequence
  Description of Artificial Sequence Synthetic primer
ccagcaggcgaacttcttctt

SEQ ID NO: 65 - DNA, 23 bp, Artificial Sequence
  Description of Artificial Sequence Synthetic primer
ttaaaaacctggatcggaaccaa

SEQ ID NO: 66 - DNA, 23 bp, Artificial Sequence
  Description of Artificial Sequence Synthetic primer
gcattagcttcagatttacgggt

SEQ ID NO: 67 - DNA, 23 bp, Artificial Sequence
  Description of Artificial Sequence Synthetic primer
ttctctgtaccatgacactctgc

SEQ ID NO: 68 - DNA, 21 bp, Artificial Sequence
  Description of Artificial Sequence Synthetic primer
cgtggaatcttccggctgtag

SEQ ID NO: 69 - DNA, 20 bp, Artificial Sequence
  Description of Artificial Sequence Synthetic primer
gctgctttgcctacctctcc

SEQ ID NO: 70 - DNA, 21 bp, Artificial Sequence
  Description of Artificial Sequence Synthetic primer
tcgagtgacaaacacgactgc

SEQ ID NO: 71 - DNA, 21 bp, Artificial Sequence
  Description of Artificial Sequence Synthetic primer
gctgctttcagcatccaagtg

SEQ ID NO: 72 - DNA, 19 bp, Artificial Sequence
  Description of Artificial Sequence Synthetic primer
ccagggacaccgactactg

SEQ ID NO: 73 - DNA, 21 bp, Artificial Sequence
  Description of Artificial Sequence Synthetic primer
tctacgcagtgcttctttgcc

SEQ ID NO: 74 - DNA, 23 bp, Artificial Sequence
  Description of Artificial Sequence Synthetic primer
aagggggatcttcagctttagta

SEQ ID NO: 75 - DNA, 24 bp, Artificial Sequence
  Description of Artificial Sequence Synthetic primer
ttctccagctgttcaccttgacct

SEQ ID NO: 76 - DNA, 24 bp, Artificial Sequence
  Description of Artificial Sequence Synthetic primer
gttgcaagagaaacatggcccgaa

SEQ ID NO: 77 - DNA, 22 bp, Artificial Sequence
  Description of Artificial Sequence Synthetic primer
cagcctttcctactaccattcc

SEQ ID NO: 78 - DNA, 20 bp, Artificial Sequence
  Description of Artificial Sequence Synthetic primer
acagatctgcgcaaaagtcc

SEQ ID NO: 79 - DNA, 21 bp, Artificial Sequence
  Description of Artificial Sequence Synthetic primer
tgctcttctgtatcgcccagt

SEQ ID NO: 80 - DNA, 21 bp, Artificial Sequence
  Description of Artificial Sequence Synthetic primer
gccgtgttaaggaatctgctg

SEQ ID NO: 81 - DNA, 21 bp, Artificial Sequence
  Description of Artificial Sequence Synthetic primer
agcctggcaactactctgaca

SEQ ID NO: 82 - DNA, 21 bp, Artificial Sequence
  Description of Artificial Sequence Synthetic primer
gaagcacgttcttgttaggca

SEQ ID NO: 83 - DNA, 23 bp, Artificial Sequence
  Description of Artificial Sequence Synthetic primer
ctgggattcacctcaagaacatc

SEQ ID NO: 84 - DNA, 19 bp, Artificial Sequence
  Description of Artificial Sequence Synthetic primer
cagggtcaaggcaagcctc

SEQ ID NO: 85 - DNA, 21 bp, Artificial Sequence
  Description of Artificial Sequence Synthetic primer
tgcgttgtgtttgcttaaccg

SEQ ID NO: 86 - DNA, 21 bp, Artificial Sequence
  Description of Artificial Sequence Synthetic primer
agctatgacttccaccgtagg

SEQ ID NO: 87 - DNA, 21 bp, Artificial Sequence
  Description of Artificial Sequence Synthetic primer
ccaagtgctgccgtcattttc

SEQ ID NO: 88 - DNA, 21 bp, Artificial Sequence
  Description of Artificial Sequence Synthetic primer
ggctcgcagggatgatttcaa

SEQ ID NO: 89 - DNA, 21 bp, Artificial Sequence
  Description of Artificial Sequence Synthetic primer
tgcatcagtgacggtaaacca

SEQ ID NO: 90 - DNA, 21 bp, Artificial Sequence
  Description of Artificial Sequence Synthetic primer
ttcttcagccgtgcaacaatc

SEQ ID NO: 91 - DNA, 24 bp, Artificial Sequence
  Description of Artificial Sequence Synthetic primer
cagtgtggttgcacgtctccaatc

SEQ ID NO: 92 - DNA, 21 bp, Artificial Sequence
  Description of Artificial Sequence Synthetic primer
tgaaccaaagttgaccaccag

SEQ ID NO: 93 - DNA, 20 bp, Artificial Sequence
  Description of Artificial Sequence Synthetic primer
tcggctcctttcctcactca

SEQ ID NO: 94 - DNA, 21 bp, Artificial Sequence
  Description of Artificial Sequence Synthetic primer
ctcatagggttgttcgctcgg

SEQ ID NO: 95 - DNA, 21 bp, Artificial Sequence
  Description of Artificial Sequence Synthetic primer
aatcgcctatggttgttgacc

SEQ ID NO: 96 - DNA, 21 bp, Artificial Sequence
  Description of Artificial Sequence Synthetic primer
ttggtatggtttcacgactgg

SEQ ID NO: 97 - DNA, 21 bp, Artificial Sequence
  Description of Artificial Sequence Synthetic primer
ccccagtgtccttacagagtg

SEQ ID NO: 98 - DNA, 19 bp, Artificial Sequence
  Description of Artificial Sequence Synthetic primer
gtgcccagagtggatgtct

SEQ ID NO: 99 - DNA, 20 bp, Artificial Sequence
  Description of Artificial Sequence Synthetic primer
tcacagggcacattcaccag

SEQ ID NO: 100 - DNA, 22 bp, Artificial Sequence
  Description of Artificial Sequence Synthetic primer
catcatgacgtttggcaatgtt

SEQ ID NO: 101 - DNA, 19 bp, Artificial Sequence
  Description of Artificial Sequence Synthetic primer
tcctcctcagaccgctttt

SEQ ID NO: 102 - DNA, 21 bp, Artificial Sequence
  Description of Artificial Sequence Synthetic primer
cctggttcatcatcgctaatc

SEQ ID NO: 103 - DNA, 21 bp, Artificial Sequence
  Description of Artificial Sequence Synthetic primer
aggctctcacttcttggctga

SEQ ID NO: 104 - DNA, 22 bp, Artificial Sequence
  Description of Artificial Sequence Synthetic primer
gggacactccttacttggttgt

SEQ ID NO: 105 - DNA, 21 bp, Artificial Sequence
  Description of Artificial Sequence Synthetic primer
tggatctgggagcatcaaggt

SEQ ID NO: 106 - DNA, 21 bp, Artificial Sequence
  Description of Artificial Sequence Synthetic primer
tggaagtgcggatgtagtcag

SEQ ID NO: 107 - DNA, 23 bp, Artificial Sequence
  Description of Artificial Sequence Synthetic primer
tagtccttcctaccccaatttcc

SEQ ID NO: 108 - DNA, 21 bp, Artificial Sequence
  Description of Artificial Sequence Synthetic primer
ttggtccttagccactccttc

SEQ ID NO: 109 - DNA, 21 bp, Artificial Sequence
  Description of Artificial Sequence Synthetic primer
tcagccacctgtgacgaattt

SEQ ID NO: 110 - DNA, 21 bp, Artificial Sequence
  Description of Artificial Sequence Synthetic primer
tgagagtgcaatttggactgg

SEQ ID NO: 111 - DNA, 21 bp, Artificial Sequence
  Description of Artificial Sequence Synthetic primer
gctctctacctggtgtgtggg

SEQ ID NO: 112 - DNA, 21 bp, Artificial Sequence
  Description of Artificial Sequence Synthetic primer
caaggtctgaaggtcacctgc

SEQ ID NO: 113 - DNA, 19 bp, Artificial Sequence
  Description of Artificial Sequence Synthetic primer
gtccaggcactggagcttt

SEQ ID NO: 114 - DNA, 19 bp, Artificial Sequence
  Description of Artificial Sequence Synthetic primer
gcgcttcactctttcacga

SEQ ID NO: 115 - DNA, 19 bp, Artificial Sequence
  Description of Artificial Sequence Synthetic primer
gatcaccgacaagggcctg

SEQ ID NO: 116 - DNA, 21 bp, Artificial Sequence
  Description of Artificial Sequence Synthetic primer
ggctatgaaactcgtactgcc

SEQ ID NO: 117 - DNA, 21 bp, Artificial Sequence
  Description of Artificial Sequence Synthetic primer
cccaacgtcatttctgtccgt

SEQ ID NO: 118 - DNA, 20 bp, Artificial Sequence
  Description of Artificial Sequence Synthetic primer
tctaccaggggccgatcatt

SEQ ID NO: 119 - DNA, 18 bp, Artificial Sequence
  Description of Artificial Sequence Synthetic primer
tcagagcccacgtcgatt

SEQ ID NO: 120 - DNA, 20 bp, Artificial Sequence
  Description of Artificial Sequence Synthetic primer
tagtcagggtttgcctggaa

SEQ ID NO: 121 - DNA, 20 bp, Artificial Sequence
  Description of Artificial Sequence Synthetic primer
tgcgtgcaagcccagtatag

SEQ ID NO: 122 - DNA, 22 bp, Artificial Sequence
  Description of Artificial Sequence Synthetic primer
tggtgtattccgagccataagt

SEQ ID NO: 123 - DNA, 19 bp, Artificial Sequence
  Description of Artificial Sequence Synthetic primer
ccctgccattgttaagacc

SEQ ID NO: 124 - DNA, 19 bp, Artificial Sequence
  Description of Artificial Sequence Synthetic primer
tgctgctgttcctgttttc

SEQ ID NO: 125 - DNA, 21 bp, Artificial Sequence
  Description of Artificial Sequence Synthetic primer
cacaagagctgacccaatggt

SEQ ID NO: 126 - DNA, 23 bp, Artificial Sequence
  Description of Artificial Sequence Synthetic primer
gatcgcactntggtattctngga

SEQ ID NO: 127 - DNA, 19 bp, Artificial Sequence
  Description of Artificial Sequence Synthetic primer
cagcacggtgaagccattc

SEQ ID NO: 128 - DNA, 17 bp, Artificial Sequence
  Description of Artificial Sequence Synthetic primer
gcgtgcatccgcttgtg

SEQ ID NO: 129 - DNA, 22 bp, Artificial Sequence
  Description of Artificial Sequence Synthetic primer
agaaaatgaagcggacactctt

SEQ ID NO: 130 - DNA, 18 bp, Artificial Sequence
  Description of Artificial Sequence Synthetic primer
ttgccagttttgggcttc

SEQ ID NO: 131 - DNA, 23 bp, Artificial Sequence
  Description of Artificial Sequence Synthetic primer
tgccatcattctttgcatcttga

SEQ ID NO: 132 - DNA, 21 bp, Artificial Sequence
  Description of Artificial Sequence Synthetic primer
ccgtgaacttctgaacagcct

SEQ ID NO: 133 - DNA, 21 bp, Artificial Sequence
  Description of Artificial Sequence Synthetic primer
atggtcacccacagcaagttt

SEQ ID NO: 134 - DNA, 21 bp, Artificial Sequence
  Description of Artificial Sequence Synthetic primer
tccagtagaatccgctctcct

SEQ ID NO: 135 - DNA, 21 bp, Artificial Sequence
  Description of Artificial Sequence Synthetic primer
gagggaggaagccgtaatgag

SEQ ID NO: 136 - DNA, 19 bp, Artificial Sequence
  Description of Artificial Sequence Synthetic primer
cggcacagttttggttccg

SEQ ID NO: 137 - DNA, 20 bp, Artificial Sequence
  Description of Artificial Sequence Synthetic primer
agcctcagcagttctttgga

SEQ ID NO: 138 - DNA, 21 bp, Artificial Sequence
  Description of Artificial Sequence Synthetic primer
tctggccatgtccataaagag

SEQ ID NO: 139 - DNA, 21 bp, Artificial Sequence
  Description of Artificial Sequence Synthetic primer
gcaactcggacctggtcataa

SEQ ID NO: 140 - DNA, 19 bp, Artificial Sequence
  Description of Artificial Sequence Synthetic primer
cggcccgtgatgagaaact

SEQ ID NO: 141 - DNA, 20 bp, Artificial Sequence
  Description of Artificial Sequence Synthetic primer
gcaaacgctgttctgctcag

SEQ ID NO: 142 - DNA, 22 bp, Artificial Sequence
  Description of Artificial Sequence Synthetic primer
aggcgtctccctctattgtatt

SEQ ID NO: 143 - DNA, 23 bp, Artificial Sequence
  Description of Artificial Sequence Synthetic primer
ccctcacactcagatcatcttct

SEQ ID NO: 144 - DNA, 19 bp, Artificial Sequence
  Description of Artificial Sequence Synthetic primer
gctacgacgtgggctacag

SEQ ID NO: 145 - DNA, 17 bp, Artificial Sequence
  Description of Artificial Sequence Synthetic primer
gctgtgcccctcgacct

SEQ ID NO: 146 - DNA, 17 bp, Artificial Sequence
  Description of Artificial Sequence Synthetic primer
tgggcatcagcatcgct

SEQ ID NO: 147 - DNA, 21 bp, Artificial Sequence
  Description of Artificial Sequence Synthetic primer
actgccacacctccagtcatt

SEQ ID NO: 148 - DNA, 21 bp, Artificial Sequence
  Description of Artificial Sequence Synthetic primer
ctttgcctcactcaggattgg

SEQ ID NO: 149 - DNA, 21 bp, Artificial Sequence
  Description of Artificial Sequence Synthetic primer
caggtcactgtgcccttacca

SEQ ID NO: 150 - DNA, 21 bp, Artificial Sequence
  Description of Artificial Sequence Synthetic primer
cactacgttccaggatcccaa

SEQ ID NO: 151 - DNA, 20 bp, Artificial Sequence
  Description of Artificial Sequence Synthetic primer
gtgcctgacagtgcagaatc

SEQ ID NO: 152 - DNA, 20 bp, Artificial Sequence
  Description of Artificial Sequence Synthetic primer
cactgcccaggtacaggaag

SEQ ID NO: 153 - DNA, 21 bp, Artificial Sequence
  Description of Artificial Sequence Synthetic primer
agtnggggattcggntgntct

SEQ ID NO: 154 - DNA, 20 bp, Artificial Sequence
  Description of Artificial Sequence Synthetic primer
cccctcattccttaccaccc

SEQ ID NO: 155 - protein, 115 aa, Artificial Sequence
  Description of Artificial Sequence Synthetic polypeptide
EVQLQQSGPELVKPGASVKMSCKASGYTFTSSVMHWVKQKPGQGLEWIGYINPYNDGTKYNEKFKGKATLTSDKSSSTAYMELSSLTSEDSAVYYCAKGGNDGYWGQGTTLTVSS

SEQ ID NO: 156 - protein, 108 aa, Artificial Sequence
  Description of Artificial Sequence Synthetic polypeptide
EIVLTQSPTTMAASPGEKITITCSASSSISSIYLHWYQQKPGFSPKVLIYRASNLASGVPARFSGSGSGTSYSLTIGTMEAEDVATYYCQQGSTIPFTFGSGTKLEIK

SEQ ID NO: 157 - protein, 5 aa, Artificial Sequence
  Description of Artificial Sequence Synthetic peptide
SSVMH

SEQ ID NO: 158 - protein, 17 aa, Artificial Sequence
  Description of Artificial Sequence Synthetic peptide
YINPYNDGTKYNEKFKG

SEQ ID NO: 159 - protein, 6 aa, Artificial Sequence
  Description of Artificial Sequence Synthetic peptide
GGNDGY

SEQ ID NO: 160 - protein, 12 aa, Artificial Sequence
  Description of Artificial Sequence Synthetic peptide
SASSSISSIYLH

SEQ ID NO: 161 - protein, 7 aa, Artificial Sequence
  Description of Artificial Sequence Synthetic peptide
RASNLAS

SEQ ID NO: 162 - protein, 9 aa, Artificial Sequence
  Description of Artificial Sequence Synthetic peptide
QQGSTIPFT

SEQ ID NO: 163 - DNA, 345 bp, Artificial Sequence
  Description of Artificial Sequence Synthetic polynucleotide
gaggtccagctgcagcagtctggacctgagctggtaaagcctggggcttcagtgaagatgtcctgcaaggcttctggatacacattcactagctctgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggaactcagcagcctgacctctgaggactctgcggtctattactgtgcaaaagggggtaacgatggctactggggccaaggcactactctcacagtctcctca

SEQ ID NO: 164 - DNA, 324 bp, Artificial Sequence
  Description of Artificial Sequence Synthetic polynucleotide
gaaattgtgctcacccagtctccaaccaccatggctgcatctcccggggagaagatcactatcacctgtagtgccagctcaagtataagttccatttacttgcattggtatcagcagaagccaggattctcccctaaagtcttgatttatagggcatccaatctggcttctggagtcccagctcgcttcagtggcagtgggtctgggacctcttactctctcacaattggcaccatggaggctgaagatgttgccacttactactgccagcagggtagtactataccattcacgttcggctcggggacaaagttggaaataaaa

SEQ ID NO: 165 - protein, 216 aa, Artificial Sequence
  Description of Artificial Sequence Synthetic polypeptide
EVQLQQSGPELVKPGASVKMSCKASGYTFTSSVMHWVKQKPGQGLEWIGYINPYNDGTKYNEKFKGKATLTSDKSSSTAYMELSSLTSEDSAVYYCAKGGNDGYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRD

SEQ ID NO: 166 - protein, 215 aa, Artificial Sequence
  Description of Artificial Sequence Synthetic polypeptide
EIVLTQSPTTMAASPGEKITITCSASSSISSIYLHWYQQKPGFSPKVLIYRASNLASGVPARFSGSGSGTSYSLTIGTMEAEDVATYYCQQGSTIPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 167 - DNA, 648 bp, Artificial Sequence
  Description of Artificial Sequence Synthetic polynucleotide
gaggtccagctgcagcagtctggacctgagctggtaaagcctggggcttcagtgaagatgtcctgcaaggcttctggatacacattcactagctctgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggaactcagcagcctgacctctgaggactctgcggtctattactgtgcaaaagggggtaacgatggctactggggccaaggcactactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggat

SEQ ID NO: 168 - DNA, 648 bp, Artificial Sequence
  Description of Artificial Sequence Synthetic polynucleotide
gaaattgtgctcacccagtctccaaccaccatggctgcatctcccggggagaagatcactatcacctgtagtgccagctcaagtataagttccatttacttgcattggtatcagcagaagccaggattctcccctaaagtcttgatttatagggcatccaatctggcttctggagtcccagctcgcttcagtggcagtgggtctgggacctcttactctctcacaattggcaccatggaggctgaagatgttgccacttactactgccagcagggtagtactataccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttaa

SEQ ID NO: 169 - protein, 7 aa, Artificial Sequence
  Description of Artificial Sequence Synthetic peptide
GFTFSNY

SEQ ID NO: 170 - protein, 5 aa, Artificial Sequence
  Description of Artificial Sequence Synthetic peptide
NYWIS

SEQ ID NO: 171 - protein, 8 aa, Artificial Sequence
  Description of Artificial Sequence Synthetic peptide
RSESDASA

SEQ ID NO: 172 - protein, 10 aa, Artificial Sequence
  Description of Artificial Sequence Synthetic peptide
GYTFTSSVMH

SEQ ID NO: 173 - protein, 7 aa, Artificial Sequence
  Description of Artificial Sequence Synthetic peptide
GYTFTSS

SEQ ID NO: 174 - protein, 6 aa, Artificial Sequence
  Description of Artificial Sequence Synthetic peptide
NPYNDG